(19) United States
(12) Patent Application Publication
(10) Pub. No.: US 2020/0270335 A1
(43) Pub. Date: Aug. 27, 2020

(54) DKK2 CYSTEINE RICH DOMAIN 2 CONTAINING PROTEINS AND USES THEREOF

(71) Applicant: Biogen MA Inc., Cambridge, MA (US)

(72) Inventors: R. Blake Pepinsky, Arlington, MA (US); Richelle Sopko, Brookline, MA (US); Joshua Mugford, Waltham, MA (US); Andreas Lehmann, Belmont, MA (US); Joseph W. Arndt, Swampscott, MA (US); Nels E. Pederson, Mansfield, MA (US); Brenda K. Minesinger, Tewksbury, MA (US)

(21) Appl. No.: 16/063,423

(22) PCT Filed: Dec. 16, 2016
(86) PCT No.: PCT/US2016/067335

§ 371 Date: Jun. 18, 2018

Related U.S. Application Data
(60) Provisional application No. 62/387,116, filed on Dec. 23, 2015.

(51) Int. Cl. C07K 16/18; A61K 47/65; C07K 14/765

(52) U.S. Cl. CPC C07K 16/18; A61K 47/65; C07K 2319/74; C07K 2319/30; C07K 2319/31; C07K 14/765

(57) ABSTRACT

Proteins containing a DKK2 polypeptide or a fragment or variant thereof are described. These proteins contain human serum albumin sequences and/or include substitutions in the DKK2 polypeptide that decrease heparin binding. These proteins are useful in the treatment of disorders such as acute kidney injury and fibrosis.

### CROSS-REFERENCE TO RELATED APPLICATION

This application claims the benefit of priority to U.S. Provisional Appl. No. 62/387,116, filed Dec. 23, 2015, the contents of which are incorporated by reference in their entirety herein.

### BACKGROUND

Wnt signals are transduced by the Frizzled family of seven transmembrane domain receptors. Frizzled cell-surface receptors (Fzd) play an essential role in both the canonical and non-canonical Wnt signaling pathways. In the canonical pathway, upon activation of Fzd and low-density-lipoprotein receptor-related protein 5 and 6 (LRP5 and LRP6) by Wnt proteins, a signal is generated that prevents the phosphorylation and degradation of β-catenin. This allows β-catenin to translocate and accumulate in the nucleus and activate TCF/LEF target genes. The non-canonical Wnt signaling pathway is less well defined. There are at least two non-canonical Wnt signaling pathways that have been proposed, including the planar cell polarity (PCP) pathway and the Wnt/Ca⁺⁺ pathway.

Dickkopf 2 (DKK2) is a secreted polypeptide that can act as an antagonist of the canonical Wnt signaling pathway. DKK2 contains two cysteine rich domains, C1 and C2, each containing 10 conserved cysteines, separated by a variable-length spacer region. The C1 domain of human DKK2 protein is between amino acid positions 78 and 127 and the C2 domain of human DKK2 protein is between amino acid positions 183 and 256 of human DKK2. Wnt antagonism by DKK2 requires the binding of the C-terminal cysteine-rich domain of DKK2 (i.e., C2) to the Wnt coreceptor, LRP5/6. The DKK2-LRP5/6 complex antagonizes canonical Wnt signaling by inhibiting LRP5/6 interaction with Wnt and by forming a ternary complex with the transmembrane protein Kremen that promotes clathrin-mediated internalization of LRP5/6.

### SUMMARY

This application is based, at least in part, on the surprising discovery that the choice of fusion partner for a DKK2 polypeptide significantly affects the expression level, aggregation, disulfide scrambling, proteolytic lability, and activity of the DKK2 polypeptide. Specifically, human serum albumin (HSA) was identified as a highly effective fusion partner for DKK2 polypeptides. It was also discovered that deletion of the propeptide sequence of HSA can reduce heterogeneity of HSA-DKK2 fusion polypeptides. The invention is also based, at least in part, on the discovery that substitution of selected amino acid residues in DKK2 decreases heparin binding by variant DKK2 polypeptides. The HSA-DKK2-C2 fusion was found to exhibit improved pharmacokinetics relative to DKK2-C2, and the HSA-heparin binding DKK2-C2 mutants were found to exhibit improved pharmacokinetics relative to HSA-wildtype DKK2-C2.

In one aspect, the disclosure provides a polypeptide comprising a first amino acid sequence that comprises or consists of a sequence that is at least 90% identical to amino acids 21-605 of SEQ ID NO:24 that is directly linked or linked via a linker to a second amino acid sequence that comprises or consists of a sequence that is at least 90% identical to amino acids 3-88 of SEQ ID NO:2. The polypeptide binds to LRP5 and/or LRP6. In certain instances, the first amino acid sequence has improved affinity for FcRn relative to SEQ ID NO:50. The first amino acid sequence may be at the N- or C-terminus of the second amino acid sequence.

In certain embodiments of the first aspect, the first amino acid sequence is at least 95% identical to amino acids 21-605 of SEQ ID NO:24 and the second amino acid sequence is at least 95% identical to amino acids 3-88 of SEQ ID NO:2. In other embodiments, the first amino acid sequence is identical to amino acids 21-605 of SEQ ID NO:24 and the second amino acid sequence is at least 90% identical to amino acids 3-88 of SEQ ID NO:2. In yet other embodiments, the first amino acid sequence is identical to amino acids 21-605 of SEQ ID NO:24 and the second amino acid sequence is at least 95% identical to amino acids 3-88 of SEQ ID NO:2. In certain embodiments, the first amino acid sequence is identical to amino acids 21-605 of SEQ ID NO:24 and the second amino acid sequence is identical to amino acids 3-88 of SEQ ID NO:2. In some embodiments, the first amino acid sequence is directly linked to the second amino acid sequence. In some embodiments, the first amino acid sequence is linked to the second amino acid sequence via a linker. In certain embodiments, the linker is a peptide linker (e.g., glycine-serine, alanine-alanine-alanine).

In a second aspect, the disclosure provides a polypeptide comprising a first amino acid sequence that is at least 90%, at least 95%, at least 96%, at least 97%, at least 98%, at least 99%, or 100% identical to amino acids 21-612 of SEQ ID NO:14 that is directly linked or linked via a linker to a second amino acid sequence comprising a sequence that is at least 90%, at least 95%, at least 96%, at least 97%, at least 98%, at least 99%, or 100% identical to amino acids 620-703 of SEQ ID NO:14. The polypeptide binds to LRP5 and/or LRP6. In certain instances, the first amino acid sequence has improved affinity for FcRn relative to SEQ ID NO:50. In some embodiments, the first amino acid sequence is directly linked to the second amino acid sequence. In some embodiments, the first amino acid sequence is linked to the second amino acid sequence via a linker. In certain embodiments, the linker is a peptide linker (e.g., glycine-serine, alanine-alanine-alanine). In a particular embodiment, the polypeptide comprises a first amino acid sequence that is identical to amino acids 21-612 of SEQ ID NO:14 and a second amino acid sequence that is identical to amino acids 620-703 of SEQ ID NO:14.

In a third aspect, the disclosure provides a polypeptide comprising a first amino acid sequence that is at least 90%, at least 95%, at least 96%, at least 97%, at least 98%, at least 99%, or 100% identical to amino acids 21-612 of SEQ ID NO:14 that is directly linked or linked via a linker to a second amino acid sequence comprising a sequence that is at least 90%, at least 95%, at least 96%, at least 97%, at least 98%, at least 99%, or 100% identical to amino acids 616-703 of SEQ ID NO:14. The polypeptide binds to LRP5 and/or LRP6. In certain instances, the first amino acid sequence has improved affinity for FcRn relative to SEQ ID NO:50. In certain instances, the amino acid at position 617 of SEQ ID NO:14 is a proline instead of a serine. In some embodiments, the first amino acid sequence is linked to the second amino acid sequence via a linker. In certain embodiments, the linker is a peptide linker (e.g., glycine-serine, alanine-alanine-alanine). In a particular embodiment, the polypeptide comprises a first amino acid sequence that is identical to amino acids 21-612 of SEQ ID NO:14 and a second amino acid sequence that is identical to amino acids 616-703 of SEQ ID NO:14. In another embodiment, the polypeptide comprises a first amino acid sequence that is identical to amino acids 21-612 of SEQ ID NO:14 and a second amino acid sequence that is identical to amino acids 616-703 of SEQ ID NO:14 except that the amino acid at position 617 of SEQ ID NO:14 is a proline instead of a serine.

In a fourth aspect, the disclosure provides a polypeptide comprising a first amino acid sequence that is at least 90%, at least 95%, at least 96%, at least 97%, at least 98%, at least 99%, or 100% identical to amino acids 21-612 of SEQ ID NO:14 that is directly linked or linked via a linker to a second amino acid sequence comprising a sequence that is at least 90%, at least 95%, at least 96%, at least 97%, at least 98%, at least 99%, or 100% identical to amino acids 622-703 of SEQ ID NO:14. The polypeptide binds to LRP5 and/or LRP6. In certain instances, the first amino acid sequence has improved affinity for FcRn relative to SEQ ID NO:50. In some embodiments, the first amino acid sequence is linked to the second amino acid sequence via a linker. In certain embodiments, the linker is a peptide linker. In a particular embodiment, the polypeptide comprises a first amino acid sequence that is identical to amino acids 21-612 of SEQ ID NO:14 and a second amino acid sequence that is identical to amino acids 622-703 of SEQ ID NO:14.

In a fifth aspect, the disclosure relates to a polypeptide comprising an amino acid sequence that is at least 90% identical to amino acids 3-88 of SEQ ID NO:2, wherein the amino acid sequence comprises at least one amino acid substitution, relative to SEQ ID NO:2. The polypeptide binds to LRP5 and/or LRP6. The amino acid substitution is selected from the group consisting of (a) an amino acid other than arginine at the position corresponding to position 14 of SEQ ID NO:2; (b) an amino acid other than arginine at the position corresponding to position 26 of SEQ ID NO:2; (c) an amino acid other than lysine at the position corresponding to position 31 of SEQ ID NO:2; (d) an amino acid other than lysine at the position corresponding to position 45 of SEQ ID NO:2; (e) an amino acid other than lysine at the position corresponding to position 49 of SEQ ID NO:2; (f) an amino acid other than histidine at the position corresponding to position 52 of SEQ ID NO:2; (g) an amino acid other than lysine at the position corresponding to position 69 of SEQ ID NO:2; (h) an amino acid other than lysine at the position corresponding to position 72 of SEQ ID NO:2; (i) an amino acid other than serine at the position corresponding to position 77 of SEQ ID NO:2; and (j) an amino acid other than lysine at the position corresponding to position 79 of SEQ ID NO:2.

In certain embodiments of the fifth aspect, the amino acid sequence is at least 95% identical to amino acids 3-88 of SEQ ID NO:2. In some embodiments, the polypeptide comprises two amino acid substitutions selected from the group consisting of (a) through (j). In other embodiments, the polypeptide comprises three amino acid substitutions selected from the group consisting of (a) through (j). In yet other embodiments, the polypeptide comprises four amino acid substitutions selected from the group consisting of (a) through (j). In certain embodiments, the polypeptide contains an amino acid other than lysine at the position corresponding to position 45 of SEQ ID NO:2. In specific embodiments, the amino acid at the position corresponding to position 45 of SEQ ID NO:2 is glutamic acid or serine. In certain embodiments, the polypeptide contains an amino acid other than lysine at the position corresponding to position 49 of SEQ ID NO:2. In specific embodiments, the amino acid at the position corresponding to position 49 of SEQ ID NO:2 is glutamic acid or asparagine. In certain embodiments, the polypeptide contains an amino acid other than lysine at the position corresponding to position 79 of SEQ ID NO:2. In specific embodiments, the amino acid at the position corresponding to position 79 of SEQ ID NO:2 is glutamic acid or serine. In certain embodiments, the polypeptide contains an amino acid other than histidine at the position corresponding to position 52 of SEQ ID NO:2. In specific embodiments, the amino acid at the position corresponding to position 52 of SEQ ID NO:2 is glutamic acid. In certain embodiments, the polypeptide contains an amino acid other than lysine at the position corresponding to position 45 of SEQ ID NO:2 and an amino acid other than lysine at the position corresponding to position 49 of SEQ ID NO:2. In specific embodiments, the amino acids at the positions corresponding to positions 45 and 49 of SEQ ID NO:2 are glutamic acid. In specific embodiments, the amino acids at the positions corresponding to positions 45 and 49 of SEQ ID NO:2 are serine. In certain embodiments, the polypeptide contains an amino acid other than lysine at the position corresponding to position 45 of SEQ ID NO:2 and an amino acid other than lysine at the position corresponding to position 79 of SEQ ID NO:2. In specific embodiments, the amino acids at the positions corresponding to positions 45 and 79 of SEQ ID NO:2 are glutamic acid. In certain embodiments, the polypeptide contains an amino acid other than lysine at the position corresponding to position 45 of SEQ ID NO:2 and an amino acid other than histidine at the position corresponding to position 52 of SEQ ID NO:2. In specific embodiments, the amino acids at the positions corresponding to positions 45 and 52 of SEQ ID NO:2 are glutamic acid. In specific embodiments, the amino acid at the position corresponding to position 45 of SEQ ID NO:2 is serine and the amino acid at the position corresponding to position 52 of SEQ ID NO:2 is threonine. In certain embodiments, the polypeptide contains an amino acid other than lysine at the position corresponding to position 69 of SEQ ID NO:2 and an amino acid other than lysine at the position corresponding to position 72 of SEQ ID NO:2. In specific embodiments, the amino acids at the positions corresponding to positions 69 and 72 of SEQ ID NO:2 are glutamic acid. In certain embodiments, the polypeptide contains an amino acid other than serine at the position corresponding to position 77 of SEQ ID NO:2 and an amino acid other than lysine at the position corresponding to position 79 of SEQ ID NO:2. In specific embodiments, the amino acid at the position corresponding to position 77 of SEQ ID NO:2 is asparagine and the amino acid at the position corresponding to position 79 of SEQ ID NO:2 is serine. In specific embodiments, the amino acid sequence of the polypeptide is identical to amino acids 608-693 of SEQ ID NO:32; amino acids 608-693 of SEQ ID NO:33; amino acids 608-693 of SEQ ID NO:36; amino acids 608-693 of SEQ ID NO:40; or amino acids 608-693 of SEQ ID NO:41. In some embodiments, the polypeptide is linked either directly or via a linker to the C-terminus of a second polypeptide comprising an amino acid sequence that is at least 90% identical to amino acids 21-605 of SEQ ID NO:24. In other embodiments, the polypeptide is linked either directly or via a linker to the C-terminus of a second polypeptide comprising amino acids 21-605 of SEQ ID NO:24. In specific embodiments, the amino acid sequence of the polypeptide is identical to amino acids 21-693 of SEQ ID NO:32; amino acids 21-693 of SEQ ID NO:33; amino acids 21-693 of SEQ ID NO:36; amino acids 21-693 of SEQ ID NO:40; or amino acids 21-693 of SEQ ID NO:41. In some embodiments, the polypeptide is linked either directly or via a linker to the N-terminus of a second polypeptide comprising an amino acid sequence that is at least 90% identical to amino acids 21-605 of SEQ ID NO:24. In other embodiments, the polypeptide is linked either directly or via a linker to the N-terminus of a second polypeptide comprising amino acids 21-605 of SEQ ID NO:24. In certain embodiments, the polypeptide is linked to the second polypeptide via a linker. The linker may be a peptide linker (e.g., glycine-serine, alanine-alanine-alanine).

In another aspect, the disclosure also provides pharmaceutical compositions comprising a DKK2 polypeptide (e.g., a HSA-DKK2-C2 heparin binding mutant) described herein. In yet another aspect, the disclosure provides a method for treating an acute kidney injury in a human subject in need thereof. The method involves administering to the human subject in need thereof a therapeutically effective amount of a DKK2 polypeptide (e.g., a HSA-DKK2-C2 heparin binding mutant) described herein.

In another aspect, the disclosure provides a method for treating fibrosis in a human subject in need thereof. The method involves administering to the human subject in need thereof a therapeutically effective amount of a DKK2 polypeptide (e.g., a HSA-DKK2-C2 heparin binding mutant) described herein.

In a further aspect, the disclosure provides a nucleic acid that encodes a DKK2 polypeptide (e.g., a HSA-DKK2-C2 heparin binding mutant) described herein.

In another aspect, the disclosure provides a vector comprising the nucleic acid described above.

In a further aspect, the disclosure encompasses host cells comprising the nucleic acid or vector described above.

In yet another aspect, the disclosure relates to a method of making a DKK2 polypeptide (e.g., a HSA-DKK2-C2 heparin binding mutant) described herein. The method involves culturing a host cell comprising a nucleic acid encoding the DKK2 polypeptide under conditions that lead to the expression of the polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

### BRIEF DESCRIPTION OF THE DRAWINGS

FIG. 1 is a photograph of a gel showing the analysis of conditioned medium of His-DKK2 expressing cells by SDS-PAGE/western analysis. DKK2 expression was assessed using an ant-DKK2 rabbit polyclonal antibody that recognizes the C2 domain of DKK2. Molecular weights in kDa of gel standards are indicated at the left of the panel. The prominent band in lane 8 (approximately 30 kDa) corresponds to the full length DKK2 protein.

FIG. 2 is a photograph of an SDS-PAGE gel/western analysis showing 1M salt washes from DKK2 expressing cells. DKK2 expression was assessed using an ant-DKK2 rabbit polyclonal antibody that recognizes the C2 domain of DKK2. Molecular weights in kDa of gel standards are indicated at the left of the panel. Calculated molecular weights of test constructs are listed at the right the lane legend.

FIG. 3 is a photograph of an SDS-PAGE gel stained with Coomassie blue showing an expression test of DKK2-C2. Molecular weights in kDa of gel standards are indicated at the left of the panel.

FIG. 4 is a photograph of an SDS-PAGE gel stained with Coomassie blue showing denatured DKK2-C2 purified by nickel chromatography.

FIG. 5 is a photograph of an SDS-PAGE gel stained with Coomassie blue showing the results of using refolding Buffer C for testing refolding conditions to generate monomeric hDKK2-C2.

FIG. 6 shows the analysis of the refolded sample by size exclusion chromatography (SEC) top panel and by SDS-PAGE under reducing and non-reducing conditions. The elution profile of SEC molecular weight standards is shown in the bottom panel.

FIG. 7 shows the analysis of the refolded and purified DKK2-C2 sample by SEC and by SDS-PAGE under non-reducing conditions.

FIG. 8 is a schematic representation of the DKK2-C2 construct used in Example 2.

FIG. 9 are photographs of SDS-PAGE gels comparing DKK2-C2 preparations Sample 1 and 2 produced in E. coli.

FIG. 10 is a schematic diagram summarizing the different Fc fusion designs studied in Example 3.

FIG. 11 shows the results of purification of Fc fusions on Protein A Sepharose. SDS-PAGE analysis of elution fractions was stained with Simply blue. Under reducing conditions the prominent band at 38 kDa is consistent with the molecular mass of the intact fusion protein and the band at 70 kDa under non-reducing conditions is consistent with the molecular weight of the dimer, which is characteristic of an Fc fusion protein where 2 monomers are held together by interchain disulfides in the hinge region of the Fc. Visible in the analysis is a prominent clipped form and high molecular weight aggregates seen under non reducing conditions.

FIG. 12 shows the results of analysis of Protein A eluate by analytical size exclusion chromatography (top panel). The elution profile of SEC molecular weight standards is shown in the bottom panel. In contrast to the SDS-PAGE profile, SEC revealed that 80% of the protein or more was aggregated and eluted with molecular weight of greater than 640 kDa.

FIG. 13 shows the results of purification of Protein A eluate on Heparin Sepharose. Absorbance (blue) and conductivity (green) are shown in the column chromatogram. Column fractions containing absorbance at 280 nm were subjected to SDS-PAGE and stained with Simply blue.

FIG. 14 is an analysis of cation exchange elution fractions from FIG. 13 by analytical size exclusion chromatography. Top panel shows the elution profile of gel filtration markers: A-void volume, B-640 kDa, C-150 kDa, D-44 kDa, E-17 kDa, F˜1 kDa.

FIG. 15 is a graphical representation of the analysis of the activity of Fc fusion, HSA fusion, and DKK2 alone protein samples in the Super Top Flash Assay. From bottom to top at the 50 nM point: HSA-DKK2; DKK2 (R&D); DKK2-HSA; DKK2C2-Fc; DKK2-Fc; and Fc-DKK2C2.

FIG. 16 is a photograph of an SDS-PAGE gel stained with Simply blue showing the results of purification of Fc-DKK2 C2 samples on Protein A Sepharose.

FIG. 17 is an analysis of Protein A eluates shown in FIG. 16 by analytical size exclusion chromatography. Top panel shows the elution profile of gel filtration markers and bottom panel shows the elution profile of free Fc alone.

FIG. 18 provides the results of the fractionation of ACE 476 on a Q-Sepharose column. Absorbance (blue) and conductivity (green) measurements are shown in the column chromatogram. Column fractions indicated were subjected to SDS-PAGE and stained with Simply blue (left panel) or subjected to SDS-PAGE/western and analyzed using an ant-DKK2 rabbit polyclonal antibody that recognizes the C2 domain of DKK2 (right panel).

FIG. 19 shows the results of fractionation of ACE 476 enriched sample from the Q-Sepharose column (FIG. 18) on Phenyl Sepharose followed by capture on Q-Sepharose. Samples were subjected to SDS-PAGE and stained with Simply Blue (left panel) or SDS-PAGE/western analysis using an ant-DKK2 rabbit polyclonal antibody that recognizes the C2 domain of DKK2 (right panel).

FIG. 20 shows the results of the fractionation of ACE 475 on a Q-Sepharose column. Samples were subjected to SDS-PAGE and stained with Simply blue (left panel) or SDS-PAGE/western analysis using an ant-DKK2 rabbit polyclonal antibody that recognizes the C2 domain of DKK2 (right panel).

FIG. 21 is a schematic representation of the HSA-DKK2 full length (C1+C2) construct.

FIG. 22 shows the results of purification of ACE 448 HSA-DKK2 C1+C2 on CaptureSelect HSA and analysis by SDS-PAGE/Western (left panel stained with Simply blue, right panel visualized using an ant-DKK2 rabbit polyclonal antibody that recognizes the C2 domain of DKK2).

FIG. 23 shows the results of purification of ACE 448 HSA-DKK2 C1+C2 on Heparin Sepharose and SDS-PAGE analysis of column fractions stained with Simply blue. Absorbance (blue) and conductivity (green) measurements are shown in the column chromatogram.

FIG. 24 is a graphical depiction of the analysis of the activity of samples in the Super Top Flash Assay.

FIG. 25 is a schematic representation of the ACE 449 DKK2 full length (C1+C2)-HSA construct.

FIG. 26 is a photograph of a gel showing the purification of ACE 449 DKK2 full length (C1+C2)-HSA on CaptureSelect™ HSA and its analysis by SDS-PAGE with samples stained with Simply blue.

FIG. 27 is a graphical depiction of the analysis of ACE 448 and ACE 449 by analytical size exclusion chromatography.

FIG. 28 shows schematic representations of HSA fusion constructs of the DKK2 C2 domain.

FIG. 29 is a photograph of the analysis of HSA-DKK2 C2 samples by SDS-PAGE stained with Simply blue.

FIG. 30 is a graphical depiction of the column chromatograms from the analysis of HSA-DKK2 C2 samples by analytical size exclusion chromatography. ACE 461 (top panel), ACE 463 (second panel), ACE 464, (third panel), ACE 465 (fourth panel), ACE 466 (fifth panel), HSA (sixth panel), and gel filtration molecular weight markers (bottom panel).

FIG. 31 is a graphical representation of the analysis of the activity of HSA-DKK2 C2 samples in the Super Top Flash Assay.

FIG. 32A is a graphical depiction of a pharmacokinetics comparison between HSA-DKK2C2 and DKK2C2. STF analysis is depicted of serum samples from mice dosed with 1.5 mpk HSA-DKK2C2, 10 mpk of HSA-DKK2C2, 0.2 mpk DKK2C2, or 2 mpk DKK2C2.

FIG. 32B is a graphical depiction of the analysis of the serum half-life of ACE 464 in rats. The dotted line denotes the limit of quantitation for the assay.

FIG. 33 is a graphical depiction of the analysis of ACE 511 and ACE 486 in the Super Top Flash Assay. Curves top to bottom at 1.0 nM concentration: HSA-DKK2 C2 464 (Old Stock); ACE486; ACE511; and HSA-DKK2 C2 464.

FIG. 34 is an alignment of the amino acid sequences of the C2 domain of DKK2, DKK1, and DKK4 from different species (m=mouse; h=human; x=Xenopus; r=rat; z=zebrafish) taken from Chen et al., J. Biol. Chem., 283(34):23364-23370 (2008). The dots indicate residues that are required for Lipoprotein receptor like proteins 5 and 6 (LRP5/6) binding. The paired cysteines are indicated by brackets.

FIG. 35 is a photograph of a SDS-PAGE gel stained with Simply blue examining supernatant from CHO cells expressing HSA-huDKK2 C2 heparin-binding mutants. Lane1: molecular weight marker; Lanes 2 and 14: pACE464-5 μg purified wild-type HSA-huDKK2 C2; lane 3: pACE464-2.5 μg purified wild-type; lane 4: pBKM225-K220N; lane 5: pBKM226-K220E; lane 6: pBKM227-H223E; lane 7: pBKM228-K216E/H223E; lane 8: pBKM229-K216E/K220E; lane 9: pBKM230-R197E; lane 10: pBKM231-K202E; lane 11: pBKM232-K216S/H223T; lane 12: pBKM233-K216S/K220S; lane 15: pACE502-R185N; lane 16: pACE503-K202E/K220E; lane 17: pACE504-K240E/K243E; lane 18: pACE505-K216E/K250E; lane 19: pACE506-K250E; lane 20: pACE507-S248N/K250S.

FIG. 36 is a photograph of a SDS-PAGE gel stained with Simply blue examining purified HSA-huDKK2 C2 heparin-binding mutants. Lane1: molecular weight marker; Lane 2: pACE464-wild-type; lane 3: pBKM225-K220N; lane 4: pBKM226-K220E; lane 5: pBKM227-H223E; lane 6: pBKM228-K216E/H223E; lane 7: pBKM229-K216E/K220E; lane 8: pBKM230-R197E; lane 9: pBKM231-K202E; lane 10: pBKM232-K216S/H223T; lane 11: pBKM233-K216S/K220S; lane 12: pACE502-R185N; lane 13: pACE504-K240E/K243E; lane 14: pACE505-K216E/K250E; lane 15: pACE506-K250E pH 5.5 purification; lane 16: pACE506-K250E pH 6.5 purification; lane 17: pACE507-S248N/K250S pH 5.5 purification, 300 mM elution fractions 2 and 3; lane 18: pACE507-S248N/K250S pH 5.5 purification, 300 mM elution fractions 4 and 5; lane 19: pACE507-S248N/K250S pH 6.5 purification.

FIG. 37 is a graphical depiction of analytical SEC profiles of HSA-huDKK2 C2 mutants. Elution profiles, monitoring absorbance at 280 nm (y-axis: absorbance units (AU)) for wild type and HSA-huDKK2 C2 mutants, from a 5 ml Superdex 200 column. The percent purity of all mutants was greater than 86%. The broadening and shift of variant ACE507 S248N/K250S (pH 5.5 purification, 300 mM NaCl elution fractions 2 and 3) is consistent with glycosylation.

FIG. 38 is a photograph of a native PAGE gel stained with Simply blue examining purified HSA-huDKK2 C2 heparin-binding mutants. Lane 1: pACE464-wild-type; lane 2: pBKM225-K220N; lane 3: pBKM226-K220E; lane 4: pBKM227-H223E; lane 5: pBKM228-K216E/H223E; lane 6: pBKM229-K216E/K220E; lane7: pBKM230-R197E; lane 8: pBKM231-K202E; lane 9: pBKM232-K216S/H223T; lane 10: pBKM233-K216S/K220S; lane 11: pACE502-R185N; lane 12: pACE504-K240E/K243E; lane 13: pACE505-K216E/K250E; lane 14: pACE506-K250E pH 5.5 purification; lane 15: pACE506-K250E pH 6.5 purification; lane 16: pACE507-S248N/K250S pH 5.5 purification, 300 mM elution fractions 2 and 3; lane 17: pACE507-S248N/K250S pH 5.5 purification, 300 mM elution fractions 4 and 5; lane 18: pACE507-S248N/K250S pH 6.5 purification.

FIG. 39 is a graphical depiction of the results of Heparin sepharose chromatography of selected HSA-huDKK2-C2 mutants. Elution profiles of seven selected mutants (BKM229: K216E/K220E; ACE505: K216E/K250E; BKM228: K216E/H223E; ACE504: K240E/K243E; BKM226: K220E; BKM227: H223E; BKM231: K202E) and wild-type HSA-huDKK2 C2, from a 1 ml heparin sepharose column over a linear sodium chloride gradient to 1M. The mutants binding heparin sepharose most weakly shared K216E mutation.

FIG. 40 includes graphical depictions of the results of heparin-biotin ELISA with selected HSA-huDKK2 C2 mutants. Titrations curve for biotin-heparin binding to HSA-huDKK2 mutants (comparable binders to wild type: top graph; weak heparin binders: bottom graph), plated at 15 μg/ml. Detection was with streptavidin-horseradish peroxidase after a 10-minute incubation. Eight mutants were found to bind monomeric heparin-biotin substantially less well than wild type.

FIG. 41 is a graphical depiction of the differential scanning fluorimetry (DSF) of selected HSA-huDKK2 C2 mutants. Thermal denaturation profiles of six selected mutants (key below based on curve position between 72 and 75° C.) (BKM229: K216E/K220E (third from bottom); ACE505: K216E/K250E (second from bottom); BKM228: K216E/H223E (third from top); ACE504: K240E/K243E (bottom); ACE506: K250E (third from bottom); BKM233: K216S/K220S (second from top); and wild-type HSA-huDKK2 C2 (top), from 25° C. to 95° C.

FIG. 42 are graphical depictions of HSA-huDKK2 C2 mutant competition with YW211.31.57 hu IgG1 agly anti-Lipoprotein receptor like protein 6 (LRP6) monoclonal antibody for binding to LRP6. LRP6 binding curve titrations for wild-type HSA-huDKK2 C2 (ACE464) and both heparin and LRP6 binding mutants (BKM195: H198A/K205A; BKM199: R230A), following competition with anti-LRP6 monoclonal antibody.

FIG. 43 is a bar graph depicting the pharmacokinetic analysis of heparin binding mutants in mice.

FIG. 44 is a series of graphs providing the results of the assessment of canonical Wnt3 inhibition by HSA-DKK2C2 heparin mutant constructs. HSA-DKK2C2 mutant constructs were tested in Wnt3a stimulated Super TopFlash (STF) cells to assess their ability to inhibit canonical Wnt signaling. STF cells were stimulated with no Wnt3a, Wnt3a alone, or Wnt3a plus HSA-DKK2C2 constructs. All data is shown relative to no Wnt3a stimulation. In the first panel, the top curve at position 1000 nM corresponds to ACE503; the second from top curve corresponds to ACE506; the third from top curve corresponds to ACE502; and the bottom curve corresponds to BKM233. In the second panel, the top curve at position 1000 nM corresponds to BKM229; the second from top curve corresponds to ACE505; the third from top curve corresponds to BKM228; the fourth from top curve corresponds to ACE504; and the bottom curve corresponds to ACE464. In the third panel, the top curve at position 1000 nM corresponds to BKM231; the second from top curve corresponds to ACE464; the third from top curve corresponds to ACE468; and the bottom curve corresponds to BKM232. In the fourth panel, the top curve at position 10 nM corresponds to ACE507; the second from top curve corresponds to BKM227; and the bottom curve corresponds to ACE464. In the fifth panel, the top curve at position 10 nM corresponds to BKM225; the second from top curve corresponds to BKM226; and the bottom curve corresponds to ACE464.

FIG. 45 is a bar graph providing the results of an assessment of phosphoLRP6 inhibition by HSA-DKK2C2 heparin mutant constructs. HSA-DKK2C2 mutant constructs were tested in Wnt3a stimulated Super TopFlash (STF) cells to assess their ability to inhibit pLRP6. STF cells were stimulated with no Wnt3a, Wnt3a alone, or Wnt3a plus HSA-DKK2C2 constructs. The ratio of pLRP6/LRP6 is normalized to β-actin loading controls, no Wnt3a stimulation, and displayed as a proportion of Wnt3a treatment alone. The key for the four bars for each construct is as follows: the left most bar corresponds to 0 nM; the second from left bar corresponds to 250 nM; the third from left bar corresponds to 500 nM; and the fourth from left bar corresponds to 1000 nM.

FIG. 46 is a schematic diagram showing conformational shifts between DKK2-C2(2JTK.pdb, white) and DKK1-C2 (3S8V.pdb, dark gray) structures; residue numbers are for DKK2-C2 (open) and DKK1-C2 (in parentheses). A number of basic residues undergo large conformational shifts between the two structures, such as H223(229), K220(226), or R218(224). As these residues form different charged patches based on their different backbone conformations, mutants were designed based on either conformation.

FIG. 47 is a schematic representation showing the location of basic patch #1 on the surface of DKK2-C2 (2JTK.pdb).

FIG. 48 is a schematic representation showing the location of basic patch #2 on the surface of DKK2-C2 (2JTK.pdb).

FIG. 49 is a schematic representation showing the location of the basic patch on the surface of DKK1-C2 (3S8V.pdb).

FIG. 50 are graphs representing comparison of binding to human LRP6 by HSA fusions of full length DKK2 (ACE 448), DKK2-C2 (ACE 464), reengineered DKK2-C2 (ACE 486 and ACE511), and non-PEGylated and PEGylated versions of untagged DKK2-C2 from E. coli, following competition with anti-LRP6 antibody.

### DETAILED DESCRIPTION

This disclosure is based, at least in part, on the unexpected discovery that the choice of fusion partner for a DKK2 polypeptide significantly affects the expression level, aggregation, disulfide scrambling, proteolytic lability, and activity of the DKK2 polypeptide. Human serum albumin (HSA) was identified as a highly effective fusion partner for DKK2 polypeptides. It was also discovered that deletion of the propeptide sequence of HSA can reduce heterogeneity of HSA-DKK2 fusion polypeptides. This disclosure also relates to the discovery that substitution of selected amino acid residues in DKK2 decreases heparin binding by variant DKK2 polypeptides.

### DKK2 Fusion Polypeptides

Based on a careful and extensive analysis of different strategies to augment DKK2 as a protein therapeutic, it was surprisingly discovered that the choice of fusion partner for a DKK2 polypeptide significantly affects the properties (e.g., expression, stability, or activity) of the DKK2 polypeptide. Fusion platforms with excellent pharmaceutical properties such as His, Fc, and XTEN were tested as fusion partners for DKK2 polypeptides. Untagged and His-tagged versions of full length DKK2 and cysteine rich domain 2 of DKK2 (DDK2-C2) polypeptides were found to have low expression and were highly aggregated. Fc tagged versions of full length DKK2 and DKK2-C2 polypeptides showed good levels of expression; however, there was clipping between the Fc polypeptide and the DKK2 polypeptide and the Fc-DKK2 fusion protein tended to aggregate. XTEN tagged versions of full length DKK2 and DDK2-C2 polypeptides expressed at moderate levels, but the expressed product was heterogeneous and exhibited poor recovery during purification.

In striking contrast, human serum albumin (HSA)-DKK-C2 fusion polypeptides showed high levels of expression and exhibited reduced proteolytic lability. Human serum albumin has many desirable pharmaceutical properties. These include: a serum half-life of 19-20 days; solubility of about 300 mg/mL; good stability; ease of expression; no effector function; low immunogenicity; and circulating serum levels of about 45 mg/mL. The crystal structure of HSA without and with ligands, including biologically important molecules such as fatty acids and drugs, or complexed with other proteins is well-known in the art. See, e.g., Universal Protein Resource Knowledgebase P02768; He et al., Nature, 358:209-215 (1992); Sugio et al., Protein Eng., 12:439-446 (1999). According to X-ray crystallographic studies of HSA, this polypeptide forms a heart-shaped protein with approximate dimensions of 80×80×80 Å and a thickness of 30 Å. It has about 67% α-helix but no β-sheet and can be divided into three homologous domains (I-III). Each of these three domains is comprised of two subdomains (A and B). The A and B subdomains have six and four α-helices, respectively, connected by flexible loops. The principal regions of ligand binding to human serum albumin are located in cavities in subdomains IIA and IIIA, which are formed mostly of hydrophobic and positively charged residues and exhibit similar chemistry. All but one of the 35 cysteine residues in the molecule are involved in the formation of 17 stabilizing disulfide bonds. The amino acid sequence as well as the structures of bovine, horse, rabbit, equine and leporine albumins are known. See, e.g., Majorek et al., Mol. Immunol., 52:174-182 (2012); Bujacz, Acta Crystallogr. D Biol. Crystallogr., 68:1278-1289 (2012). Numerous genetic variants of human serum albumin are well-known in the art. See, e.g., The Albumin Website maintained by the University of Aarhus, Denmark and the University of Pavia, Italy at albumin.org/genetic-variants-of-human-serum-albumin and albumin.org/genetic-variants-of-human-serum-albumin-reference-list.

In one embodiment, a human serum albumin used in the DKK-C2 fusions described herein comprises or consists of the amino acid sequence set forth below:

(SEQ ID NO: 50)
DARKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVIVICTAFHDNEETFLKKYLYEIARRHPY
FYAPELLFFAKRYKAAFTECCQAADKAACLLPKLDELRDEGKASSAKQRL
KCASLQKFGERAFKAWAVARLSQRFPKAEFAEVSKLVTDLTKVHTECCHG
DLLECADDRADLAKYICENQDSISSKLKECCEKPLLEKSHCIAEVENDEM
PADLPSLAADFVESKDVCKNYAEAKDVFLGMFLYEYARRHPDYSVVLLLR
LAKTYETTLEKCCAAADPHECYAKVFDEFKPLVEEPQNLIKQNCELFEQL
GEYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETYV
PKEFNAETFTFHADICTLSEKERQIKKQTALVELVKHKPKATKEQLKAVM
DDFAAFVEKCCKADDKETCFAEEGKKLVAASQAALGL

In another embodiment, a human serum albumin used in the DKK-C2 fusions described herein is a HSA variant has an amino acid sequence that is at least 85%, at least 90%, at least 91%, at least 92%, at least 93%, at least 94%, at least 95%, at least 96%, at least 97%, at least 98%, or at least 99% identical to the amino acid sequence set forth in SEQ ID NO:50. Percent identity between amino acid sequences can be determined using the BLAST 2.0 program. Sequence comparison can be performed using an ungapped alignment and using the default parameters (Blossom 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

In certain embodiments, the human serum albumin used in the DKK2-C2 fusions described herein is a HSA variant that may have N and/or C-terminal deletions in the sequence of SEQ ID NO:50 (e.g., 1-10, 1-9, 1-8, 1-7, 1-6, 1-5, 1-4, 1-3, 1-2, 1, 2, 3, 4, 5, 6, 7, 8, 9, or 10 consecutive amino acids at the N- and/or C-terminal may be deleted). In some instances, the HSA variant has the same or substantially the same desirable pharmaceutical properties of HSA having the amino acid sequence of SEQ ID NO:50 (e.g., a serum half-life of 19-20 days; solubility of about 300 mg/mL; good stability; ease of expression; no effector function; low immunogenicity; and/or circulating serum levels of about 45 mg/mL). In some instances, the HSA used as the fusion partner is a genetic variant of HSA. In some instances, the HSA variant is any one of the 77 variants disclosed in Otagiri et al, 2009, Biol. Pharm. Bull. 32(4), 527-534 (2009). In certain embodiments, the HSA used as the fusion partner for the DKK2 polypeptides is a mutated version of HSA that has improved affinity for the neonatal Fc receptor (FcRn) relative to the HSA of SEQ ID NO:50 (see e.g., U.S. Pat. Nos. 9,120,875; 9,045,564; 8,822,417; 8,748,380; Sand et al., Front. Immunol., 5:682 (2014); Andersen et al., J. Biol. Chem., 289(19):13492-502 (2014); Oganesyan et al., J. Biol. Chem., 289(11):7812-24 (2014); Schmidt et al., Structure, 21(11):1966-78 (2013); WO 2014/125082A1; WO 2011/051489, WO2011/124718, WO 2012/059486, WO 2012/150319; WO 2011/103076; and WO 2012/112188, all of which are incorporated by reference herein). In certain instances, the HSA mutant is the E505G/V547A mutant. In certain instances, the HSA mutant is the K573P mutant. Such HSA mutants that HSA that have improved affinity for FcRn can be used to increase the half-life of a DKK2-C2 fusion polypeptide or further increase the serum half-life of a DKK2-C2 heparin binding mutant disclosed herein.

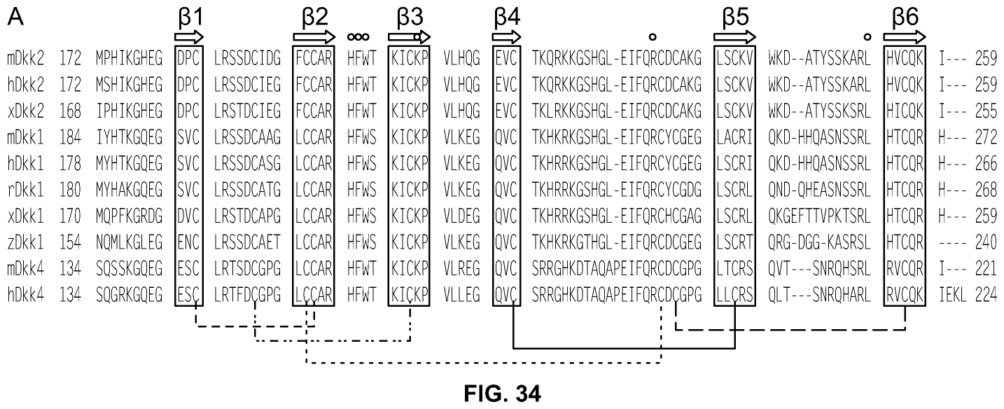

The HSA fusion polypeptides comprise a DKK2-C2 polypeptide. FIG. 34 provides an alignment of the amino acid sequences of the C2 domain of DKK2, DKK1, and DKK4 from different species (e.g., mouse, human, Xenopus, rat, and zebrafish). In one embodiment, the DKK2-C2 polypeptide comprises or consists of the amino acid sequence set forth below: HIKGHEGDPCLRSSDCIEGFCCARHFWTKICKPVLHQGEVCTKQRKKGSHGLEIFQRCD CAKGLSCKVWKDATYSSKARLHVCQKI (SEQ ID NO:51). In certain embodiments, the DKK2-C2 polypeptide comprises or consists of the amino acid sequence set forth below: MSHIKGHEGDPCLRSSDCIEGFCCARHFWTKICKPVLHQGEVCTKQRKKGSHGLEIFQR CDCAKGLSCKVWKDATYSSKARLHVCQKI (SEQ ID NO:2). In other embodiments, the DKK2-C2 polypeptide comprises or consists of the amino acid sequence set forth below:

(SEQ ID NO: 94)
MPHIKGHEGDPCLRSSDCIEGFCCARHFWTKICKPVLHQGEVCTKQRKKG
SHGLEIFQRCDCAKGLSCKVWKDATYSSKARLHVCQKI

In other embodiments, the DKK2-C2 polypeptide comprises or consists of an amino acid sequence that is at least 85%, at least 90%, at least 91%, at least 92%, at least 93%, at least 94%, at least 95%, at least 96%, at least 97%, at least 98%, or at least 99% identical to amino acid sequence set forth in SEQ ID NO:51, SEQ ID NO:2, or SEQ ID NO:93. In one embodiment, the DKK2-C2 polypeptide comprises or consists of an amino acid sequence that is at least 85%, at least 90%, at least 91%, at least 92%, at least 93%, at least 94%, at least 95%, at least 96%, at least 97%, at least 98%, or at least 99% identical to amino acid sequence set forth in SEQ ID NO:51. In certain instances, the DKK2-C2 polypeptide that is fused to HSA binds to human Lipoprotein receptor like protein 6 (LRP6) (e.g., with the same or substantially the same affinity as compared to a DKK-C2 polypeptide comprising or consisting of the amino acid sequence of SEQ ID NO:51). Methods of assessing binding between a receptor and another protein are well known in the art. Example 18 provides one way of examining binding to LRP6. In certain instances, the DKK2-C2 polypeptide that is fused to HSA shows reduced binding to heparin compared to a DKK-C2 polypeptide comprising or consisting of the amino acid sequence of SEQ ID NO:51. Examples 15 and 16 illustrate two different ways of examining whether a DKK2-C2 polypeptide binds to heparin. In certain instances, the DKK2-C2 polypeptide that is fused to HSA reduces Wnt induction (compared to a DKK-C2 polypeptide comprising or consisting of the amino acid sequence of SEQ ID NO:51) in a cell based reporter assay (e.g., Super Top Flash assay). In certain instances, the DKK2-C2 polypeptide that is fused to HSA is effective in promoting repair in a renal ischemia reperfusion injury model (e.g., decrease in tubule injury; improvement in renal function). In some cases, the DKK2-C2 polypeptide that is fused to HSA shows the same or substantially the same effectiveness in promoting repair in a renal ischemia reperfusion injury model as the DKK-C2 polypeptide comprising or consisting of the amino acid sequence of SEQ ID NO:51.

Provided herein are polypeptides comprising a first amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:50 that is directly linked or linked via a linker to a second amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:51. In certain embodiments, the polypeptide comprises a first amino acid sequence that is at least 95% identical to the amino acid sequence set forth in SEQ ID NO:50 and which is directly linked or linked via a linker to a second amino acid sequence that is at least 95% identical to the amino acid sequence set forth in SEQ ID NO:51. In a specific embodiment, the polypeptide comprises a first amino acid sequence and comprises a second amino acid sequence, wherein the first amino acid sequence is 100% identical to the amino acid sequence set forth in SEQ ID NO:50 and the second amino acid sequence is 100% identical to the amino acid sequence set forth in SEQ ID NO:51, and wherein and the first amino acid sequence is directly linked or linked via a linker to the second amino acid sequence.

There is no particular limitation on the linkers that can be used in the constructs described above. In some embodiments, the linker is a peptide linker. Any arbitrary single-chain peptide comprising about one to 25 residues (e.g., 2, 3, 4, 5, 6, 7, 8, 9, 10, 11, 12, 13, 14, 15, 16, 17, 18, 19, 20 amino acids) can be used as a linker. In certain instances, the linker contains only glycine and/or serine residues. Examples of such peptide linkers include: Gly; Ser; Gly Ser; Gly Gly Ser; Ser Gly Gly; Ala Ala; Ala Ala Ala; Gly Gly Gly Ser (SEQ ID NO:52); Ser Gly Gly Gly (SEQ ID NO:53); Gly Gly Gly Gly Ser (SEQ ID NO:54); Ser Gly Gly Gly Gly (SEQ ID NO:55); Gly Gly Gly Gly Gly Ser (SEQ ID NO:56); Ser Gly Gly Gly Gly Gly (SEQ ID NO:57); Gly Gly Gly Gly Gly Gly Ser (SEQ ID NO:58); Ser Gly Gly Gly Gly Gly Gly (SEQ ID NO:59); (Gly Gly Gly Gly Ser (SEQ ID NO:54)n, wherein n is an integer of one or more; and (Ser Gly Gly Gly Gly (SEQ ID NO:55)n, wherein n is an integer of one or more. In other embodiments, the linker peptides are modified such that the amino acid sequence GSG (that occurs at the junction of traditional Gly/Ser linker peptide repeats) is not present. For example, the peptide linker comprise an amino acid sequence selected from the group consisting of: (GGGXX)nGGGGS (SEQ ID NO:60) and GGGGS(XGGGS)n (SEQ ID NO:61), where X is any amino acid that can be inserted into the sequence and not result in a polypeptide comprising the sequence GSG, and n is 0 to 4. In one embodiment, the sequence of a linker peptide is (GGGX1X2)nGGGGS and X1 is P and X2 is S and n is 0 to 4 (SEQ ID NO:62). In another embodiment, the sequence of a linker peptide is (GGGX1X2)nGGGGS and X1 is G and X2 is Q and n is 0 to 4 (SEQ ID NO:63). In another embodiment, the sequence of a linker peptide is (GGGX1X2)nGGGGS and X1 is G and X2 is A and n is 0 to 4 (SEQ ID NO:64). In yet another embodiment, the sequence of a linker peptide is GGGGS(XGGGS)n, and X is P and n is 0 to 4 (SEQ ID NO:65). In one embodiment, a linker peptide of the invention comprises or consists of the amino acid sequence (GGGGA)2GGGGS (SEQ ID NO:66). In another embodiment, a linker peptide comprises or consists of the amino acid sequence (GGGGQ)2GGGGS (SEQ ID NO:67). In yet another embodiment, a linker peptide comprises or consists of the amino acid sequence (GGGPS)2GGGGS (SEQ ID NO:68). In a further embodiment, a linker peptide comprises or consists of the amino acid sequence GGGGS(PGGGS)2 (SEQ ID NO:69).

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), di sulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES). Since HSA contains a single free cysteine that can be used for targeted cross-linking, heterobifunctional cross-linkers that target this site can also be used. Examples of heterobifunctional cross-linking agents that are available on the market include, but are not limited to, GMBS, MBS, LC-SPDP, SMCC, SMPB, and SMPT.

### Variant DKK2-C2 Polypeptides

This disclosure also provides several variant polypeptides of the cysteine rich domain 2 (C2) of DKK2. These variants include mutations (e.g., substitutions, insertions, and/or deletions) at one or more positions within C2. The mutated C2 domain may be in the context of a full length DKK2 protein or as part of a fusion protein of a DKK2 polypeptide or fragment thereof (e.g., human serum albumin-DKK2, human serum albumin-DKK2-C2 fusion). In certain embodiments, the fusion partner for the DKK2-C2 polypeptides is a HSA variant discussed above. In a specific embodiment, the HSA variant has improved affinity for FcRn relative to HSA of SEQ ID NO:50. In some embodiments, these variant DKK2-C2 polypeptides show reduced binding to heparin relative to a polypeptide comprising or consisting of the amino acid sequence set forth in SEQ ID NO:51. Heparan sulfate is a sulfated polysaccharide covalently part of proteoglycans found on the surface of most cells and mediates interactions between different proteins. Non-specific cell interactions through heparan sulfate decrease serum exposure of proteins resulting in reduced serum half-life. Mutations in DKK2 C2 were created to reduce or eliminate heparan sulfate binding so as to decrease non-specific cell interactions through heparan sulfate and thereby increase DKK2 C2 serum exposure.

Wild type human cysteine rich domain 2 of DKK2 (hu DKK2-C2) is 88 amino acids in length and has the following amino acid sequence:

(SEQ ID NO: 2)
MSHIKGHEGDPCLRSSDCIEGFCCARHFWTKICKPVLHQGEVCTKQRKKG
SHGLEIFQRCDCAKGLSCKVWKDATYSSKARLHVCQKI

The variant hu DKK2-C2 polypeptides can be at least 80%, at least 85%, at least 90%, at least 91%, at least 92%, at least 93%, at least 94%, at least 95%, at least 96%, at least 97%, at least 98%, or at least 99% identical to SEQ ID NO:2. In certain instances, the hu DKK2-C2 polypeptide (i.e., SEQ ID NO:2) can be truncated at the N-terminus to remove ten or fewer, nine or fewer, eight or fewer, seven or fewer, six or fewer, five or fewer, four or fewer, three or fewer, two, or one amino acid. In other instances, the hu DKK2-C2 polypeptide can be truncated at the C-terminus to remove three or fewer, two, or one amino acid. In yet other instances, the hu DKK2-C2 polypeptide can be truncated at both the N- and C-terminus to remove ten or fewer, nine or fewer, eight or fewer, seven or fewer, six or fewer, five or fewer, four or fewer, three or fewer, two, or one amino acid. An exemplary N-terminally truncated version of wild type hu DKK2-C2 is 86 amino acids in length and has the following amino acid sequence:

(SEQ ID NO: 51)
HIKGHEGDPCLRSSDCIEGFCCARHFWTKICKPVLHQGEVCTKQRKKGSH
GLEIFQRCDCAKGLSCKVWKDATYSSKARLHVCQKI

FIG. 34 provides an alignment of the wild type human, mouse, and Xenopus DKK2-C2 polypeptides with wild type human, mouse, rat, zebrafish, and Xenopus DKK1-C2 polypeptides and mouse and human DKK4-C2 polypeptides. This figure identifies important residues for the structure and function of this domain including the residues required for LRP5/6 binding, the six beta strands, and the cysteines that are paired in the C2 domain. This alignment of naturally occurring, bioactive forms of DKK polypeptides indicates specific exemplary residues (i.e., those that are not conserved among the different species) that can be substituted without eliminating bioactivity. The substitution may be with a conservative or non-conservative amino acid.

A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, Ile, Phe or Trp) is substituted for, or by, one having a smaller side chain (e.g., Ala, Ser) or no side chain (e.g., Gly).

A variant DKK2-C2 polypeptide can contain five or fewer, four or fewer, three or fewer, two or fewer, or five, four, three, two, or one amino acid substitution, relative to SEQ ID NO:2, at: (i) a serine residue at position 16; (ii) a glutamic acid residue at position 20; (iii) a glutamine residue at position 46; (iv) an alanine residue at position 80; and/or (v) a valine residue at position 84. In certain embodiments, the serine residue at position 16 may be substituted with a threonine or phenylalanine; and/or the glutamic acid residue at position 20 may be substituted with an aspartic acid, alanine, serine, threonine, or proline; and/or the glutamine residue at position 46 may be substituted with a leucine, histidine, or arginine; and/or the alanine residue at position 80 may be substituted with a serine; and/or the valine residue at position 84 may be substituted with an isoleucine or threonine. In specific embodiments, the serine residue at position 16 may be substituted with a threonine; and/or the glutamic acid residue at position 20 may be substituted with an aspartic acid; and/or the glutamine residue at position 46 may be substituted with a leucine; and/or the alanine residue at position 80 may be substituted with a serine; and/or the valine residue at position 84 may be substituted with an isoleucine. The above-referenced mutations in DKK2-C2 may be present in combination with other mutations such as those described below.

Disclosed herein are polypeptides that can have substitutions at one or more selected amino acid residues of the hu DKK2-C2 polypeptide. In some instances one or more (e.g., 1, 2, 3, 4) basic residues (e.g., lysine, arginine) of hu DKK2-C2 are replaced with an acidic residue (e.g., glutamic acid, aspartic acid) or an uncharged residue (e.g., serine, threonine). In other instances one or more (e.g., 1, 2, 3, 4) serine residues of DKK2-C2 are substituted with an asparagine residue. In some instances one or more (e.g., 1, 2, 3, 4) histidine residues of DKK2-C2 are substituted with glutamic acid or threonine. In one embodiment, a variant DKK2-C2 polypeptide contains an amino acid substitution, relative to SEQ ID NO:2, at one or more (e.g., 1, 2, 3, 4) of: (i) an arginine residue at one or more of positions 14 or 26, and/or (ii) a lysine residue at one or more of positions 31, 45, 49, 69, 72, or 79, and/or (iii) a histidine residue at position 52; and/or (iv) a serine residue at position 79. In certain embodiments, the amino acid substitution relative to SEQ ID NO:2, occurs at at least one (e.g., 1, 2, 3, 4) lysine residue at positions 45, 49, 69, 72, or 79. Additionally, the amino acid substitution relative to SEQ ID NO:2, may occur at a histidine residue at position 52. These substitutions may be non-conservative substitutions or conservative substitutions. In some embodiments, the substitution(s) reduce the basic charge of the DKK-C2 polypeptide. The theoretical isoelectric point (pI) of DKK2-C2 is 9.11. In some embodiments, the amino acid substitutions discussed herein can reduce the pI of the variant DKK2-C2 polypeptide below 9.11 (e.g., between 8.0 and 9.0; between 8 and 8.5; between 8.5 and 9.0; between 7.5 and 8.0; between 7.0 and 7.5). The C2 mutations discussed above can result in a variant DKK2-C2 polypeptide having reduced heparin binding ability relative to a wild type DKK2-C2 polypeptide. Heparin binding can be assessed by any method known in the art. For example, one could use the methods described in Examples 14 and 15 herein. The C2 mutations discussed above can also improve the pharmacokinetics of DKK2-C2 relative to the wild type DKK2-C2 polypeptide. This can be evaluated e.g., as shown in Example 19.

Exemplary variant DKK2-C2 polypeptides are disclosed in Table 1. Amino acid residues of the variant DKK2-C2 polypeptides that are mutated as compared to the corresponding wild type position are bolded.

TABLE 1
Exemplary Variant DKK2-C2 Polypeptides
(using numbering observed in the context of full length
DKK2 for position substituted)

| SEQ | Position | |
|---|---|---|
| ID NO | Substituted | Amino Acid Sequence |
| 70 | R185N | HIKGHEGDPCLNSSDCIEGFCCARHFWTKICKPVLHQGEVCTKQ |
| | | RKKGSHGLEIFQRCDCAKGLSCKVWKDATYSSKARLHVCQKI |
| 71 | R197E | HIKGHEGDPCLRSSDCIEGFCCAEHFWTKICKPVLHQGEVCTKQ |
| | | RKKGSHGLEIFQRCDCAKGLSCKVWKDATYSSKARLHVCQKI |
| 72 | K202E | HIKGHEGDPCLRSSDCIEGFCCARHFWTEICKPVLHQGEVCTKQ |
| | | RKKGSHGLEIFQRCDCAKGLSCKVWKDATYSSKARLHVCQKI |
| 73 | K216E | HIKGHEGDPCLRSSDCIEGFCCARHFWTKICKPVLHQGEVCTEQ |
| | | RKKGSHGLEIFQRCDCAKGLSCKVWKDATYSSKARLHVCQKI |
| 74 | K216S | HIKGHEGDPCLRSSDCIEGFCCARHFWTKICKPVLHQGEVCTSQ |
| | | RKKGSHGLEIFQRCDCAKGLSCKVWKDATYSSKARLHVCQKI |
| 75 | K220E | HIKGHEGDPCLRSSDCIEGFCCARHFWTKICKPVLHQGEVCTKQ |
| | | RKEGSHGLEIFQRCDCAKGLSCKVWKDATYSSKARLHVCQKI |
| 76 | K220N | HIKGHEGDPCLRSSDCIEGFCCARHFWTKICKPVLHQGEVCTKQ |
| | | RKNGSHGLEIFQRCDCAKGLSCKVWKDATYSSKARLHVCQKI |
| 77 | H223E | HIKGHEGDPCLRSSDCIEGFCCARHFWTKICKPVLHQGEVCTKQ |
| | | RKKGSEGLEIFQRCDCAKGLSCKVWKDATYSSKARLHVCQKI |
| 78 | K240E | HIKGHEGDPCLRSSDCIEGFCCARHFWTKICKPVLHQGEVCTKQ |
| | | RKKGSHGLEIFQRCDCAKGLSCEVWKDATYSSKARLHVCQKI |
| 79 | K243E | HIKGHEGDPCLRSSDCIEGFCCARHFWTKICKPVLHQGEVCTKQ |
| | | RKKGSHGLEIFQRCDCAKGLSCKVWEDATYSSKARLHVCQKI |
| 80 | S248N | HIKGHEGDPCLRSSDCIEGFCCARHFWTKICKPVLHQGEVCTKQ |
| | | RKKGSHGLEIFQRCDCAKGLSCKVWKDATYNSKARLHVCQKI |
| 81 | K250E | HIKGHEGDPCLRSSDCIEGFCCARHFWTKICKPVLHQGEVCTKQ |
| | | RKKGSHGLEIFQRCDCAKGLSCKVWKDATYSSEARLHVCQKI |
| 82 | K250S | HIKGHEGDPCLRSSDCIEGFCCARHFWTKICKPVLHQGEVCTKQ |
| | | RKKGSHGLEIFQRCDCAKGLSCKVWKDATYSSSARLHVCQKI |
| 83 | K202E | HIKGHEGDPCLRSSDCIEGFCCARHFWTEICKPVLHQGEVCTKQ |
| | K220E | RKEGSHGLEIFQRCDCAKGLSCKVWKDATYSSKARLHVCQKI |
| 84 | K216E | HIKGHEGDPCLRSSDCIEGFCCARHFWTKICKPVLHQGEVCTEQ |
| | K220E | RKEGSHGLEIFQRCDCAKGLSCKVWKDATYSSKARLHVCQKI |
| 85 | K216E | HIKGHEGDPCLRSSDCIEGFCCARHFWTKICKPVLHQGEVCTEQ |
| | K250E | RKKGSHGLEIFQRCDCAKGLSCKVWKDATYSSEARLHVCQKI |
| 86 | K216E | HIKGHEGDPCLRSSDCIEGFCCARHFWTKICKPVLHQGEVCTEQ |
| | H223E | RKKGSEGLEIFQRCDCAKGLSCKVWKDATYSSKARLHVCQKI |
| 87 | K216S | HIKGHEGDPCLRSSDCIEGFCCARHFWTKICKPVLHQGEVCTSQ |
| | H223T | RKKGSTGLEIFQRCDCAKGLSCKVWKDATYSSKARLHVCQKI |
| 88 | K240E | HIKGHEGDPCLRSSDCIEGFCCARHFWTKICKPVLHQGEVCTKQ |
| | K243E | RKKGSHGLEIFQRCDCAKGLSCEVWEDATYSSKARLHVCQKI |
| 89 | K216S | HIKGHEGDPCLRSSDCIEGFCCARHFWTKICKPVLHQGEVCTSQ |
| | K220S | RKSGSHGLEIFQRCDCAKGLSCKVWKDATYSSKARLHVCQKI |
| 90 | S248N | HIKGHEGDPCLRSSDCIEGFCCARHFWTKICKPVLHQGEVCTKQ |
| | K250S | RKKGSHGLEIFQRCDCAKGLSCKVWKDATYNSSARLHVCQKI |

In some embodiments, the variant DKK2-C2 polypeptides described above can bind to LRP5 and/or LRP6. Any method for detecting binding to LRP5/6 can be used to evaluate the biological activity a variant DKK-C2 polypeptide. For example, one could use the method described in Example 18 herein.

In certain embodiments, the variant DKK2-C2 polypeptides described above can inhibit the canonical Wnt signaling pathway. Inhibition of the canonical Wnt pathway be assessed, e.g., using cell based Wnt reporter assays described in Wu et al., Curr Biol., 10:1611-1614 (2000) and Li et al., J. Biol. Chem., 277:5977-81 (2002). In a specific embodiment, Wnt signaling can be evaluated using the Super Top Flash cell line as in Xu et al., Cell, 116:883-895 (2004). Another non-limiting method to assess Wnt signaling is to evaluate the phosphorylation of the LRP5/6 tail (Tamai et al., Mol. Cell., 13(1):149-56 (2004)). Yet another method to determine the effect of the variant DKK2-C2 polypeptides on Wnt signaling is to determine the levels of beta-catenin; most cells respond to Wnt signaling by an increase in the levels of beta-catenin.

In certain embodiments, the variant DKK2-C2 polypeptides described above can rescue Wnt-induced axis duplication during Xenopus development. This can be tested, e.g., as described in Brott and Sokol, Mol. Cell. Biol., 22:6100-10 (2002).

In some embodiments, the variant DKK2-C2 polypeptides described above promote repair in a renal ischemia reperfusion injury model. Methods of testing the ability of the variant DKK2-C2 polypeptides to promote repair in a renal ischemia reperfusion injury model can be as described in Lin et al., Proc. Natl. Acad. Sci. USA, 107(9): 4194-4199 (2010).

In addition to the specific amino acid substitutions identified herein, a variant DKK2-C2 polypeptide can also contain one or more (e.g., 1, 2, 3, 4) additions, substitutions, and/or deletions at other amino acid positions.

The DKK2-C2 variant polypeptides described above can be fused at either their N- or C-terminus to a polypeptide comprising HSA (SEQ ID NO:50) or an amino acid sequence that is at least 85%, at least 90%, at least 91%, at least 92%, at least 93%, at least 94%, at least 95%, at least 96%, at least 97%, at least 98%, or at least 99% identical to the amino acid sequence set forth in SEQ ID NO:50.

A DKK2-C2 polypeptide and/or a HSA-DKK2-C2 polypeptide can optionally also contain heterologous amino acid sequences in addition to a variant DKK2-C2 and/or HSA polypeptides. “Heterologous,” as used when referring to an amino acid sequence, refers to a sequence that originates from a source foreign to the particular host cell, or, if from the same host cell, is modified from its original form. Exemplary heterologous sequences include a heterologous signal sequence (e.g., native rat albumin signal sequence, a modified rat signal sequence, or a human growth hormone signal sequence) or a sequence used for purification of a variant DKK2-C2 polypeptide (e.g., a histidine tag).

### Nucleic Acids and Methods of Making Variant Polypeptides

This disclosure also encompasses nucleic acid encoding the HSA fusions of DKK2-C2, variant DKK2, variant DKK2-C2, and HSA fusions of the variant DKK2, and variant DKK2-C2 polypeptides described above. The nucleic acid can be inserted into vectors (e.g., expression vectors.

The nucleic acids encoding HSA fusions of DKK2-C2, variant DKK2, variant DKK2-C2, and HSA fusions of the variant DKK2, and variant DKK2-C2 polypeptides described above can be expressed in any desired host cell (e.g., bacterial cells, yeast cells, mammalian cells). In certain embodiments, the polypeptide is secreted from the host cell. In a specific embodiment, the host cell is a yeast cell. In some instances, a DKK2 polypeptide coding sequence (e.g., DKK2-C2 or a heparin binding mutant thereof) is fused to the HSA coding sequence, either to the 5′ end or 3′ end. This makes it possible to secrete the HSA-polypeptide fusion protein from yeast without the requirement for a yeast-derived pro sequence.

If the polypeptide is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., Nature, 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion. For production into the periplasm of E. coli, the pelB signal sequence (Lei et al., J. Bacteriol., 169:4379 (1987)) may be used as the signal sequence for secretion. For bacterial expression, calcium chloride methods or electroporation methods may be used to introduce the expression vector into the bacterial cell.

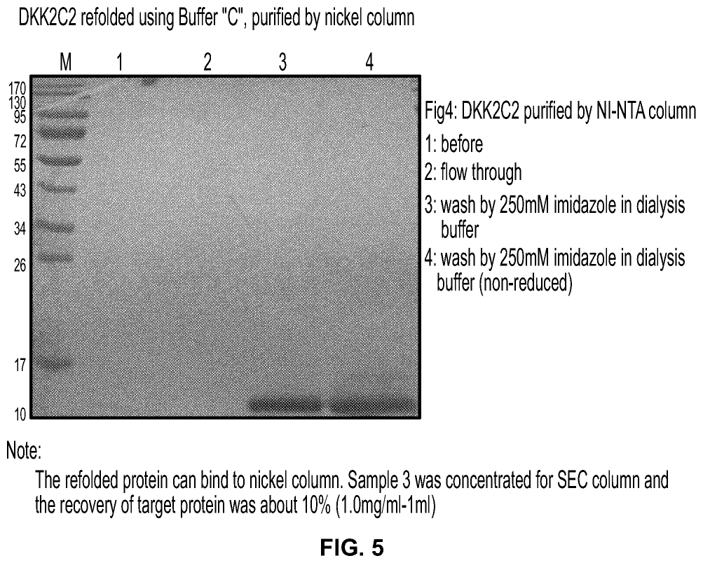

If the polypeptide is to be expressed in yeast cells (e.g., Saccharomyces cerevisiae, Saccharomyces italicus, Saccharomyces rouxii, Pichia pastoris, Pichia angusta, Pichia anomala, Pichia capsulate, Kluyveromyces lactis, or yeasts of the genera Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Borryoascus, Sporidiobolus, or Endomycopsis), the expression vector includes a promoter that drives expression of the polypeptide in the yeast cells and/or signal sequences effective for directing secretion in yeast. Suitable promoters for Saccharomyces include those associated with the PGK1 gene, GAL1 or GAL10 genes, CYC1, PHOS, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRB1 promoter, the GUT2 promoter, the GPD1 promoter, and hybrid promoters involving hybrids of parts of 5′ regulatory regions with parts of 5′ regulatory regions of other promoters or with upstream activation sites (e.g. the promoter described in EP-A-258 067). Suitable promoters for Pichia include AOX1, AOX2, MOX1 and FMD1. In some instances, the signal sequence is a yeast-derived signal sequence (e.g., one which is homologous to the yeast host). In some instances the HSA-polypeptide fusion molecule does not have a yeast-derived pro sequence between the signal sequence and the DKK2 polypeptide. The Saccharomyces cerevisiae invertase signal is a non-limiting example of a yeast-derived signal sequence. In certain embodiments, the yeast strains used to produce the polypeptides described herein are D88, DXY1 and BXP10. D88 [leu2-3, leu2-122, can1, pra1, ubc4] is a derivative of parent strain AH22his⁺ (also known as DB1: see, e.g. Sleep et al., Biotechnology, 8:42-46 (1990)). The strain contains a leu2 mutation which allows for auxotropic selection of 2 micron-based plasmids that contain the LEU2 gene. D88 also exhibits a derepression of PRB1 in glucose excess. The PRB1 promoter is normally controlled by two checkpoints that monitor glucose levels and growth stage. The promoter is activated in wild type yeast upon glucose depletion and entry into stationary phase. Strain D88 exhibits repression by glucose, but maintains induction upon entry into stationary phase. The PRA1 gene encodes a yeast vacuolar protease, YscA endoprotease A, that is localized in the ER. The UBC4 gene is in the ubiquitination pathway and is involved in targeting short lived and abnormal proteins for ubiquitin-dependent degradation. Isolation of this ubc4 mutation was found to increase the copy number of an expression plasmid in the cell and cause an increased level of expression of a desired protein expressed from the plasmid (see, e.g. WO 99/00504, hereby incorporated by reference in its entirety herein). DXY1, a derivative of D88, has the following genotype: [leu2-3, leu2-122, can1, pra1, ubc4, ura3::yap3]. In addition to the mutations isolated in D88, this strain also has a knockout of the YAP3 protease. This protease causes cleavage of mostly di-basic residues (RR, RK, KR, KK) but can also promote cleavage at single basic residues in proteins. Isolation of this yap3 mutation resulted in higher levels of full length HSA production (see. e.g., U.S. Pat. No. 5,965,386, and Kerry-Williams et al., Yeast, 14:161-169 (1998), hereby incorporated by reference in their entireties herein). BXP10 has the following genotype: leu2-3, leu2-122, can1, pra1, ubc4, ura3, yap3::URA3, lys2, hsp150::LYS2, pmr1::URA3. In addition to the mutations isolated in DXY1, this strain also has a knockout of the PMT1 gene and the HSP150 gene. The PMT1 gene is a member of the evolutionarily conserved family of dolichyl-phosphate-D-mannose protein 0-mannosyltransferases (Pmts). The transmembrane topology of Pmt1p suggests that it is an integral membrane protein of the endoplasmic reticulum with a role in O-linked glycosylation. This mutation serves to reduce/eliminate O-linked glycosylation of HSA fusions (see, e.g., WO00/44772, hereby incorporated by reference in its entirety herein). Studies revealed that the Hsp 150 protein is inefficiently separated from rHA by ion exchange chromatography. The mutation in the HSP150 gene removes a potential contaminant that has proven difficult to remove by standard purification techniques. See, e.g., U.S. Pat. No. 5,783,423, hereby incorporated by reference in its entirety herein. The desired polypeptide can be made in the yeast by transforming the yeast cells with a nucleic acid encoding the desired protein by any method known in the art. Examples of yeast plasmid vectors include pRS403 through pRS406 and pRS413-416 which are available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RPI. LEU2 and URA3. Plasmids pRS413-416 are Yeast Centromere plasmids (Ycps). Non-limiting examples of vectors for making HAS fusion proteins in yeast include pPPC0005, pScCHSA, pScNHSA, and pC4:HSA which are described in detail in Example 2 and FIG. 4 of U.S. Pat. No. 8,946,156 (incorporated by reference herein) and the pSAC35 vector which is described in Sleep et al., BioTechnology, 8:42 (1990) (incorporated by reference herein). The pPPC0005 plasmid can be used as the base vector into which polynucleotides encoding the DKK2 polypeptides (e.g., DKK2-C2 and heparin binding mutants thereof) described herein may be cloned to form HSA-fusions. It contains a PRB1 S. cerevisiae promoter, a fusion leader sequence, DNA encoding HAS, and an ADH1 S. cerevisiae terminator sequence. The sequence of the fusion leader sequence consists of the first 19 amino acids of the signal peptide of human serum albumin and the last five amino acids of the mating factor alpha 1 promoter (SLDKR (SEQ ID NO:93)), see EP-A-387 319 which is hereby incorporated by reference in its entirety herein. If the polypeptide is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the polypeptide, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

The polypeptide can also be expressed in human cells such as HEK-293 cells.

Variant DKK2 and variant DKK2-C2 polypeptides (and HSA-fusions thereof), can be constructed using any of several methods known in the art. One such method is site-directed mutagenesis, in which a specific nucleotide (or, if desired a small number of specific nucleotides) is changed in order to change a single amino acid (or, if desired, a small number of predetermined amino acid residues) in the encoded variant DKK2 or DKK2-C2 polypeptide. Many site-directed mutagenesis kits are commercially available. One such kit is the “Transformer Site Directed Mutagenesis Kit” sold by Clontech Laboratories (Palo Alto, Calif.).

DKK2, DKK2-C2, variant DKK2, and variant DKK2-C2 polypeptides and HSA-fusions thereof can be produced and isolated using methods well-known in the art. In some embodiments, variant DKK2 or variant DKK2-C2 polypeptides are produced by recombinant DNA techniques. For example, a nucleic acid molecule encoding a variant DKK2 or variant DKK2-C2 polypeptide can be inserted into a vector, e.g., an expression vector, and the nucleic acid can be introduced into a cell. Suitable cells include, e.g., mammalian cells (such as human cells or CHO cells), fungal cells, yeast cells, insect cells, and bacterial cells. When expressed in a recombinant cell, the cell is preferably cultured under conditions allowing for expression of a variant DKK2 or variant DKK2-C2 polypeptide. The variant DKK2 or variant DKK2-C2 polypeptide can be recovered from a cell suspension if desired. As used herein, “recovered” means that the mutated polypeptide is removed from those components of a cell or culture medium in which it is present prior to the recovery process. The recovery process may include one or more refolding or purification steps. Methods for isolation and purification commonly used for protein purification may be used for the isolation and purification of the polypeptides described herein, and are not limited to any particular method. Polypeptides may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column, Capture Select HSA, and Heparin Sepharose. Examples of columns using protein A column include Hyper D, POROS, and Sepharose FF (GE Healthcare Biosciences). The present disclosure also includes DKK2-C2 polypeptides and HSA-fusions thereof that are highly purified using these purification methods.

### Pharmaceutical Compositions

A variant DKK2 or DKK2-C2 polypeptide (or a HSA-fusion thereof) can be incorporated into a pharmaceutical composition containing a therapeutically effective amount of the polypeptide and one or more adjuvants, excipients, carriers, and/or diluents. Acceptable diluents, carriers and excipients typically do not adversely affect a recipient's homeostasis (e.g., electrolyte balance). Acceptable carriers include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride. Further details on techniques for formulation and administration of pharmaceutical compositions can be found in, e.g., REMINGTON'S PHARMACEUTICAL SCIENCES (Maack Publishing Co., Easton, Pa.).

Administration of a pharmaceutical composition containing a variant DKK2 or DKK2-C2 polypeptide (or a HSA-fusion thereof) can be systemic or local. Pharmaceutical compositions can be formulated such that they are suitable for parenteral and/or non-parenteral administration. Specific administration modalities include subcutaneous, intravenous, intramuscular, intraperitoneal transdermal, intrathecal, oral, rectal, buccal, topical, nasal, ophthalmic, intra-articular, intra-arterial, sub-arachnoid, bronchial, lymphatic, vaginal, and intra-uterine administration.

Formulations suitable for parenteral administration conveniently contain a sterile aqueous preparation of the variant DKK2 or DKK2-C2 polypeptide (or a HSA-fusion thereof), which preferably is isotonic with the blood of the recipient (e.g., physiological saline solution). Formulations may be presented in unit-dose or multi-dose form.

An exemplary formulation contains variant DKK2 or DKK2-C2 polypeptide (or a HSA-fusion thereof) described herein and the following buffer components: sodium succinate (e.g., 10 mM); NaCl (e.g., 75 mM); and L-arginine (e.g., 100 mM).

Formulations suitable for oral administration may be presented as discrete units such as capsules, cachets, tablets, or lozenges, each containing a predetermined amount of the variant DKK2 or DKK2-C2 polypeptide (or a HSA-fusion thereof); or a suspension in an aqueous liquor or a non-aqueous liquid, such as a syrup, an elixir, an emulsion, or a draught.

Therapeutically effective amounts of a pharmaceutical composition may be administered to a subject in need thereof in a dosage regimen ascertainable by one of skill in the art. For example, a composition can be administered to the subject, e.g., systemically at a dosage from 0.2 mg/kg to 200 mg/kg body weight of the subject, per dose. In another example, the dosage is from 0.5 mg/kg to 200 mg/kg body weight of the subject, per dose. In another example, the dosage is from 1 mg/kg to 100 mg/kg body weight of the subject, per dose. In a further example, the dosage is from 1 mg/kg to 50 mg/kg body weight of the subject, per dose. In another example, the dosage is from 2 mg/kg to 30 mg/kg body weight of the subject, per dose.

In order to optimize therapeutic efficacy, a variant DKK2 or DKK2-C2 polypeptide (or a HSA-fusion thereof) is first administered at different dosing regimens. The unit dose and regimen depend on factors that include, e.g., the species of mammal, its immune status, the body weight of the mammal. Typically, protein levels in tissue are monitored using appropriate screening assays as part of a clinical testing procedure, e.g., to determine the efficacy of a given treatment regimen.

The frequency of dosing for a variant DKK2 or DKK2-C2 polypeptide (or a HSA-fusion thereof) is within the skills and clinical judgement of physicians. Typically, the administration regime is established by clinical trials which may establish optimal administration parameters. However, the practitioner may vary such administration regimes according to the subject's age, health, weight, sex and medical status. The frequency of dosing may also vary between acute and chronic treatments for the disease or disorder. In addition, the frequency of dosing may be varied depending on whether the treatment is prophylactic or therapeutic.

### Methods of Treatment

Variant DKK2 or DKK2-C2 polypeptide (or a HSA-fusion thereof) described herein can be used for the treatment of a human subject having or at risk of developing fibrosis. There are several animal models of fibrosis that can be used to test efficacy of the polypeptides described herein (e.g., COL4A3−/−mice (e.g., Cosgrove et al., Amer. J. Path., 157:1649-1659 (2000), mice with Adriamycin-induced injury (Wang et al., Kidney Int'l., 58:1797-1804 (2000), db/db mice (Ziyadeh et al., PNAS USA, 97:8015-8020 (2000), mice with unilateral ureteral obstruction (Fogo et al., Lab Invest., 81:189A (2001))).

Variant DKK2 or DKK2-C2 polypeptide (or a HSA-fusion thereof) described herein can also be used for the treatment of a human subject having or at risk of developing acute kidney injury. Acute kidney injury (formerly known as acute renal failure) is a severe inflammation and damage of the kidney, which sometimes results in complete kidney failure. Acute kidney injury is characterized by the rapid loss of the kidney's excretory function and is typically diagnosed by the accumulation of end products of nitrogen metabolism (urea and creatinine) or decreased urine output, or both. It is the clinical manifestation of several disorders that affect the kidney acutely. Patients who have had acute kidney injury are at increased risk of developing chronic kidney disease. Acute kidney injury is a condition that is common in hospital patients and very common in critically ill patients. Hospital-acquired acute kidney injury affects approximately 2 million patients in the Western World. Thus, it poses a significant clinical problem that complicates the course of hospitalization and portends worse clinical outcomes for hospitalized patients. Acute kidney injury diagnoses are increasing in part because of an aging population, increased exposure to nephrotoxic drugs or infections in hospitals, as well as an increasing number of surgical interventions. Depending on the severity of kidney failure, the mortality rate ranges from 7% to as high as 80%, with an average of approximately 35%. Approximately 700,000 deaths in Europe, the US, and Japan each year are linked to this disease.

Acute kidney injury is commonly divided into two major categories based on the type of insult. The first category is ischemic acute kidney injury (alternatively referred to as kidney hypoperfusion) and the second category is nephrotoxic acute kidney injury. The former results from impaired blood flow (kidney hypoperfusion) and oxygen delivery to the kidney; whereas, the latter results from a toxic insult to the kidney. Both of these categories of insults can lead to a secondary condition called acute tubular necrosis.

The most common causes of ischemic acute kidney injury are intravascular volume depletion, reduced cardiac output, systemic vasodilatation, and renal vasoconstriction. Intravascular volume depletion can be caused by hemorrhage (e.g., following surgery, postpartum, or trauma); gastrointestinal loss (e.g., from diarrhea, vomiting, nasogastric loss); renal losses (e.g., caused by diuretics, osmotic diuresis, diabetes insipidus); skin and mucous membrane losses (e.g., burns, hyperthermia); nephrotic syndrome; cirrhosis; or capillary leak. Reduced cardiac output can be due to cardiogenic shock, pericardial disease (e.g., restrictive, constrictive, tamponade), congestive heart failure, valvular heart disease, pulmonary disease (e.g., pulmonary hypertension, pulmonary embolism), or sepsis. Systemic vasodilation can be the result of cirrhosis, anaphylaxis, or sepsis. Finally, renal vasoconstriction can be caused by early sepsis, hepatorenal syndrome, acute hypercalcemia, drug-related (e.g., norepinephrine, vasopressin, nonsteroidal anti-inflammatory drugs, angiotensin-converting enzyme inhibitors, calcineurin inhibitors), or use of a radiocontrast agent. The polypeptides described herein can be used to treat or reduce the symptoms or severity of acute kidney injury or other kidney injury caused by any of the above mentioned causes of ischemic acute kidney injury. In addition, the polypeptides described herein can be used to prevent the development of acute kidney injury or any other kidney injury following exposure to the above mentioned causes of ischemic acute kidney injury.

Nephrotoxic acute kidney injury is often associated with exposure to a nephrotoxin such as a nephrotoxic drug. Examples of nephrotoxic drugs include an antibiotic (e.g., aminoglycosides such as gentamicin), a chemotherapeutic agent (e.g., cis-platinum), a calcineurin inhibitor (e.g., tacrolimus, cyclosporine), cephalosporins such as cephaloridine, cyclosporin, pesticides (e.g., paraquat), environmental contaminants (e.g., trichloroethylene, dichloroacetylene), amphotericin B, puromcyin, aminonucleoside (PAN), a radiographic contrast agent (e.g., acetrizoate, diatrizoate, iodamide, ioglicate, iothalamate, ioxithalamate, metrizoate, metrizamide, iohexol, iopamidol, iopentol, iopromide, and ioversol), a non-steroidal anti-inflammatory, an anti-retroviral, an immunosuppressant, an oncological drug, or an ACE inhibitor. A nephrotoxin can be, for example, a trauma injury, a crush injury, an illicit drug, analgesic abuse, a gunshot wound, or a heavy metal. The polypeptides described herein can be used to treat or reduce the symptoms or severity of acute kidney injury or any other kidney injury caused by any of the above mentioned causes of nephrotoxic acute kidney injury.

In certain embodiments, the polypeptides described herein can be used to reduce the risk of, or prevent, development of acute tubular necrosis following exposure to an insult such as ischemia or nephrotoxins/nephrotoxic drugs. In certain embodiments, the polypeptides described herein can be used to treat or reduce the symptoms or severity of acute tubular necrosis following ischemia or exposure to nephrotoxins/nephrotoxic drugs.

In certain embodiments, the polypeptides described herein can be used to reduce the risk of, or prevent, a drop in glomerular filtration following ischemia or exposure to nephrotoxins/nephrotoxic drugs. In some embodiments, the polypeptides described herein can be used to prevent tubular epithelial injury and/or necrosis following ischemia or exposure to nephrotoxins/nephrotoxic drugs. In some embodiments, the polypeptides described herein can be used to decrease the microvascular permeability, improve vascular tone, and/or reduce inflammation of endothelial cells. In other embodiments, the polypeptides can be used to restore blood flow in the kidney following ischemia or exposure to nephrotoxins/nephrotoxic drugs. In further embodiments, the polypeptides described herein can be used to prevent chronic renal failure.

The polypeptides described herein can also be used to treat or prevent acute kidney injury resulting from surgery complicated by hypoperfusion. In certain specific embodiments, the surgery is one of cardiac surgery, major vascular surgery, major trauma, or surgery associated with treating a gunshot wound. In one embodiment, the cardiac surgery is coronary artery bypass grafting (CABG). In another embodiment, the cardiac surgery is valve surgery.

In some embodiments, the polypeptides described herein can be used to treat or prevent acute kidney injury following organ transplantation such as kidney transplantation or heart transplantation.

In some embodiments, the polypeptides described herein can be used to treat or prevent acute kidney injury following reduced effective arterial volume and kidney hypoperfusion.

In some embodiments, the polypeptides described herein can be used to treat or prevent acute kidney injury in a subject who is taking medication (e.g., an anticholinergic) that interferes with normal emptying of the bladder. In certain embodiments, the polypeptides described herein can be used to treat or prevent acute kidney injury in a subject who has an obstructed urinary catheter. In some embodiments, the polypeptides described herein can be used to treat or prevent acute kidney injury in a subject who is taking a drug that causes crystalluria. In some embodiments, the polypeptides described herein can be used to treat or prevent acute kidney injury in a subject who is taking a drug that causes or leads to myoglobinuria. In some embodiments, the polypeptides described herein can be used to treat or prevent acute kidney injury in a subject who is taking a drug that causes or leads to cystitis.

In some embodiments, the polypeptides described herein can be used to treat or prevent acute kidney injury in a subject who has benign prostatic hypertrophy or prostate cancer.

In some embodiments, the polypeptides described herein can be used to treat or prevent acute kidney injury in a subject who has a kidney stone.

In some embodiments, the polypeptides described herein can be used to treat or prevent acute kidney injury in a subject who has an abdominal malignancy (e.g., ovarian cancer, colorectal cancer).

In certain embodiments, the polypeptides described herein can be used to treat or prevent acute kidney injury, wherein sepsis does not cause or result in the acute kidney injury.

Acute kidney injury typically occurs within hours to days following the original insult (e.g., ischemia or nephrotoxin insult). Thus, the polypeptides described herein can be administered before the insult, or within an hour to 30 days (e.g., 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 15 days, 20 days, 25 days, 28 days, or 30 days) after the insult (e.g., a surgery or nephrotoxin insult described herein).

A subject can be determined to have, or have the risk of developing, acute kidney injury based on, e.g., the Risk Injury Failure Loss ESRD (RIFLE) criteria or the Acute Kidney Injury Network criteria (Bagshaw et al., Nephrol. Dial. Transplant., 23 (5):1569-1574 (2008); Lopes et al., Clin. Kidney J., 6(1):8-14 (2013)).

In certain embodiments, the methods of this disclosure involve determining measuring the levels of one or more of: serum, plasma or urine creatinine or blood urea nitrogen (BUN); measuring the levels of serum or urine neutrophil gelatinase-associated lipocalin (NGAL), serum or urine interleukin-18 (IL-18), serum or urine cystatin C, or urine KIM-1, compared to a healthy control subject, to assess whether the subject has, or has a risk of developing, acute kidney injury.

The efficacy of the polypeptides of the invention can be assessed in various animal models. Animal models for acute kidney injury include those disclosed in e.g., Heyman et al., Contrin. Nephrol., 169:286-296 (2011); Heyman et al., Exp. Opin. Drug Disc., 4(6): 629-641 (2009); Morishita et al., Ren. Fail., 33(10):1013-1018 (2011); Wei Q et al., Am. J. Physiol. Renal Physiol., 303(11):F1487-94 (2012).

The efficacy of treatments may be measured by a number of available diagnostic tools, including physical examination, blood tests, measurements of blood systemic and capillary pressure, proteinuria (e.g., albuminuria), microscopic and macroscopic hematuria, assessing serum creatinine levels, assessment of the glomerular filtration rate, histological evaluation of renal biopsy, urinary albumin creatinine ratio, albumin excretion rate, creatinine clearance rate, 24-hour urinary protein secretion, and renal imaging (e.g., MRI, ultrasound).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Expression of Full Length DKK2 and DKK2 C2 Domain

A series of 11 constructs were engineered to express the full length and C2 only domains of DKK2, consisting of 6 human and 5 murine versions with and without a His-Avi tag. The specifics of the constructs are summarized in the Table 2 below.

TABLE 2
List of Constructs

| Construct | Plasmid | DKK2 | Scrambling | Expression |
|---|---|---|---|---|
| FL-huDKK2- [symbol] | pBKM086 | DKK2 hu | scrambled | multiple faint |
| | | | disulfides? | bands by |
| | | | | western |
| EngSS-huDKK2(S26-I259)- [symbol] | pBKM087 | DKK2 hu | | none by |
| | | | | western |
| EngSS-huDKK2-C2(M172-I259)- [symbol] | pBKM088 | DKK2 hu | | none by |
| | | | | western |
| FL-muDKK2- [symbol] | pBKM092 | DKK2 hu | scrambled | very faint by |
| | | | disulfides | western |
| EngSS-muDKK2(S26-I259)- [symbol] | pBKM093 | DKK2 hu | scrambled | very faint by |
| | | | disulfides | western |
| EngSS-huDKK2-C2(M172-I259)- [symbol] | pBKM094 | DKK2 hu | scrambled | barely noticed |
| | | | disulfides | on western |
| EngSS-huDKK2-C2(M172-I259) | pBKM098 | DKK2 hu | | faint bands |
| | | | | by western |
| EngSS-huDKK2(S26-I259) | pBKM099 | DKK2 hu | | faint bands |
| | | | | by western |
| EngSS-muDKK2-C2(M172-I259) | pBKM100 | DKK2 hu | scrambled | faint bands |
| | | | disulfides | by western |
| EngSS-muDKK2(S26-I259) | pBKM101 | DKK2 hu | scrambled | faint bands |
| | | | disulfides | by western |
| huDKK2- [symbol] | pACE443 | DKK2 hu | | none by |
| | | | | western |

[symbol]

indicates data missing or illegible when filed

As can be seen in this table, expression of molecules from these constructs was not seen or was seen only faintly by western blot. In addition, many of the constructs yielded molecules with disulfide scrambling. Further details are provided below.

a. Expression of Untagged DKK2 in CHO Cells.

Tagless versions of human and mouse full-length DKK2 and DKK2 C2 were expressed transiently in CHO cells. Due to the calculated pI at approximately 9-9.5, cation exchange with SP sepharose and heparin sepharose were tested. Untagged DKK2 FL and DKK2-C2 were unable to be purified from CHO cells using the conditions tested (data not shown). Since SDS-PAGE of culture supernatant and eluted fractions showed no obvious bands for DKK2 or DKK2 C2 when samples were analyzed by SDS-PAGE stained with Coomassie blue, a western blot was developed to track the presence of the protein. Presence of full-length human and mouse DKK2 was negative or barely detectable by western blots. Western blots of human and mouse DKK2 C2 showed the protein present at the expected molecular weight under reduced conditions, but the non-reduced lanes indicated that this material was highly aggregated and that the aggregates were held together by scrambled disulfide cross-links. The C2 domain has 10 Cys residues which form 5 disulfide bonds based on the structures of DKK1 and DKK2 C2 domains. The presence of higher molecular weight bands under non-reducing SDS-PAGE/Western analysis indicates incorrect disulfide formation as there are no free cysteines in the protein.

The amino acid sequence of some of the constructs described above are set forth below.

Human DKK2 C2 (pBKM098)
Full ORF
(SEQ ID NO: 1)
MGFLPKLLLLASFFPAGQAMSHIKGHEGDPCLRSSDCIEGFCCARHFWTKICKPVLHQGE
VCTKQRKKGSHGLEIFQRCDCAKGLSCKVWKDATYSSKARLHVCQKI
Mature polypeptide (signal IP prediction)
(SEQ ID NO: 2)
MSHIKGHEGDPCLRSSDCIEGFCCARHFWTKICKPVLHQGEVCTKQRKKGSHGLEIFQR
CDCAKGLSCKVWKDATYSSKARLHVCQKI
Number of amino acids: 88
Molecular weight: 9979.6
Theoretical pI: 9.11
Human DKK2 Full length (pBKM099)
Full ORF
(SEQ ID NO: 3)
MGFLPKLLLLASFFPAGQASQIGSSRAKLNSIKSSLGGETPGQAANRSAGMYQGLAFGG
SKKGKNLGQAYPCSSDKECEVGRYCHSPHQGSSACMVCRRKKKRCHRDGMCCPSTRC
NNGICIPVTESILTPHIPALDGTRHRDRNHGHYSNHDLGWQNLGRPHTKMSHIKGHEGD
PCLRSSDCIEGFCCARHFWTKICKPVLHQGEVCTKQRKKGSHGLEIFQRCDCAKGLSCK
VWKDATYSSKARLHVCQKI
Mature Polypeptide (signal IP prediction)
(SEQ ID NO: 4)
SQIGSSRAKLNSIKSSLGGETPGQAANRSAGMYQGLAFGGSKKGKNLGQAYPCSSDKEC
EVGRYCHSPHQGSSACMVCRRKKKRCHRDGMCCPSTRCNNGICIPVTESILTPHIPALDG
TRHRDRNHGHYSNHDLGWQNLGRPHTKMSHIKGHEGDPCLRSSDCIEGFCCARHFWTK
ICKPVLHQGEVCTKQRKKGSHGLEIFQRCDCAKGLSCKVWKDATYSSKARLHVCQKI
Number of amino acids: 234
Molecular weight: 25808.5
Theoretical pI: 9.42
Mu DKK2 C2 (pBKM100)
Full ORF
(SEQ ID NO: 5)
MGFLPKLLLLASFFPAGQAMPHIKGHEGDPCLRSSDCIDGFCCARHFWTKICKPVLHQG
EVCTKQRKKGSHGLEIFQRCDCAKGLSCKVWKDATYSSKARLHVCQKI
Mature polypeptide (signal IP prediction)
(SEQ ID NO: 6)
MPHIKGHEGDPCLRSSDCIDGFCCARHFWTKICKPVLHQGEVCTKQRKKGSHGLEIFQR
CDCAKGLSCKVWKDATYSSKARLHVCQKI
Number of amino acids: 88
Molecular weight: 9975.6
Theoretical pI: 9.11
Mu DKK2 Full Length (pBKM101)
Full ORF
(SEQ ID NO:7)
MGFLPKLLLLASFFPAGQASQLGSSRAKLNSIKSSLGGETPAQSANRSAGMNQGLAFGG
SKKGKSLGQAYPCSSDKECEVGRYCHSPHQGSSACMLCRRKKKRCHRDGMCCPGTRC
NNGICIPVTESILTPHIPALDGTRHRDRNHGHYSNHDLGWQNLGRPHSKMPHIKGHEGD
PCLRSSDCIDGFCCARHFWTKICKPVLHQGEVCTKQRKKGSHGLEIFQRCDCAKGLSCK
VWKDATYSSKARLHVCQKI
Mature polypeptide (signal IP prediction)
(SEQ ID NO: 8)
SQLGSSRAKLNSIKSSLGGETPAQSANRSAGMNQGLAFGGSKKGKSLGQAYPCSSDKEC
EVGRYCHSPHQGSSACMLCRRKKKRCHRDGMCCPGTRCNNGICIPVTESILTPHIPALDG
TRHRDRNHGHYSNHDLGWQNLGRPHSKMPHIKGHEGDPCLRSSDCIDGFCCARHFWT
KICKPVLHQGEVCTKQRKKGSHGLEIFQRCDCAKGLSCKVWKDATYSSKARLHVCQKI
Number of amino acids: 234
Molecular weight: 25728.4
Theoretical pI: 9.43

b. Expression of His-Tagged DKK2 in CHO Cells.

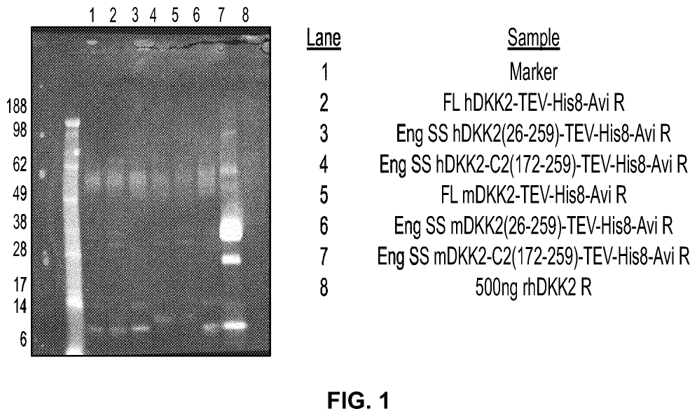

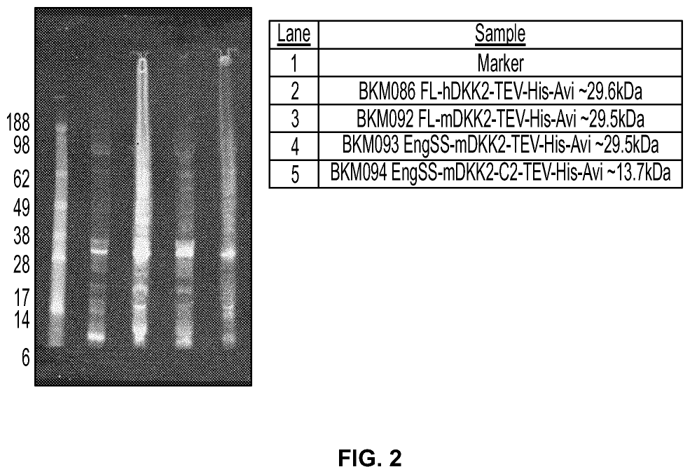

His-tagged DKK2 constructs from stable pools were also tested, showing similar negative or very poor expression results (FIG. 1). No intact DKK2 (molecular weight of approximately 30 kDa for DKK2 C1+C2) was visible on the western analysis of the cell culture supernatants. Western blots of human and mouse DKK2 C2 showed the protein present at the expected molecular weight under reduced conditions, but the non-reduced lanes indicated that this material was highly aggregated and that the aggregates were held together by disulfide cross-links. His-tagged DKK2 FL and DKK2-C2 were unable to be purified from CHO cells using the conditions tested. High salt (1M) cell washes of selected samples were also evaluated and were found to contain DKK2, but the material was highly aggregated as was apparent by diffuse staining of DKK2 with molecular weight of greater than 30 kDa and/or fragmented apparent from the presence of lower molecular weight bands of less than 29.5 kDa for full length DKK2 C1+C2 and less than 13.7 kDa for DKK2 C2 (FIG. 2). Attempts to improve solubility by including dextran sulfate in the growth medium led to slightly higher levels in the conditioned medium but the proteins were fragmented and/or aggregated.

c. Expression of His-Tagged DKK2 in E. coli.

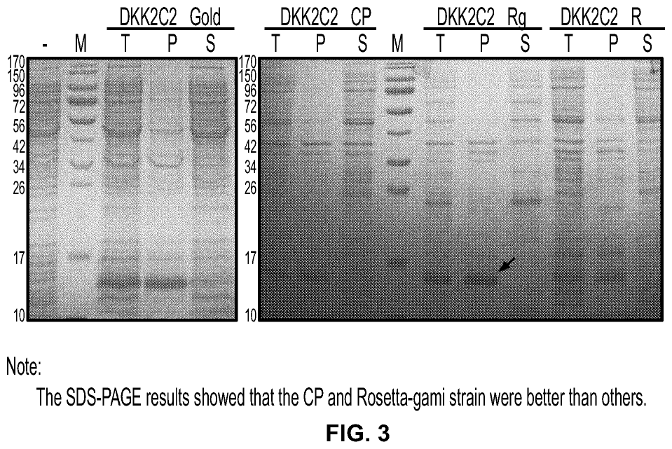

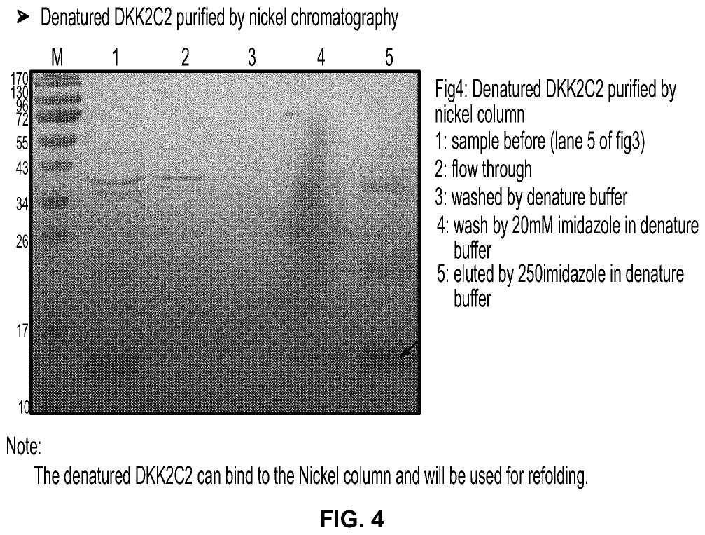

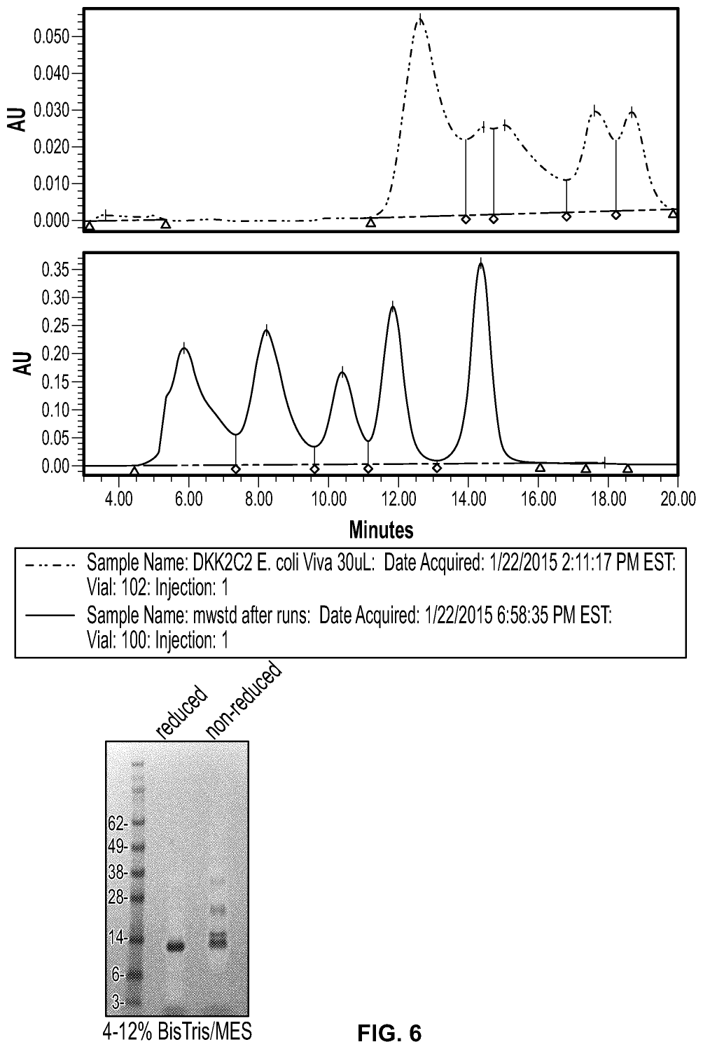

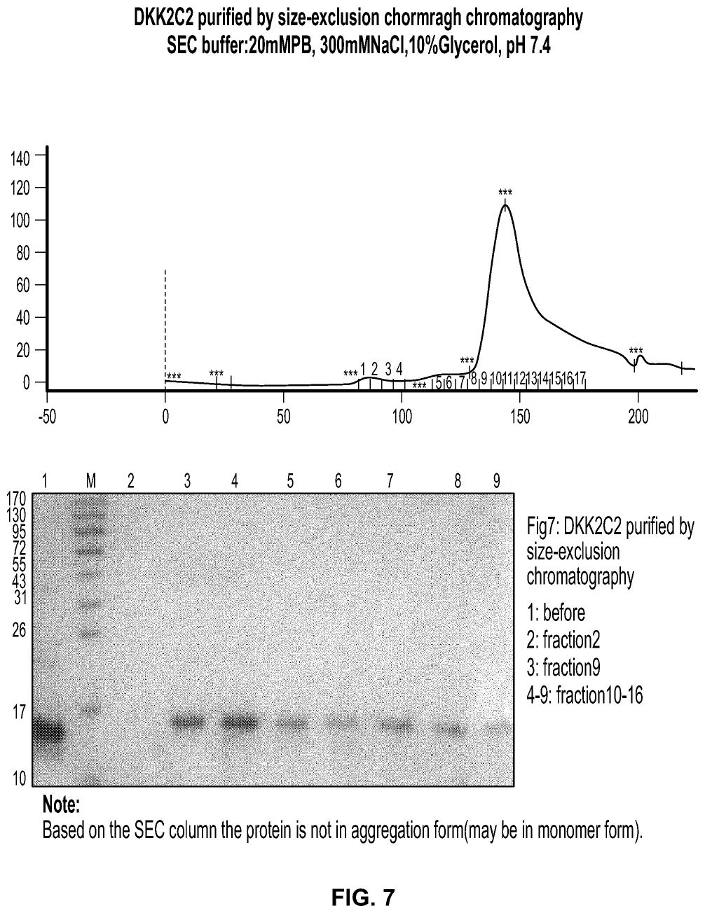

To increase the chances of generating hDKK2-C2 for testing in bioassays, expression of a his tagged version of the protein was tested in E. coli. First, it was determined that this version of hDKK2-C2 goes into inclusion bodies (FIG. 3). The pellet was solubilized with 8M Urea and his DKK2C2 purified using Ni chromatography (FIG. 4). Four different buffer systems were used for testing refolding conditions to generate monomeric hDKK2-C2:
Refolding buffer A: 50 mM Tris, 240 mM NaCl, 10 mM KCl, 1 mM GSSG, 5 mM GSH pH 8.0
Refolding buffer B: 50 mM Tris, 240 mM NaCl, 10 mM KCl, 0.25 M L-Arginine 1 mM GSSG, 5 mM GSH pH 6.0
Refolding buffer C: 20 mM PB, 240 mM NaCl, 10 mM KCl, 1 mM GSSG, 5 mM GSH pH 6.0
Refolding buffer D: 20 mM PB, 240 mM NaCl, 10 mM KCl, 0.25M L-Arginine, 1 mM GSSG, 5 mM GSH pH 6.0
Dialysis buffer: 20 mM PB, 10% glycerol, 300 mM NaCl, pH 7.4,
   FIG. 5 shows the results using refolding buffer C. Analysis of the refolded sample showed heterogeneity by SEC and mixed disulfide mediated aggregation by SDS-PAGE under non-reducing conditions (FIG. 6). Further purification by SEC yielded a monomer form of the protein (FIG. 7).

### Example 2: E. coli Derived DKK-C2 Untagged

A further effort to express DKK2 as a C2 fragment, untagged, was undertaken. The C2 domain of murine DKK2 (DKK2-C2) was produced in E. coli using the methods described in U.S. Pat. No. 8,470,554. The molecular biology and expression were performed as closely as possible to the methods described in the patent. Slight modifications to the purification protocol in the patent were implemented.

a. Construction of Expression Vector

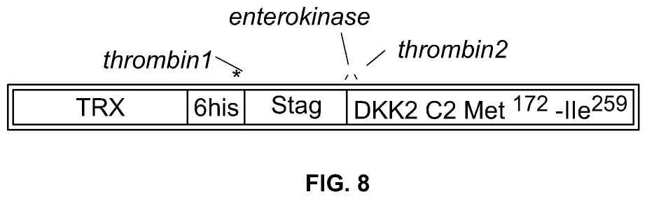

DNA encoding the mouse DKK2-C2 expression cassette was synthesized and cloned into pET32a using 5′ NdeI & 3′ BamHI sites. The synthetic DNA consisted of an N-terminal thioredoxin (TRX), hexa-his (SEQ ID NO:9) tag, thrombin cleavage sequence, s-tag, enterokinase cleavage sequence, a second thrombin cleavage site, and DKKC2 (Met172-Ile259, Genbank NM_020265) (FIG. 8). The DNA sequence was optimized for expression in E. coli. and is provided below.

(SEQ ID NO: 10)
ATGAGCGATAAAATTATTCACCTGACTGACGACAGTTTTGACACGGATGT
ACTCAAAGCGGACGGGGCGATCCTCGTCGATTTCTGGGCAGAGTGGTGCG
GTCCGTGCAAAATGATCGCCCCGATTCTGGATGAAATCGCTGACGAATAT
CAGGGCAAACTGACCGTTGCAAAACTGAACATCGATCAAAACCCTGGCAC
TGCGCCGAAATATGGCATCCGTGGTATCCCGACTCTGCTGCTGTTCAAAA
ACGGTGAAGTGGCGGCAACCAAAGTGGGTGCACTGTCTAAAGGTCAGTTG
AAAGAGTTCCTCGACGCTAACCTGGCCGGTTCTGGTTCTGGCCATATGCA
CCATCATCATCATCATTCTTCTGGTCTGGTGCCACGCGGTTCTGGTATGA
AAGAAACCGCTGCTGCTAAATTCGAACGCCAGCACATGGACAGCCCAGAT
CTGGGTACCGACGACGACGACAAGGCCCTGGTGCCGCGTGGCAGCATGCC
GCACATTAAAGGCCATGAAGGCGATCCGTGCCTGCGTAGCTCTGATTGCA
TTGATGGCTTTTGTTGCGCGCGTCATTTTTGGACCAAAATTTGTAAACCG
GTGCTGCATCAGGGCGAAGTGTGCACCAAACAGCGTAAAAAAGGCAGCCA
TGGGCTGGAGATCTTTCAGCGTTGCGATTGCGCGAAAGGCCTGAGCTGCA
AAGTGTGGAAAGATGCAACCTATAGCAGCAAAGCGCGTCTGCATGTGTGC
CAGAAGATATAATGAGGATCC

The amino acid sequence encoded by the above nucleic acid sequence is provided below (TRX boldened; hexa-his (SEQ ID NO:9) tag underlined; s-tag italicized; thrombin sites boldened and underlined; enterokinase site italicized and underlined; and DKK2-C2 in lower case):

(SEQ ID NO: 11)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTDDDDKALVPRGSmphikghegdpclrssdcidgfccarhfwtkickp
vlhqgevctkqrkkgshgleifqrcdcakglsckvwkdatysskarlhvc
qki

b. Expression

The Trx-Dkk2-C2 fusion expression vector was transformed into an ORIGAMI™ B strain of E. coli (Invitrogen) for protein production. Cells were grown in Luria-Bertani media with shaking at 220 rpm at 37° C. Protein expression was induced by the addition of 0.2 mM isopropyl-1-thio-3-D-galactoside (IPTG) when cells were at about mid-log phase (OD_{600 nm} approximately 0.5) and the culture was shifted to 16° C. after IPTG addition and incubated for an additional 16 hours. SDS-PAGE (sodium dodecyl sulfate polyacrylamide gel electrophoresis) was used to verify protein expression.

c. Purification

Following 16 hour induction, cells were harvested by centrifugation at 5000 Kg for 20 minutes at 4° C. Cell pellet was resuspended in lysis buffer (25 mM Bis-Tris, pH 6.8, 500 mM NaCl, 5 mM MgCl₂, and 2% Glycerol) containing protease inhibitor cocktail (Roche Diagnostics, Germany) at a ratio of 1 volume lysis volume:25 volumes original culture volume. Cell suspension was passed through a Microfluidizer Processor (Model M110L, Microfluidics, Newton, Mass.) twice at 10,000 psi. The cell lysate was centrifuged at 13,000 rpm for 20 minutes at 4° C. to remove the insoluble fraction.

The clarified lysate was purified on a NiNTA agarose (Qiagen) column using 1 nil resin/10 ml lysate using gravity flow. The column was equilibrated with 5 column volumes in lysis buffer (25 mM Bis-Tris, pH 6.8, 500 mM NaCl, 5 mM. MgCl₂, and 2% Glycerol) and the lysate was passed over the column twice. The column was washed with 5 column volumes lysis buffer followed by 5 column volumes of lysis buffer containing 50 mM imidizole. The fusion protein was eluted with 5 column volumes lysis buffer containing 250 mM imidizole.

After IMAC purification, the target protein DKK2-C2 was cleaved from the Trx-Dkk2-C2 fusion by removing the thioredoxin-tag, His₆ (SEQ ID NO:9)-tag and S-tag regions with thrombin. The NiNTA purified fusion protein was incubated with thrombin from human plasma (Sigma) at a ratio of 100 units/l of original culture for 4 hours at room temperature and then overnight at 4 degrees. Benzamidine-sepharose 4 FE (GE Healthcare) was added to the cleavage reaction to bind thrombin (2 ml/1000 units) and terminate digestion. Benzamidine-sepharose resin was removed by passing the mixture over a disposable column. U.S. Pat. No. 8,470,554 reported that with even more extensive thrombin treatment than this, only the first N-terminal thrombin site would be cleaved, leaving a S-tagged DKK2 C2. However, in these experiments it was observed that both thrombin sites were cleaved (mass spectrometry). It was not possible to control the thrombin digest adequately to obtain the S-tagged protein. Mass Spectrometry analysis of a sample from the lab issuing the patent also did not have the N-terminal S-tag.

After thrombin cleavage, the DKK2-C2 protein was separated from the proteolytic fragments using reverse phase chromatography. Acetic acid was added to a final concentration of 5%. A heavy precipitant formed and was removed by centrifugation at 5000×g for 20 minutes and the supernatant was filtered. SDS-PAGE confirmed that the DKK2-C2 was retained in the supernatant and suggested the precipitant was non-protein. DKK2-C2 protein was loaded onto C8 SepPak column (Waters) equilibrated in 0.1% TFA. A 5 g column was used for a 10-liter prep. The column was washed with 5 column volumes 0.1% TFA, then 5 column volumes each of 0.1% TFA with 10%, 20%, 30%, and 40% acetonitrile. Samples were lyophilized and dissolved in PBS and analyzed by mass spectrometry. The 20% acetonitrile elution contained mostly the desired DKK2-C2 (164-253 of the fusion protein). Elution fractions from 30% and 40% acetonitrile contained primarily the N-terminal fragments with the thioredoxin, His, and S-tag. Non-reduced mass spectrometry of reduced and non-reduced samples showed the 20% acetonitrile fraction contained 5 disulfide bonds, however further disulfide analysis identified significant scrambling. Formulation was changed from a neutral pH in PBS to PBS at pH 6 to decrease disulfide scrambling. Despite significant disulfide scrambling, the protein was active in the Wnt signaling Super Top Flash (STF) activity assay with an IC50 of approximately 20 nM. Pharmacokinetics analysis in mice revealed rapid clearance from serum.

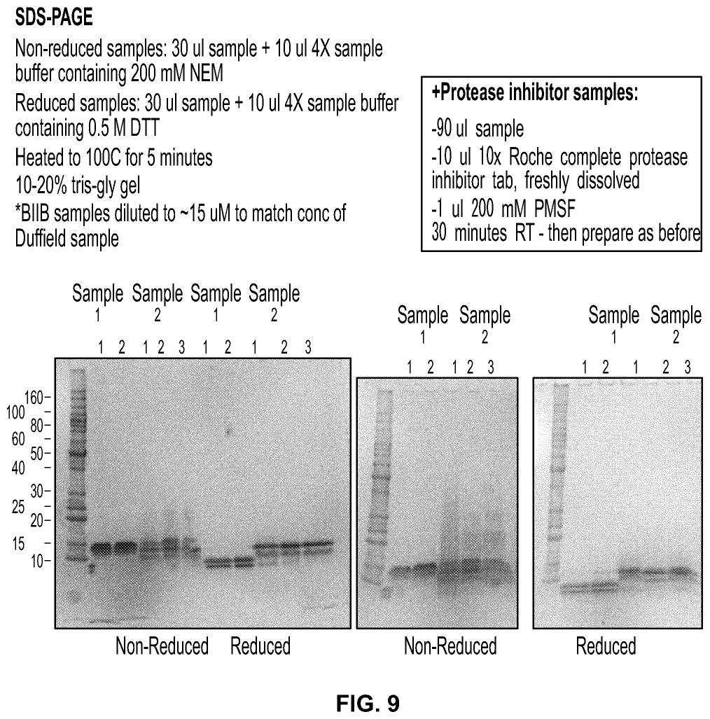

Two independent preparations of DKK2-C2 (Sample 1 and Sample 2) showed multiple bands on reducing SDS-PAGE due to proteolysis (FIG. 9). Sample 2 shows multiple bands under non-reducing conditions consistent with the disulfide scrambling seen by mass spectrometry.

In order to generate a high titer polyclonal antibody targeting DKK2 C2 needed for analysis of pharmacokinetic samples, rabbits were immunized with DKK2 C2 material from E. coli (described in Example 2). Rabbits were treated with a primary and then two secondary boosts (0.5 mg/rabbit) two weeks apart. On days 24 and 28 after the primary injection, production bleeds were taken and serum was prepared. ELISA titers on the immunogen were 1:>40,000 dilution. The DKK2 specific antibody was affinity purified by loading rabbit anti-sera onto a Neutravidin agarose (ThermoScientific) column preloaded with biotinylated E. coli DKK2 C2 material@ 0.25 mg per ml resin. To generate a biotinylated version, 1.5 mg/ml E. coli DKK2 C2 in 25 mM HEPES pH7.5 was incubated with EZ-Link NHS-PEG4-Biotin (ThermoScientific) to 0.3 mM final concentration at room temperature for 1 hour. The reaction was stopped with ethanolamine and pH adjusted with 0.5M MES pH6 buffer. To generate the DKK2 affinity column, the biotinylated E. coli DKK2 C2 material was diluted 150-fold in PBS pH 7.4 and bound in batch to Neutravidin agarose at room temperature for 1 hour with column end-over-end mixing. The resin was washed three times with 8-bed volumes of PBS and then 3-bed volumes of anti-serum were loaded in a column format. Following six single bed volume washes with PBS, bound antibody was eluted in four fractions (each one column volume) with 25 mM sodium acetate pH3.2, 100 mM NaCl, and antibody-containing fractions were neutralized with HEPES pH7. Affinity-purified antibody was biotinylated by incubating with a 20-fold molar excess of EZ-Link NHS-PEG4-biotin for 30 minutes at room temperature. The reaction was stopped with the addition of ethanolamine and pH adjustment with 0.5M MES pH6 buffer and desalted to remove unreacted biotin on a Zeba spin desalting column (Thermo Scientific).

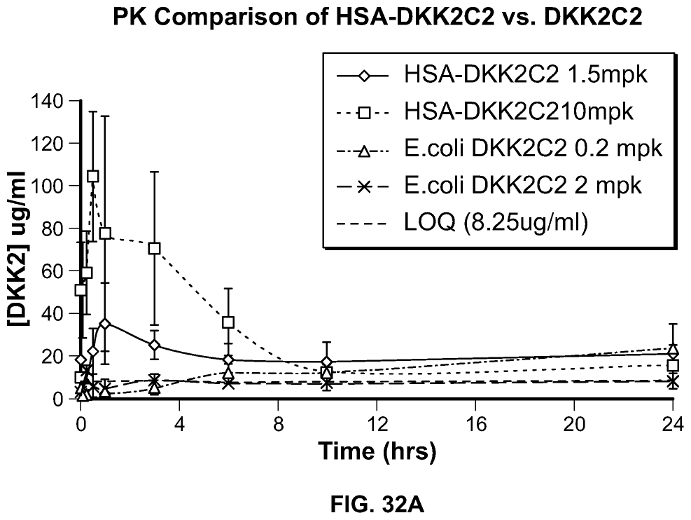

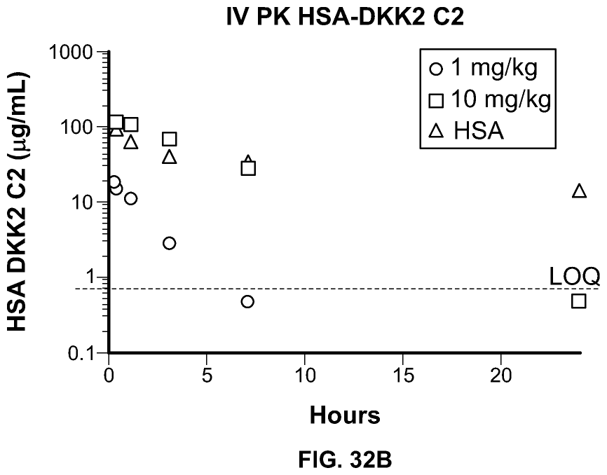

Mice (3/group) were injected intravenously with 2 mg/kg DKK2 C2 material from E. coli. Blood was drawn and serum prepared after 5 min, 15 min, 30 min, 1 hr, 3 hrs, 6 hrs, 10 hrs and 24 hrs. Levels of DKK2 C2 in the serum were measured using an ELISA protocol against a standard curve of DKK2 C2 in mouse serum. Specifically, serum samples were diluted 1:10 in PBS and coated onto a Nunc clear flat-bottom immuno non-sterile 96-well plate (ThermoFisher Scientific) blocked earlier with fish gelatin blocking buffer (PBS, 0.5% fish gelatin, 0.1% Triton X-100 pH 7.4). A standard curve of E. coli DKK2 C2 spiked into 10% mouse serum/PBS in a concentration series starting at 2 ug/ml, preceded by seven 3-fold dilutions in 10% mouse serum/PBS, was included on the same plate. Following three washes with PBST, wells were incubated at room temperature for 1 hour with the biotinylated version of the affinity-purified anti-DKK2 C2 antibody at 2 ug/ml in blocking buffer. Following three washes with PBST, wells were incubated at room temperature for 15 minutes with streptavidin-HRP (ThermoFisher Scientific) in a 1:8000 dilution in blocking buffer. Following three washes with PBST, wells were incubated at room temperature for 4 minutes with TMB substrate (0.1M NaAc citric acid pH4.9, 0.42 mM TMB, 0.004% hydrogen peroxide). Developed ELISAs were stopped by the addition of 2N sulfuric acid and plates were scanned at 450 nm using a Molecular Devices SpectraMax M5 microplate reader and data analyzed using Softmax Pro v5.4.4 software. For the 5 min time point samples DKK2 C2 was detected in serum at a level of between 10-30 ng/ml, while for all other serum samples detection was below the limit of quantitation of 1 ng/ml. These results are consistent with the low levels measured in the STF assay using functional activity of DKK2 as a readout, where again levels in serum were below the limit of quantitation (FIG. 32A).

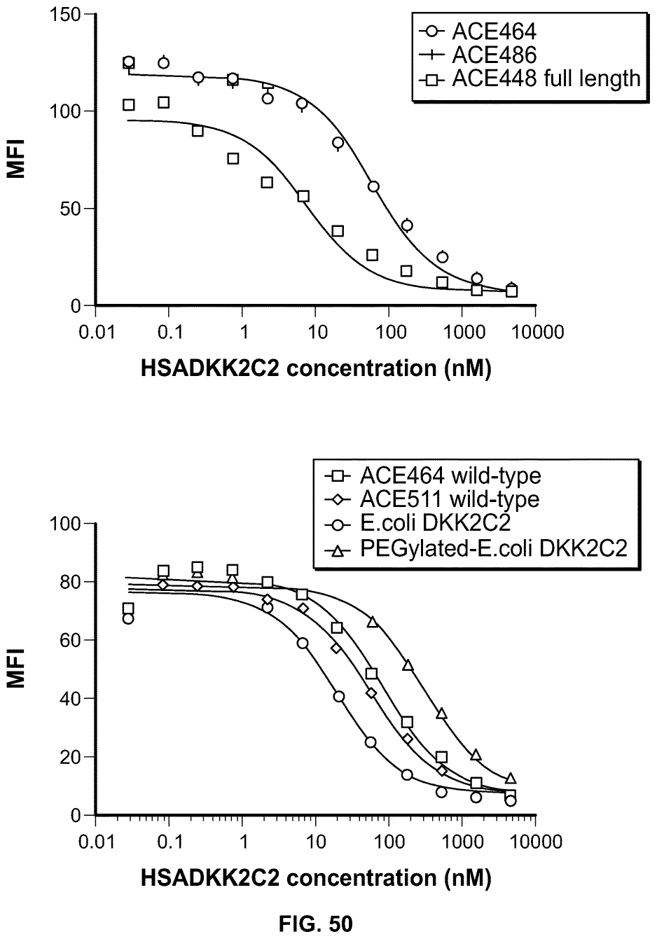

To generate a PEGylated version of DKK2 C2 from E. coli (described in Example 2) to test for LRP6 binding, 1.5 mg of DKK2 C2 in PBS pH6 was incubated with 15 mg of 20k-PEG-2-methyl proprionaldehyde (BioVectra) and sodium cyanoborohydride to 20 mM, at room temperature in the dark for overnight. Greater than 90% of the DKK2 C2 was monoPEGylated. Monomeric PEGylated DKK2 C2 was purified from dimeric and non-PEGylated forms by size exclusion chromatography on a Superdex200 10/300 column (GE Healthcare) at a flow rate of 0.5 ml/min in PBS pH6. Ten microliters from each 0.5 ml fraction was loaded onto a 4-12% Bis-Tris NuPAGE gel in MES buffer under non-reducing conditions. The gel was run at 200V for 35 minutes and stained with SimplyBlue SafeStain (ThermoFisher Scientific). Fractions containing monoPEGylated DKK2 C2 were pooled and concentrated for LRP6 binding assays. PEGylation of untagged DKK2 C2 from E. coli increased the IC50 of LRP6 binding from 20 nM to 326 nM (FIG. 50) as measured by FACS.

### Example 3: Purification of Fc-hDKK2-C2

Fc fusions were next made in an attempt to improve the characteristics of DKK2 molecules.

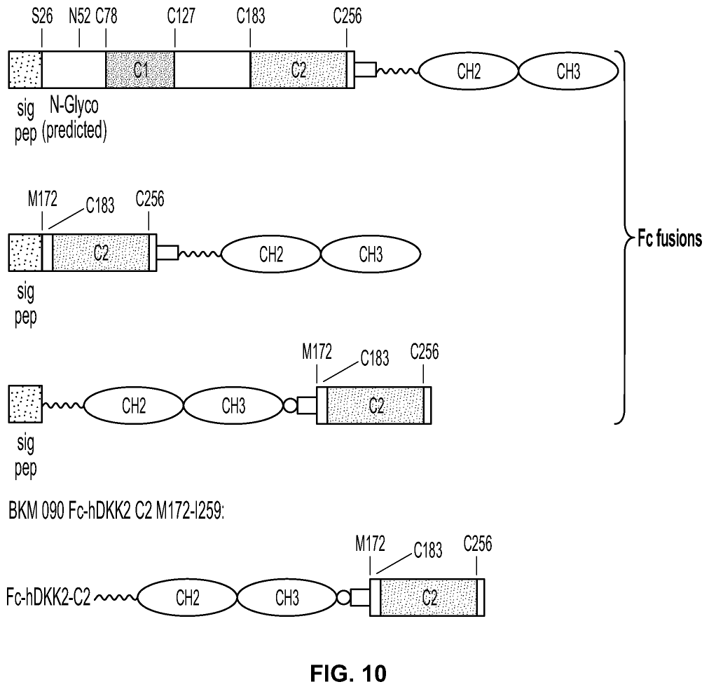

A series of 6 constructs were evaluated as part of the analysis of fusions of DKK2 to Fc, 3 using human DKK2 sequences (BKM 091 hDKK2 C1+C2 S26-I259-Fc, BKM 089 hDKK2 C2 M172-I259-Fc, and BKM 090 Fc-hDKK2 C2 M172-I259) and 3 using the same construct design but with murine DKK2 sequences (BKM 097 mDKK2 C1+C2 S26-I259-Fc, BKM 095 mDKK2 C2 M172-I259-Fc, and BKM 096 Fc-mDKK2 C2 M172-I259). The schematic in FIG. 10 summarizes the various designs.

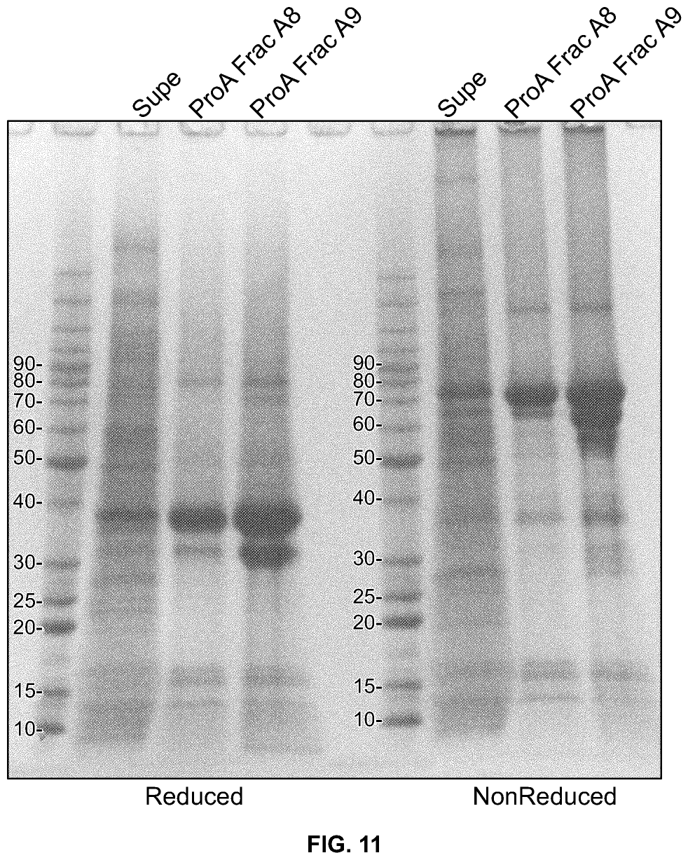

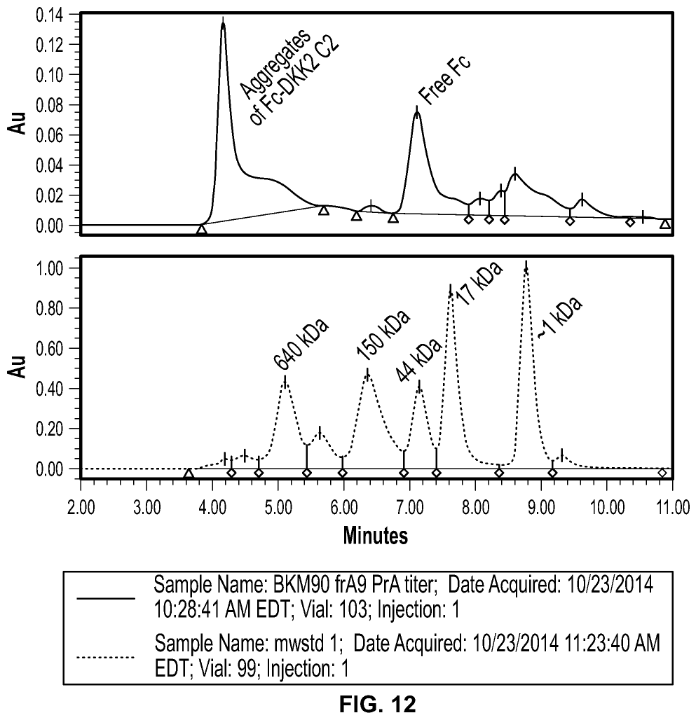

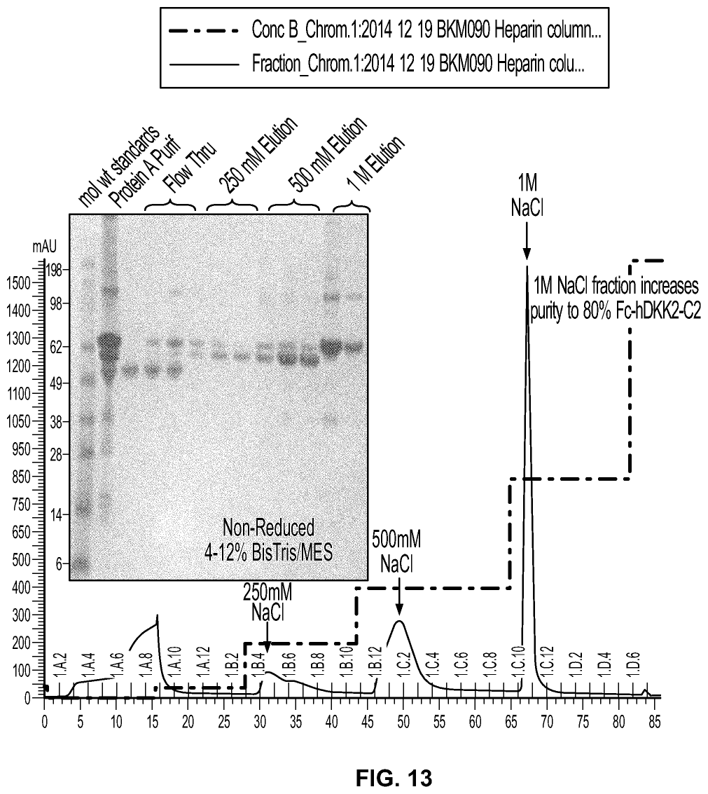

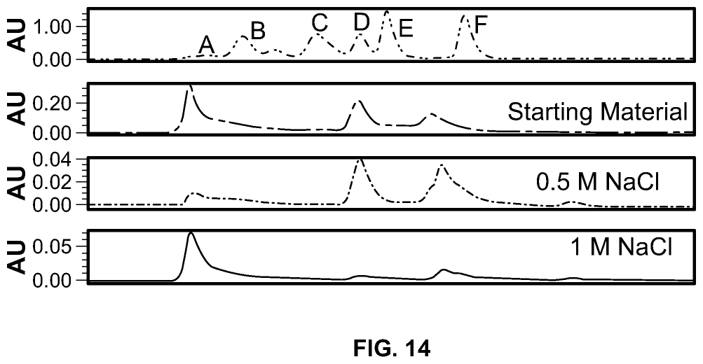

Fc-fusions of DKK2 were purified on Protein A and ion exchange chromatography, but for all constructs extensive clipping occurred between DKK2 and Fc fusion (FIG. 11). Mass spectrometry results of the 4 DKK2-C2 alone containing Fc-constructs showed intact protein to be the following: 55% for Fc-hDKK2-C2, 27% for hDKK2-C2-Fc, 5% for mDKK2-C2-Fc, and 3% for Fc-mDKK2-C2. Of these versions BKM 090 Fc-hDKK2 C2 M1724259 looked most promising when samples were characterized by SDS-PAGE, but the preparation was highly aggregated and in the void volume of the column when characterized by size exclusion chromatography (FIG. 12). Intact Fc-hDKK2 C2 in the protein A eluate was further purified by cation exchange chromatography where it eluted later than the clipped forms of the protein (FIG. 13). The 1M salt fraction contained Fc-hDKK2 C2 with about 80% running at a molecular weight of 70 kDa and a series of discrete higher molecular weight disulfide linked aggregates of the protein and a lower molecular weight fragment. An analysis of cation exchange elution fractions from FIG. 13 by analytical size exclusion chromatography showed that over 80% of the protein in the 1M salt fraction from the cation exchange column eluted in the void volume of the column MW greater than 640 kDa, indicating that it is highly aggregated and no visible peak was observed at expected molecular weight for the fusion protein of 70 kDa (FIG. 14). SEC running buffers containing 1 M NaCl, 2 M MgCl₂, 1 M Arginine, or 0.5 M Urea were also tested and shown to impact the elution time of the Fc-hDKK2-C2, with 1 M NaCl and 2 M MgCl₂ having an elution time in the region between 44-150 kDa; however, dynamic light scattering (DLS) of the samples still showed high polydispersity and a large aggregate.

Characterization of the purified BKM090 protein on a LCT Premier mass spectrometer following purification on Protein A and PNGase treatment revealed that about 50% had the expected mass and 50% contained cleavages at 10 different positions in the sequence. Following purification on Heparin Sepharose the purity reached 80% and 8 cleavage fragments were detected, but following dialysis to remove the high salt additional cleavage occurred. Table 3 shows mass spectrometry data generated for the sample before and after dialysis.

### BKM090: Fc-G4S-TEV-huDKK2-C2(M172-I259)

Sequence with Predicted Signal Sequence Shown in Lower Case and Italics:

(SEQ ID NO: 91)
metdtlllwvlllwvpgstgDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGGGGS
ENLYFQSMSHIKGHEGDPCLRSSDCIEGFCCARHFWTKICKPVLHQGEVC
TKQRKKGSHGLEIFQRCDCAKGLSCKVWKDATYSSKARLHVCQKI

TABLE 3
Mass spectrometry results for the reduced, deglycosylated
BKM090 ProtA (201412161), BKM090 Hep1M (201412162),
and BKM090 dialyzed (201412163)

| Detected Mass (Da) | | | | Predicted |
|---|---|---|---|---|
| ProtA | HepIM | dialyzed | Possible Assignment | Mass (Da) |
| | 9762 | | BKM090, res. 260-345 | 9,761.5 |
| 9849 | 9850 | 9850 | BKM090, res. 259-345 | 9,848.5 |
| 10068 | 10068 | 10068 | BKM090, res. 257-345 | 10,066.8 |
| | | 25807 | BKM090, res. 21-251 | 25,808.2 |
| 25922 | | | BKM090, res. 21-252 | 25,922.3 |
| 26199 | | | BKM090, res. 21-254 | 26,198.6 |
| 26474 | 26474 | 26474 | BKM090, res. 21-254 | 26,473.9 |
| 26561 | | | BKM090, res. 21-254 | 26,561.0 |
| 26693 | 26693 | 26692 | BKM090, res. 21-254 | 26,692.2 |
| 31917 | | | BKM090, res. 21-254 | 31,916.3 |
| | | 32173 | BKM090, res. 21-254 | 32,172.6 |
| | | 32754 | BKM090, res. 21-254 | 32,753.2 |
| 35347 | 35348 | | BKM090, res. 21-254 | 35,345.3 |
| 36479 | 36479 | 36480 | unknown | |
| 36524 | 36524 | 36524 | BKM090, res. 21-345 | 36,522.7 |
| (~51%) | (~80%) | (~46%) | | |

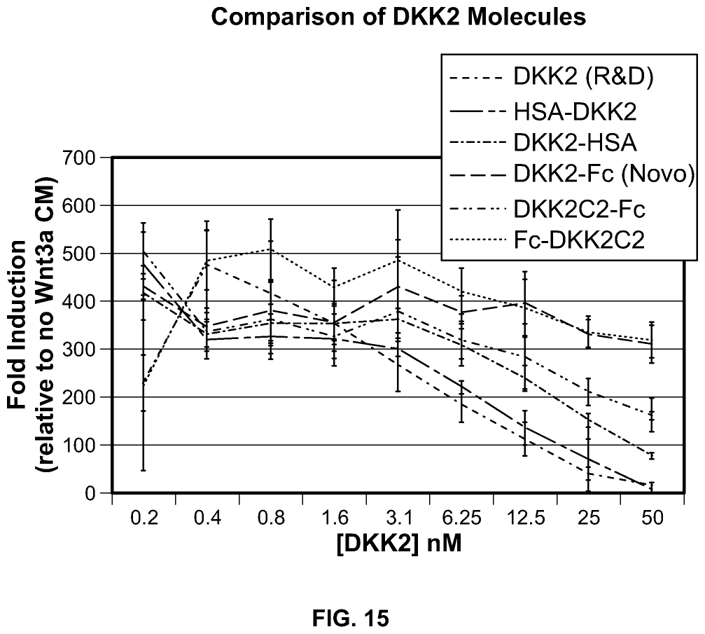

Analysis of the Fc fusions in the Super Top Flash Assay (FIG. 15) found that the activity of Fc-DKK2-C2 was lower than expected for other DKK2 C2 proteins (not tested in this analysis).

The table below lists conditions that were evaluated in an attempt to improve the quality of the Fc-DKK2 C2 protein during expression. In addition to evaluation in CHO cells, the construct was also expressed in 293 cells.

| Proteins | Additive |
|---|---|
| Fc-hDKK2-C2 | Dextran Sulfate |
| | FBS |
| Fc-DKK2-C2 + hLRP6-His | Dextran Sulfate |
| Fc-DKK2-C2 + Kremen2-His | Dextran Sulfate |

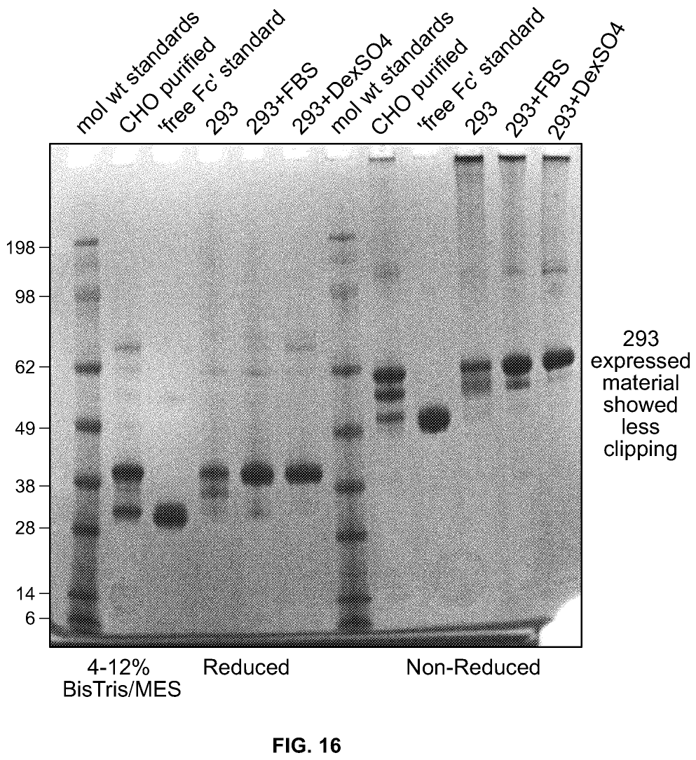

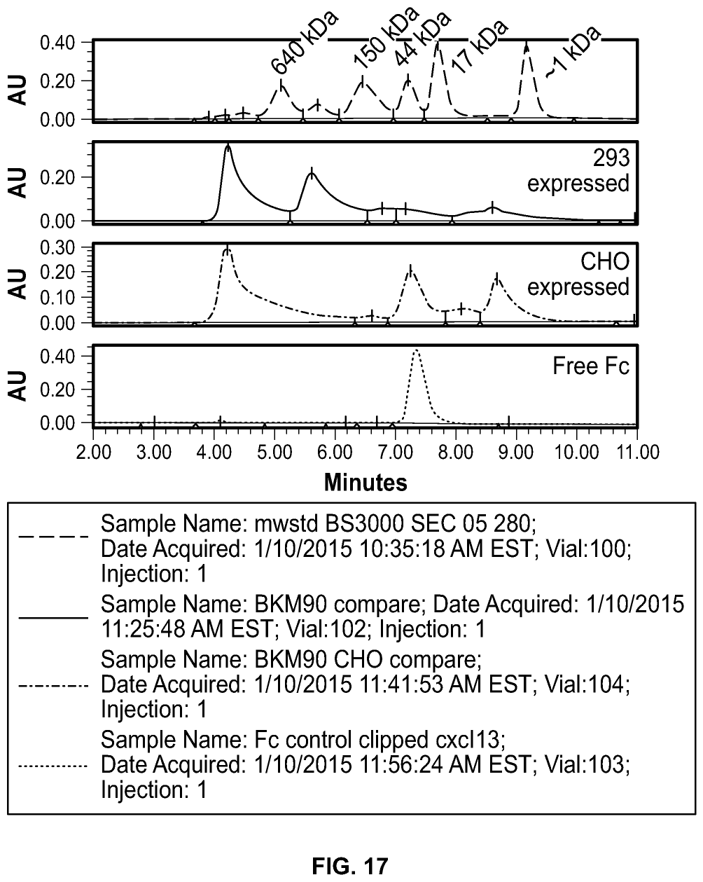

As seen in CHO cells, there was significant clipping of the protein in 293 cells and a larger fraction of the protein formed high molecular weight aggregates seen under non-reducing conditions. The amount of clipping was somewhat improved when the cells were cultured in the presence of fetal bovine serum and significantly improved when the cells were cultured in the presence of dextran sulfate (FIG. 16). When 293 samples were characterized by SEC (FIG. 17), they were also found to be highly aggregated.

Co-expression studies with protein partners Kremen-2 or LRP6 were also tested, but were inconclusive since these proteins did not appear to be expressed in similar amounts as the DKK2 Fe fusions.

Constructs BKM 091 hDKK2 C1+C2 S26-1259-Fc, BKM 089 hDKK2 C2. M172-1259-Fc were also expressed in CHO cells and purified by protein A chromatography. SDS-PAGE analysis showed that both protein preparations contained numerous clipped forms. No protein band representing intact full length DKK2-Fc (BKM 091) was observed with only a protein band migrating at the size of a free-Fe present in the Protein A eluate. For the DKK2-C2-Fc fusions, there were protein bands present that migrated at the expected molecular weights in both non-reduced and reduced samples; although there was more clipping in the DKK2-C2-Fc sample than with Fc-DKK2-C2. Analytical SEC indicated that purified DKK2-C2-Fc, like what had been observed with Fc-DKK2-C2, was highly aggregated. Dextran Sulfate in the conditioned media was also tested and showed an improved titer by Octet; however, protein A purified material showed similar clipped forms and were also aggregated by analytical SEC. Mass spectrometry analysis of DKK2-C2 Fe revealed that only 27% of the purified protein was intact and that 73% contained cleavages at 10 sites (see Table 4 below).

BKM089: huDKK2-C2(M172-I259)-TEV-Fc
Sequence with Predicted Signal Sequence Shown in Lower Case and Italics:

(SEQ ID NO: 92)
mgflpkllllasffpagqamSHIKGHEGDPCLRSSDCIEGFCCARHFWTKI
CKPVLHQGEVCTKQRKKGSHGLEIFQRCDCAKGLSCKVWKDATYSSKARLH
VCQKIENLYFQSKTHTCPPCPAPELLGGPSVFLFPPKPKDTLMISRTPEVT
CVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVD
KSRWQQGNVFSCSVMHEALHNHYTQKSLSLSPG

TABLE 4

| Detected | | Predicted | |
|---|---|---|---|
| Mass (Da) | Possible Assignment | Mass (Da) | Notes |
| 23529 | unknown | | |
| 23816 | BKM089, res. 129-339 | 23817.0 | ~73% |
| 24171 | BKM089, res. 126-339 | 24,172.4 | |
| 24837 | BKM089, res. 119-339 | 24,838.2 | |
| 25177 | BKM089, res. 116-339 | 25,177.6 | |
| 25392 | BKM089, res. 114-339 | 25,392.8 | |
| 25668 | BKM089, res. 112-339 | 25,668.1 | |
| 25831 | BKM089, res. 111-339 | 25,831.3 | |
| 27009 | BKM089, res. 101-339 | 27,009.7 | |
| 27236 | BKM089, res. 99-339 | 27,237.0 | |
| 27451 | BKM089, res. 97-339 | 27,451.3 | |
| 36018 | BKM089, res. 21-339 | 36,018.2 | ~27% |

### Example 4: Expression and Purification of XTEN-DKK2

XTEN was also used as a fusion partner to the DKK2 proteins. Table 5 below summarizes the constructs and the expression levels.

TABLE 5

| Construct | Plasmid | DKK2 | Expression | |
|---|---|---|---|---|
| XTEN144-hDKK2 | pACE476 | DKK2 | western and | multiple |
| C2 (H174-I259) | | hu | coomassie | bands, very |
| | | | positive | hetero- |
| | | | | geneous |
| XTEN144-hDKK2 | pACE475 | DKK2 | very faint | Q column |
| (S26-I259) | | hu | bands on | |
| | | | western | |

The amino acid sequence of the XTEN construct (SEQ ID NO:12) in pACE475 is provided below:

ACE475: XTEN144-hDKK2 (S26-I259)
Sequence:
SPAGSPTSTEEGTSESATPESGPGTSTEPSEGSAPGSPAGSPTSTEEGTST
EPSEGSAPGTSTEPSEGSAPGTSESATPESGPGSEPATSGSETPGSEPATS
GSETPGSPAGSPTSTEEGTSESATPESGPGTSTEPSEGSAPGSQIGSSRAK
LNSIKSSLGGETPGQAANRSAGMYQGLAFGGSKKGKNLGQAYPCSSDKECE
VGRYCHSPHQGSSACMVCRRKKKRCHRDGMCCPSTRCNNGICIPVTESILT
PHIPALDGTRHRDRNHGHYSNHDLGWQNLGRPHTKMSHIKGHEGDPCLRSS
DCIEGFCCARHFWTKICKPVLHQGEVCTKQRKKGSHGLEIFQRCDCAKGLS
CKVWKDATYSSKARLHVCQKI

| | |
|---|---|
| Number of amino acids: 378 | 33.9% XTEN144 |
| Molecular weight: 39021.6 | 66.1% hDKK1 |
| Theoretical pI: 6.56 | |

The amino acid sequence of the XTEN construct (SEQ ID NO:13) in pACE476 is provided below:

ACE476: XTEN144-hDKK2 C2 (H174-I259)
Sequence:
SPAGSPTSTEEGTSESATPESGPGTSTEPSEGSAPGSPAGSPTSTEEGTST
EPSEGSAPGTSTEPSEGSAPGTSESATPESGPGSEPATSGSETPGSEPATS
GSETPGSPAGSPTSTEEGTSESATPESGPGTSTEPSEGSAPGHIKGHEGDP
CLRSSDCIEGFCCARHFWTKICKPVLHQGEVCTKQRKKGSHGLEIFQRCDC
AKGLSCKVWKDATYSSKARLHVCQKI

| | |
|---|---|
| Number of amino acids: 230 | 57.6% XTEN144 |
| Molecular weight: 22974.5 | 42.4% hDKK1 |
| Theoretical pI: 4.73 | |

a. Purification of ACE 476 on a Q-Sepharose Column.

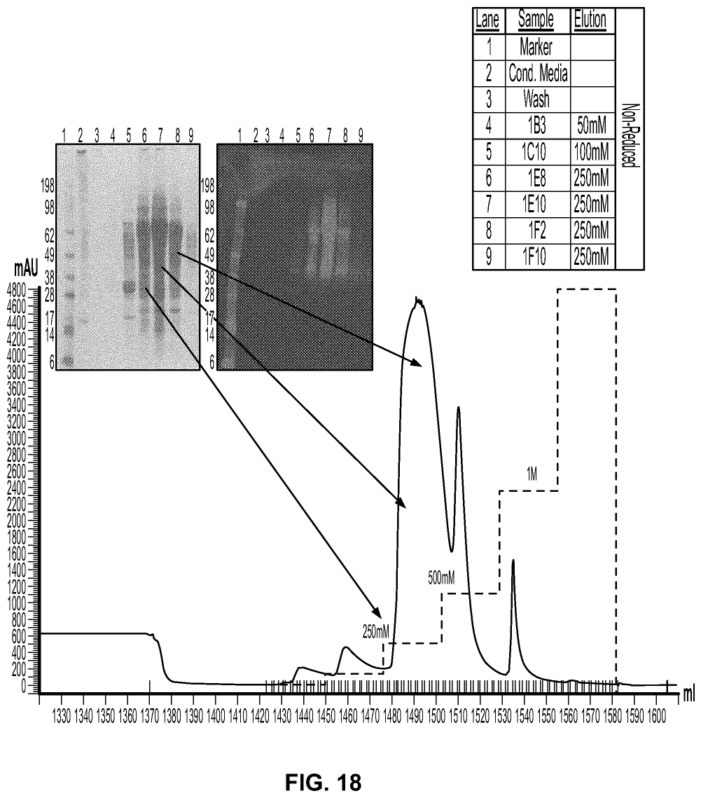

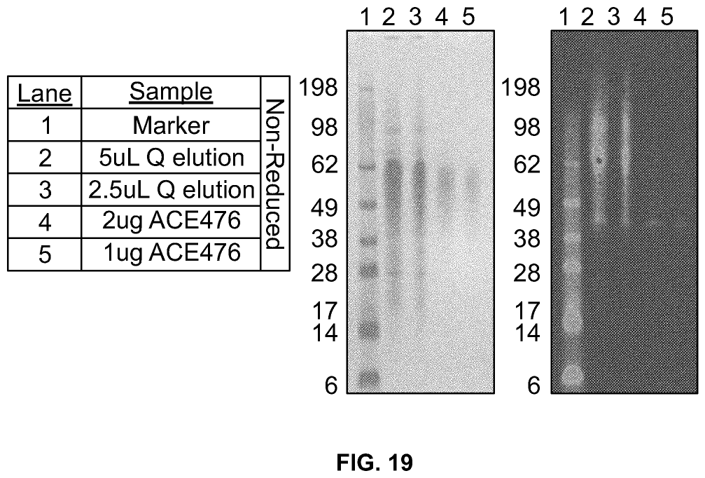

ACE476, XTEN144-hDKK2 C2 (H174-I259), was identified from column fractions using anti-DKK2 antibody raised against a peptide that recognized the C-terminus of the DKK2-C2 domain. Immunoreactive components ranged in molecular weight from approximately 40-100 kDa indicating that the expressed protein was very heterogeneous (FIG. 18) and contained disulfide scrambling that led to formation of the higher molecular weight forms. No further separation occurred when the sample was further fractionated on phenyl Sepharose and reloaded and eluted from a second Q-Sepharose column (FIG. 19).

b. Purification of ACE 475 on a Q-Sepharose Column.

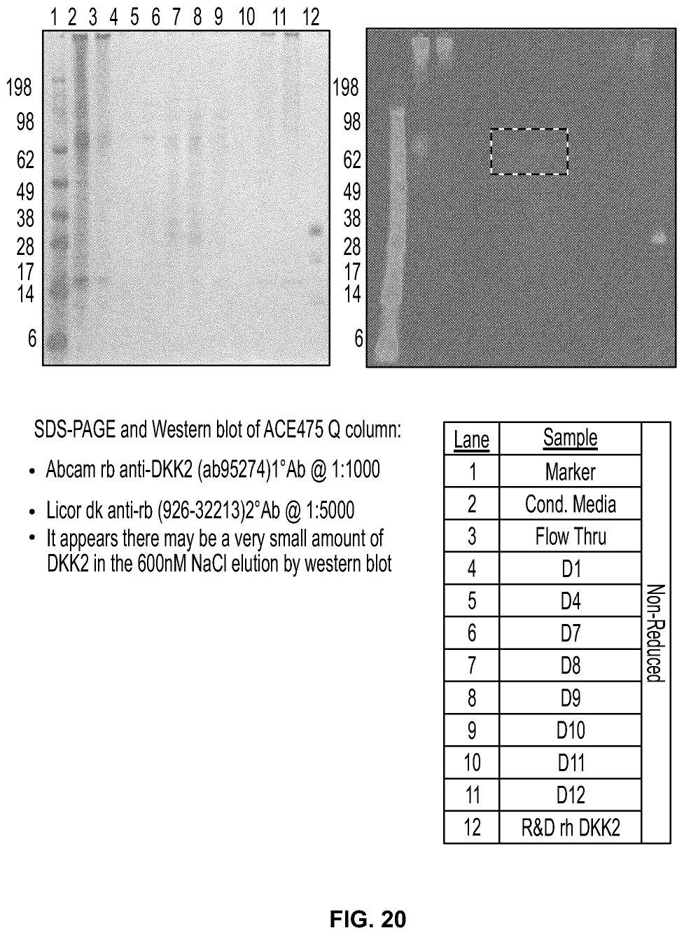

When ACE475 XTEN144-hDKK2 (S26-I259) was loaded onto Q-Sepharose only a fraction of the protein bound as evident by the presence of immunoreactive product in the flow through fraction from the column (FIG. 20). The column eluate (fractions D7 and D8) contained a broad immunoreactive band with molecular weight ranging from 65-75 kDa. From the commassie stained profile this material represents at best only about 10-20% of the total protein in the sample. A significant amount of the immune reactive protein was in high molecular weight aggregates that barely entered the SDS-PAGE indicating significant disulfide scrambling (present in load, flow through, and later eluting column fractions).

### Example 5: Purification and Characterization of HSA Fusions of Full Length DKK2

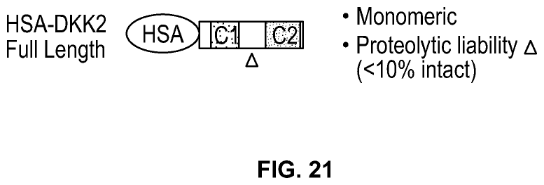

Full length DKK2 molecules were fused to human serum albumin (HSA) in an attempt improve expression and post expression attributes of the molecule. Several constructs of full length DKK2 were contemplated. The first of these made was ACE 448 HSA-DKK2 full length (C1+C2) (FIG. 21). The protein was purified on CaptureSelect™ HSA and analyzed by SDS-PAGE/Western analysis.

Using CaptureSelect™ for purification, <<5% of the product contained the C-terminal DKK2 peptide in the first purified sample-lane 2 and about 5% in the second preparation-lane 3 (arrowhead in FIG. 21). None of the other bands in the preparation were recognized by the DKK2 antibody.

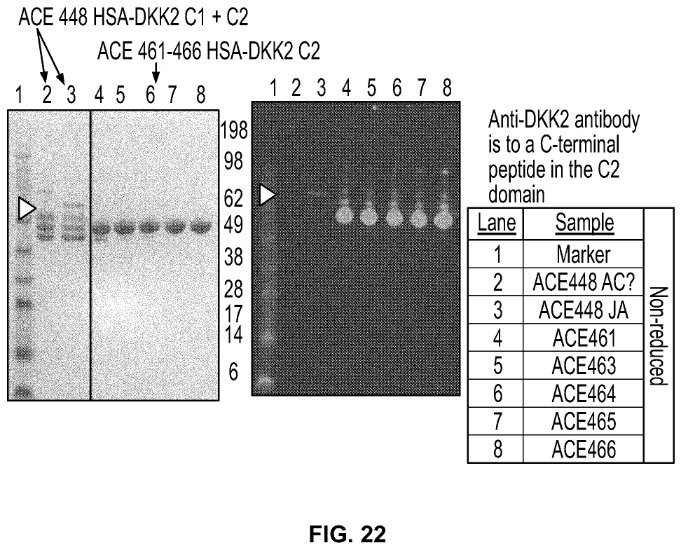

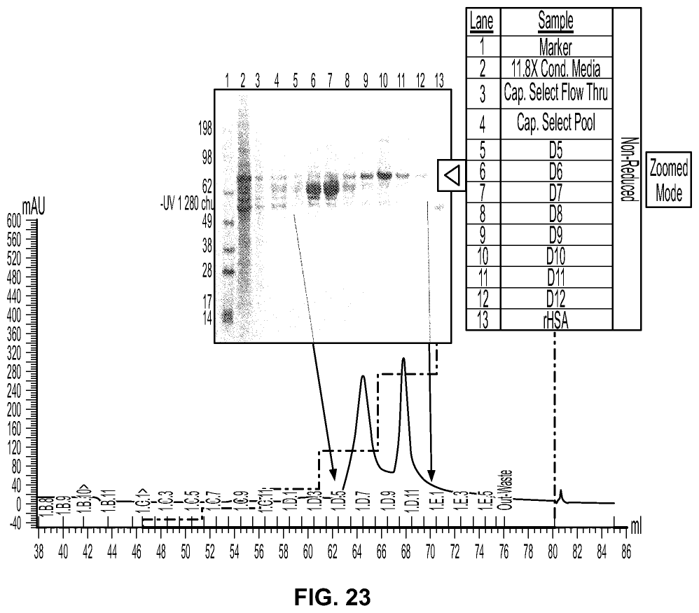

The ACE 448 HSA-DKK2 C1+C2 protein was purified on Heparin Sepharose and column fractions were analyzed by SDS-PAGE (FIG. 22). Fractionation of the conditioned medium on Heparin Sepharose allowed for separation of the full length protein from the other HSA-DKK2 fragments (FIG. 23). At this stage the material was only about 50% pure and required further purification for detailed characterization. After SEC we recovered <10% of the HSA DKK2 that was present in the conditioned medium as intact based on antibody recognition. The material could not be characterized by mass spec+/−PNGase treatment because of heterogeneity in the signal, often a sign of O-linked sugars.

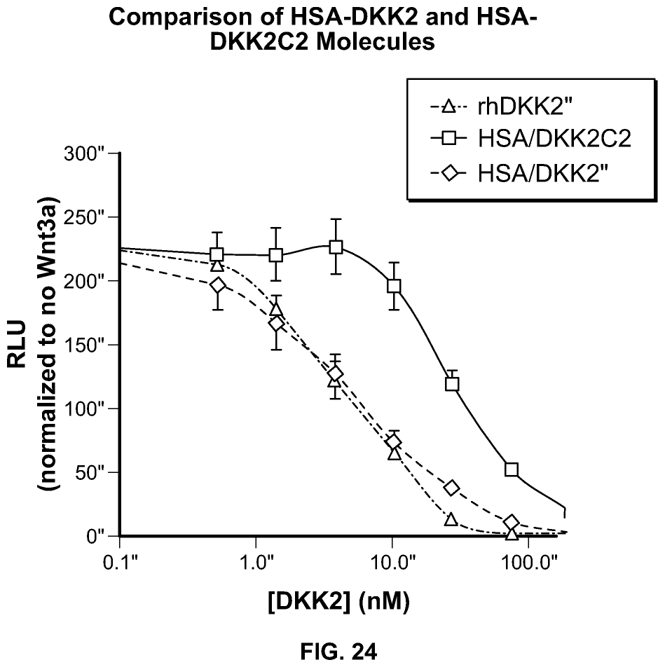

The activity of the purified HSA-DKK2 full length was equivalent to the DKK2 standard without HSA attached (FIG. 24). Consistent with the known contribution of the DKK2 C1 domain to activity, HSA-DKK2 full length was 10 times as active as the HSA-DKK2 C2 proteins. In the context of the full length DKK2 (C1+C2), the C1 domain binds to the first propellar repeat domain of LRP6 and C2 binds to the third propellar repeat domain of LRP6. Simultaneous binding at both sites leads to a geometric increase in affinity. The lower potency of the DKK2 C2 domain reflects the absence of DKK2 C1 binding.

Short term storage of the purified full length ACE 448 HSA-DKK2 C C2 protein at 4° C. lead to clipping of protein and no intact protein remained in the preparation when the sample was analyzed by SDS-PAGE after only 2 weeks at 4° C.

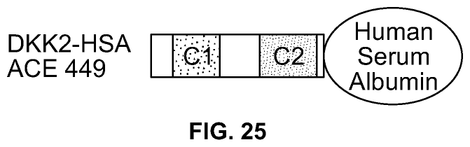

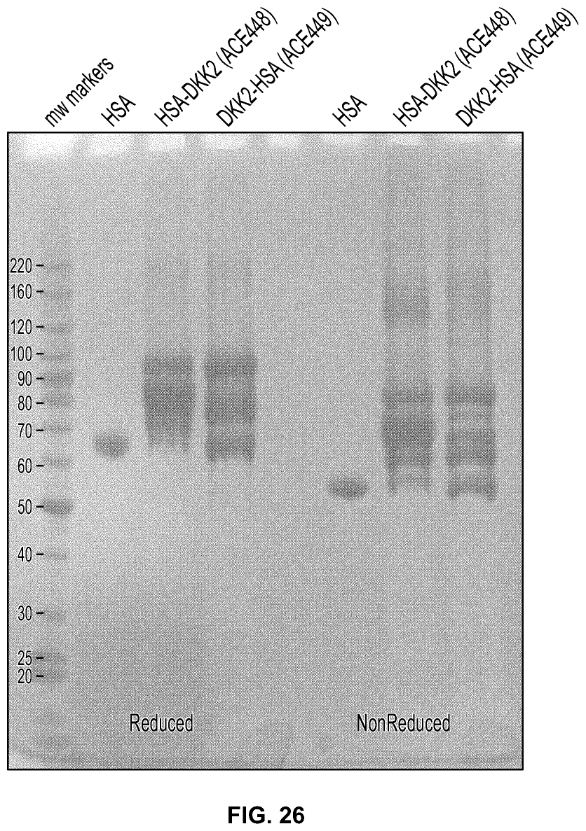

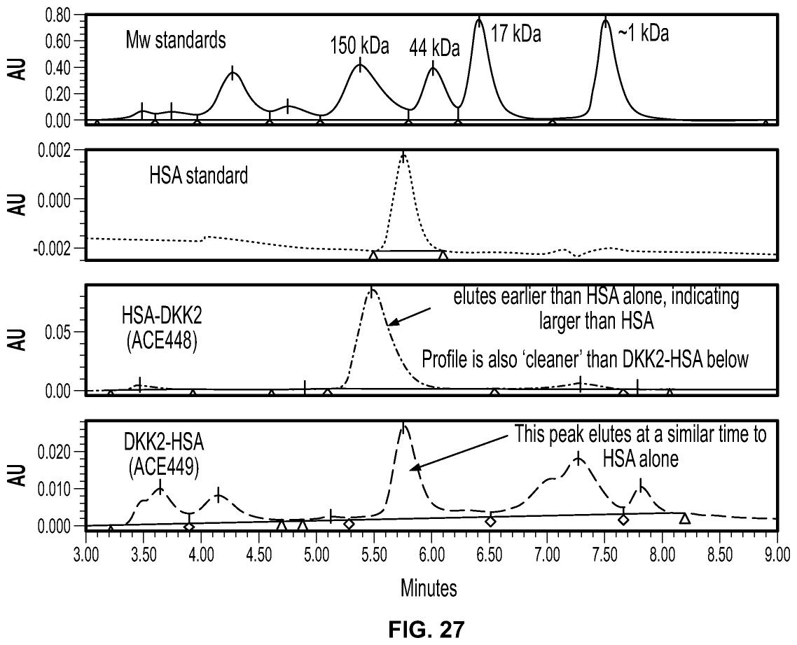

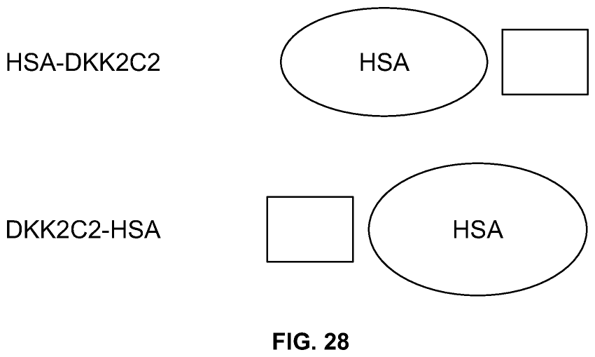

The second DKK2 construct to be studied was ACE 449 DKK2 full length (C1+C2)-HSA (FIG. 25). The protein was purified on CaptureSelect™ HSA and analyzed by SDS-PAGE/Western analysis (FIG. 26). Like ACE 448, CaptureSelect™ HSA purified ACE 449 showed extensive clipping, with bands running from molecular weight of 55 kDa under non-reducing conditions (corresponding to the molecular weight of free HSA) to 80 kDa (corresponding to the molecular weight of the predicted full length product). Analysis of the purified samples by SEC (FIG. 27) revealed that purified ACE 448 eluted from the column as a single peak with the predicted molecular weight of the fusion protein (approximately 80 kDa) whereas the corresponding sample from the ACE 449 purification was very heterogeneous with high molecular weight aggregates eluting early in the chromatogram as well as smaller component visible later in the chromatogram. The predicted molecular weight of the major eluting form is smaller than expected for the full length fusion protein. Activity measurements in the super Top Flash assay revealed that ACE 448 was about 30% more active than ACE 449 (FIG. 15).

### Example 6: Expression of HSA Fusions of the DKK2 C2 Domain

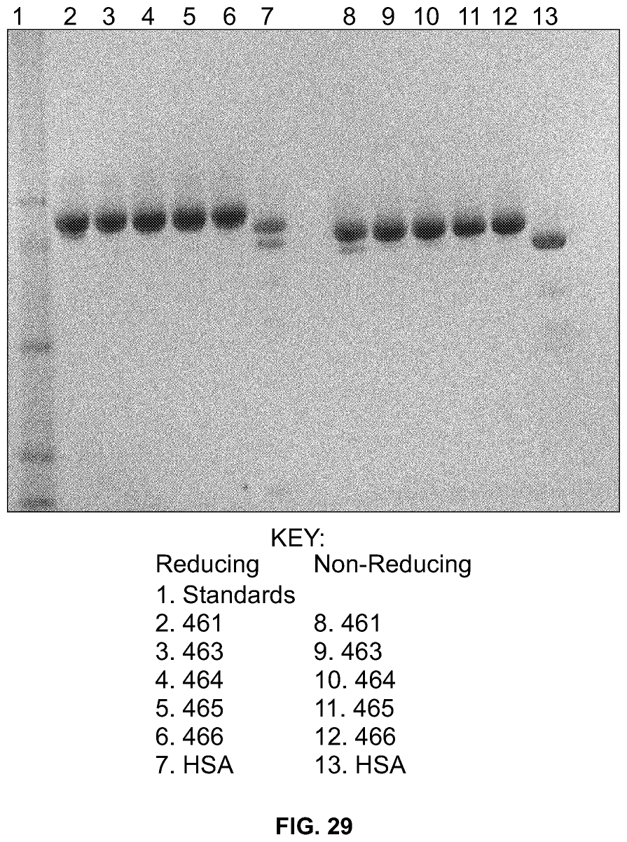

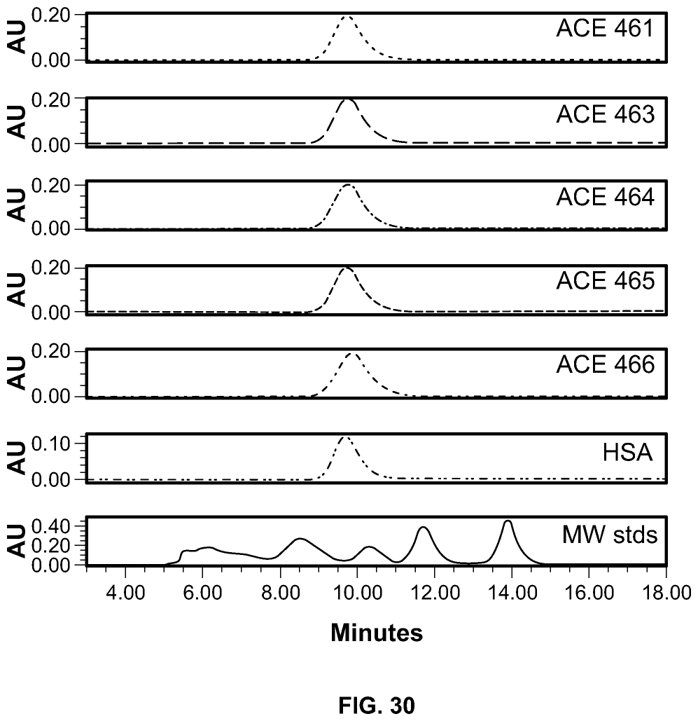

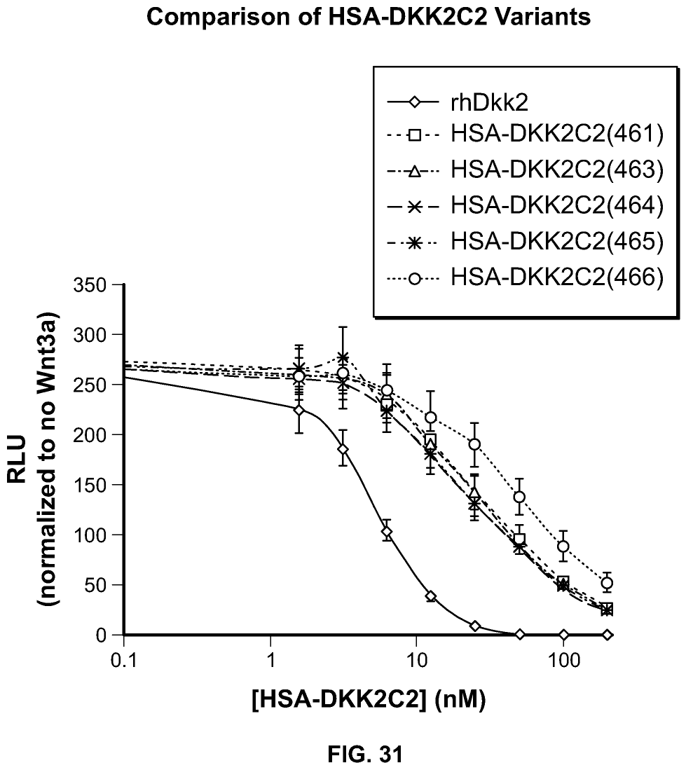

HSA-DKK2 C2 from five constructs (ACE 461: HSA-huDKK2 (M172-I259); ACE 463: HSA-huDKK2 (M172-I259 S173P); ACE 464: HSA-huDKK2 (H174-I259); ACE 465: HSA-huDKK2 (1(176-1259); and ACE 466: HSA-huDKK2 (H178-I259)) were purified from 300 ml of transient culture. For preparation of the conditioned medium transfected CHO cells were expanded in serum-free media, grown to high density, fed with supplements, and shifted to a reduced temperature. Cultures were held at this reduced temperature for up to 14 days or until cell viability started to drop and then harvested by centrifugation and clarified through 0.45 micron filtration. Pilot work was done to demonstrate binding of the fusion proteins to CaptureSelect™ HSA and elution with various buffers at neutral pH (containing 2 M MgCl₂/1M NaCl, 0.5 M arginine/1 M NaCl or 50% propylene glycol/1 M NaCl.) The arginine elution buffer was used for subsequent studies. The CaptureSelect™ HSA affinity purification step was followed by gel filtration on Superdex 200 to remove aggregate and the purified protein was buffer exchanged into 10 mM sodium succinate pH 5.5, 75 mM NaCl, 100 mM arginine. The resulting protein ran as a single band by SDS-PAGE with molecular mass of approximately 70 kDa (FIG. 29), was free of aggregate by analytical SEC (FIG. 30), gave the expected results by mass spec (30% of the protein started at position 21 containing part of the albumin propeptide), and had less than 0.5 EU/mg. All of the proteins were active in the Super Top Flash assay (FIG. 31). ACE 461, 463, 464, 465 were equally active, but ACE 466 was significantly less active.

ACE464 (HSA-hu DKK2 C2 H174-1259) was chosen to scale up production for more detailed studies. ACE464 from 5 L, culture medium from CHO cells following stable transfection of the ACE464 gene was purified on SP Sepharose and size exclusion chromatography on Sephacryl S200. The protein ran as a single band by SDS-PAGE, was free of aggregates by analytical SEC, and was pyrogen free. Mass spectrometry results showed the expected mass with 30% of the protein containing a portion, 7 amino acids, of the HSA pro-domain (calculated mass of intact HSA-hu DKK2 C2 H17442:59 protein, 76360.1 Da; observed mass, 76360 Da: calculated mass of +7 amino acid version of HSA-hu DKK2 C2 H1744259, 77219.1 Da; observed mass, 77221 Da). This larger scale preparation of HSA-DKK2 C2 was used in rat and mouse pharmacokinetics with IV dosing. From the 51, culture about 400 mg of HSA-DKK2 C2 was recovered (greater than 95% pure by SDS-PAGE, <0.25% aggregates by analytical SEC, <0.14 EU/mg protein). The ACE464 (HSA-hu DKK2 C2 H174-1259) protein was very stable with no evidence of degradation after storage for >4 months at 4° C., incubation for 3 days at 37° C., or after multiple freeze-thaw cycles. The disulfide connectivity in the DKK2 region of HSA-DKK2C2 464 was determined by mass spectrometry under reducing and non-reducing conditions following proteolytic digestion of the protein and was as expected with low-level scrambling (FIG. 34 shows disulfide pairing of the 10 cysteines in DKK2 C2 deduced from the published DKK2 C2 NMR structure: Cys1-Cys4, Cys2-Cys5, Cys3-Cys7, Cys6-Cys9, Cys8-Cys10). Also as expected, in the HSA region of the fusion protein, Cys61 is greater than 90% cysteinylated (approximately 8% free) and the major disulfides are as predicted.

To test if the HSA fusion strategy extended pharmacokinetics versus untagged DKK2C2, HSA-DKK2C2 (ACE464) and DKK2C2 were IV injected into mice. Mice were dosed with 1.5 mpk HSA-DKK2C2, 10 mpk of HSA-DKK2C2, 0.2 mpk DKK2C2, or 2 mpk DKK2C2. The differences in doses of HSA-DKK2C2 vs. DKK2C2 account for the difference in molecular weight attributable to the HSA fusion strategy and allows for an equimolar comparison of the two molecules. Serum was tested in the STF assay to determine DKK2C2 molecule concentration as assessed by Wnt inhibitory activity. The HSA fusion strategy (ACE464) greatly extended pharmacokinetics (especially when dosing at 10 mpk), as untagged DKK2C2 cannot be detected above the LOQ of the assay at any time point (FIG. 32A).

HSA-DKK2C2 ACE 464 in rats (and mice) was not detectable after 7 h following 1 mg/kg IV dose or 24 h following a 10 mg/kg IV dose (FIG. 32B). From analysis of the samples by SDS-PAGE with western blotting detected with both anti-HSA and anti-DKK2 C-terminal peptide antibodies, there was no evidence for breakdown of the protein in serum. The short serum half-life is almost certainly due to binding attributes of DKK2C2, since HSA alone has a half-life in rodents of several days. Pharmacokinetics samples were also evaluated using the Super Top Flash assay to measure functional HSA-DKK2C2. HSA-DKK2C2 serum levels measured in the bioassay were indistinguishable from those detected from the Western blot analysis indicating that the administered protein retained activity.

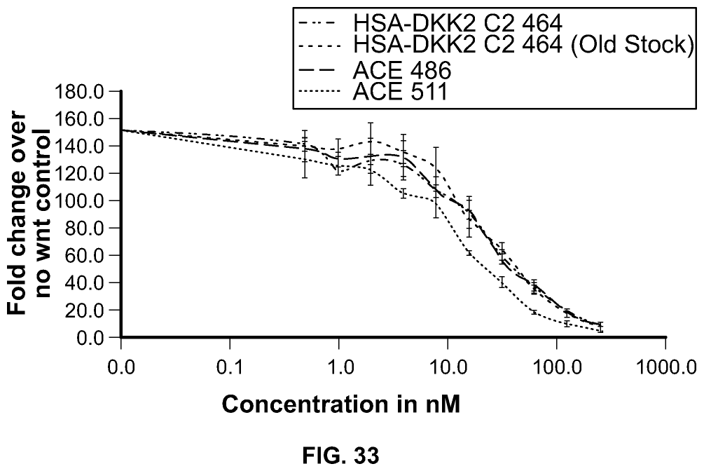

A reengineered version of ACE 464, ACE 486: (HSA-hu DKK2 D25-L609 C2 H174-1259), was also produced to eliminate the heterogeneity of the product caused by the prodomain in the HSA. ACE486 from 600 mL clarified culture medium from CHO cells following stable transfection of the ACE486 gene was purified on SP Sepharose and size exclusion chromatography on Sephacryl 5200. The culture medium as is without dilution or pH adjustment was loaded by gravity onto a 10 mL column (1.5×5.7 cm) SP-Sepharose Fast Flow (GE Healthcare). The column was washed with 2×5 mL of 20 mM sodium phosphate pH 7.0, 50 mM NaCl; 1×5 mL 20 mM sodium phosphate pH 7.0, 100 mM NaCl; and 3×5 mL 20 mM sodium phosphate pH 7.0, 150 mM NaCl. HSA-DKK2C2 was eluted from the column with 20 mM sodium phosphate pH 7.0, 300 mM NaCl, collecting 5×5 mL fractions. Fractions were analyzed for absorbance at 280 nm and by SDS-PAGE. The peak fractions were pooled (20 mL, ˜150 mg), filtered through a 0.2 μm membrane, and concentrated to 12 mL. The protein was loaded onto a 300 mL HiPrep 26/60 Sephacryl 5200 high resolution column (GE Healthcare) in a running buffer of 10 mM sodium succinate pH 5.5, 75 mM NaCl, 100 mM arginine. Samples in the effluent were analyzed for absorbance at 280 nm and by SDS-PAGE. Peak fractions were pooled, filtered, aliquoted, and stored at −70° C. The purified ACE 486 HSA-hu DKK2 D25-L609 C2 H174-I259 protein ran as a single band by SDS-PAGE, was free of aggregates by analytical SEC, and was pyrogen free. Mass spec results showed the expected mass (calculated mass, 76360.1 Da; observed mass, 76363 Da) and the protein was active in the Super Top Flash assay (FIG. 33). ACE486 and ACE464 were also identical in their binding affinities for LRP6 (FIG. 50). The ACE486 framework HSA-hu DKK2 D25-L609 C2 H174-I259 was incorporated in the engineering design of all of the heparin binding mutants.

A construct in which DKK2-C2 was fused at the N-terminus, ACE 462: huDKK2 (NI 72-I259)-HSA, was also produced and characterized. ACE 462 was purified from 300 ml of transient culture on CaptureSelect HSA. The protein had significant proteolysis when analyzed for product quality by SDS-PAGE. Mass spectrometry revealed that the protein was cleaved at the junction of the DKK2 and HSA and was likely due to the presence the HSA prodomain sequence in the construct. The prosequence sequence was subsequently eliminated by reengineering of the construct. ACE 511: huDKK2 (M172-1259)-GS-HSA (D25-L609). ACE511 from 1.5 L culture medium from CHO cells following stable transfection of the ACE511 gene showed no proteolysis at the DKK2-HSA junction. The protein was purified on SP Sepharose and size exclusion chromatography on Sephacryl 5200. The protein ran as a single band by SDS-PAGE, was free of aggregates by analytical SEC, and was pyrogen free. Mass spec results showed the expected mass and the protein was active in the Super Top Flash assay (FIG. 33). In LRP6 binding assays, ACES 11 had an IC₅₀ of 60 nM (FIG. 50), comparable to ACE464 and ACE486.

### Example 7: Amino Acid and DNA Sequences for ACE 461, 463, 464, 465, 466, and 486

The amino acid and nucleic acid sequences of the above-noted HSA-WT DKK2C2 fusions are presented below.

a. pACE461 HSA-A3-huDKK2 C2 (M172-I259):
Amino Acid Sequence including signal peptide (underlined)
(SEQ ID NO: 14)
METDTLLLWVLLLWVPGAHASRGVFRRDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLAAAMSHIKGHEGDPCLRSSDCIEGFCCAR
HFWTKICKPVLHQGEVCTKQRKKGSHGLEIFQRCDCAKGLSCKVWKDATYSSKARLHV
CQKI
DNA sequence including DNA encoding signal peptide (underlined)
(SEQ ID NO: 15)
ATGGAGACAGACACACTCCTGCTATGGGTACTGCTGCTCTGGGTTCCAGGTGCTCAC
GCTTCCAGGGGTGTGTTTCGTCGAGATGCACACAAGAGTGAGGTTGCTCATCGGTTT
AAAGATTTGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAACTGAATTT
GCAAAAACATGTGTTGCTGATGAGTCAGCTGAAAATTGTGACAAATCACTTCATACC
CTTTTTGGAGACAAATTATGCACAGTTGCAACTCTTCGTGAAACCTATGGTGAAATG
GCTGACTGCTGTGCAAAACAAGAACCTGAGAGAAATGAATGCTTCTTGCAACACAA
AGATGACAACCCAAACCTCCCCCGATTGGTGAGACCAGAGGTTGATGTGATGTGCA
CTGCTTTTCATGACAATGAAGAGACATTTTTGAAAAAATACTTATATGAAATTGCCA
GAAGACATCCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGCCAAAGCT
CGATGAACTTCGGGATGAAGGGAAGGCTTCGTCTGCCAAACAGAGACTCAAGTGTG
CCAGTCTCCAAAAATTTGGAGAAAGAGCTTTCAAAGCATGGGCAGTAGCTCGCCTG
AGCCAGAGATTTCCCAAAGCTGAGTTTGCAGAAGTTTCCAAGTTAGTGACAGATCTT
ACCAAAGTCCACACGGAATGCTGCCATGGAGATCTGCTTGAATGTGCTGATGACAG
GGCGGACCTTGCCAAGTATATCTGTGAAAATCAAGATTCGATCTCCAGTAAACTGAA
GGAATGCTGTGAAAAACCTCTGTTGGAAAAATCCCACTGCATTGCCGAAGTGGAAA
ATGATGAGATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGA
CATATGAAACCACTCTAGAGAAGTGCTGTGCCGCTGCAGATCCTCATGAATGCTATG
CCAAAGTGTTCGATGAATTTAAACCTCTTGTGGAAGAGCCTCAGAATTTAATCAAAC
AAAATTGTGAGCTTTTTGAGCAGCTTGGAGAGTACAAATTCCAGAATGCGCTATTAG
TTCGTTACACCAAGAAAGTACCCCAAGTGTCAACTCCAACTCTTGTAGAGGTCTCAA
GAAACCTAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTTGCATGAG
AAAACGCCAGTAAGTGACAGAGTCACCAAATGCTGCACAGAATCCTTGGTGAACAG
GCGACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAGACA
AATCAAGAAACAAACTGCACTTGTTGAGCTTGTGAAACACAAGCCCAAGGCAACAA
AAGAGCAACTGAAAGCTGTTATGGATGATTTCGCAGCTTTTGTAGAGAAGTGCTGCA
AGGCTGACGATAAGGAGACCTGCTTTGCCGAGGAGGGTAAAAAACTTGTTGCTGCA
AGTCAAGCTGCCTTAGGCTTAGCTGCCGCAATGTCACATATAAAAGGGCATGAAGG
AGACCCCTGCCTACGATCATCAGACTGCATTGAAGGGTTTTGCTGTGCTCGTCATTTC
TGGACCAAAATCTGCAAACCAGTGCTCCATCAGGGGGAAGTCTGTACCAAACAACG
CAAGAAGGGTTCTCATGGGCTGGAAATTTTCCAGCGTTGCGACTGTGCGAAGGGCCT
GTCTTGCAAAGTATGGAAAGATGCCACCTACTCCTCCAAAGCCAGACTCCATGTGTG
TCAGAAAATTTGA
b. pACE463 HSA-GS-huDKK2 C2 (M172-I259 S173P)
Amino Acid Sequence including signal peptide (underlined)
(SEQ ID NO: 16)
METDTLLLWVLLLWVPGAHASRGVFRRDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLGSMPHIKGHEGDPCLRSSDCIEGFCCARHF
WTKICKPVLHQGEVCTKQRKKGSHGLEIFQRCDCAKGLSCKVWKDATYSSKARLHVC
QKI
DNA sequence including DNA encoding signal peptide (underlined)
(SEQ ID NO: 17)
ATGGAGACAGACACACTCCTGCTATGGGTACTGCTGCTCTGGGTTCCAGGTGCTCAC
GCTTCCAGGGGTGTGTTTCGTCGAGATGCACACAAGAGTGAGGTTGCTCATCGGTTT
AAAGATTTGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAACTGAATTT
GCAAAAACATGTGTTGCTGATGAGTCAGCTGAAAATTGTGACAAATCACTTCATACC
CTTTTTGGAGACAAATTATGCACAGTTGCAACTCTTCGTGAAACCTATGGTGAAATG
GCTGACTGCTGTGCAAAACAAGAACCTGAGAGAAATGAATGCTTCTTGCAACACAA
AGATGACAACCCAAACCTCCCCCGATTGGTGAGACCAGAGGTTGATGTGATGTGCA
CTGCTTTTCATGACAATGAAGAGACATTTTTGAAAAAATACTTATATGAAATTGCCA
GAAGACATCCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGCCAAAGCT
CGATGAACTTCGGGATGAAGGGAAGGCTTCGTCTGCCAAACAGAGACTCAAGTGTG
CCAGTCTCCAAAAATTTGGAGAAAGAGCTTTCAAAGCATGGGCAGTAGCTCGCCTG
AGCCAGAGATTTCCCAAAGCTGAGTTTGCAGAAGTTTCCAAGTTAGTGACAGATCTT
ACCAAAGTCCACACGGAATGCTGCCATGGAGATCTGCTTGAATGTGCTGATGACAG
GGCGGACCTTGCCAAGTATATCTGTGAAAATCAAGATTCGATCTCCAGTAAACTGAA
GGAATGCTGTGAAAAACCTCTGTTGGAAAAATCCCACTGCATTGCCGAAGTGGAAA
ATGATGAGATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGA
CATATGAAACCACTCTAGAGAAGTGCTGTGCCGCTGCAGATCCTCATGAATGCTATG
CCAAAGTGTTCGATGAATTTAAACCTCTTGTGGAAGAGCCTCAGAATTTAATCAAAC
AAAATTGTGAGCTTTTTGAGCAGCTTGGAGAGTACAAATTCCAGAATGCGCTATTAG
TTCGTTACACCAAGAAAGTACCCCAAGTGTCAACTCCAACTCTTGTAGAGGTCTCAA
GAAACCTAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTTGCATGAG
AAAACGCCAGTAAGTGACAGAGTCACCAAATGCTGCACAGAATCCTTGGTGAACAG
GCGACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAGACA
AATCAAGAAACAAACTGCACTTGTTGAGCTTGTGAAACACAAGCCCAAGGCAACAA
AAGAGCAACTGAAAGCTGTTATGGATGATTTCGCAGCTTTTGTAGAGAAGTGCTGCA
AGGCTGACGATAAGGAGACCTGCTTTGCCGAGGAGGGTAAAAAACTTGTTGCTGCA
AGTCAAGCTGCCTTAGGCTTAGGCTCTATGCCTCATATAAAAGGGCATGAAGGAGA
CCCCTGCCTACGATCATCAGACTGCATTGAAGGGTTTTGCTGTGCTCGTCATTTCTGG
ACCAAAATCTGCAAACCAGTGCTCCATCAGGGGGAAGTCTGTACCAAACAACGCAA
GAAGGGTTCTCATGGGCTGGAAATTTTCCAGCGTTGCGACTGTGCGAAGGGCCTGTC
TTGCAAAGTATGGAAAGATGCCACCTACTCCTCCAAAGCCAGACTCCATGTGTGTCA
GAAAATTTGA
c. pACE464 HSA-GS-huDKK2 C2 (H174-I259)
Amino Acid Sequence including signal peptide (underlined)
(SEQ ID NO: 18)
METDTLLLWVLLLWVPGAHASRGVFRRDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLGSHIKGHEGDPCLRSSDCIEGFCCARHFW
TKICKPVLHQGEVCTKQRKKGSHGLEIFQRCDCAKGLSCKVWKDATYSSKARLHVCQKI
DNA sequence including DNA encoding signal peptide (underlined)
(SEQ ID NO: 19)
ATGGAGACAGACACACTCCTGCTATGGGTACTGCTGCTCTGGGTTCCAGGTGCTCAC
GCTTCCAGGGGTGTGTTTCGTCGAGATGCACACAAGAGTGAGGTTGCTCATCGGTTT
AAAGATTTGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAACTGAATTT
GCAAAAACATGTGTTGCTGATGAGTCAGCTGAAAATTGTGACAAATCACTTCATACC
CTTTTTGGAGACAAATTATGCACAGTTGCAACTCTTCGTGAAACCTATGGTGAAATG
GCTGACTGCTGTGCAAAACAAGAACCTGAGAGAAATGAATGCTTCTTGCAACACAA
AGATGACAACCCAAACCTCCCCCGATTGGTGAGACCAGAGGTTGATGTGATGTGCA
CTGCTTTTCATGACAATGAAGAGACATTTTTGAAAAAATACTTATATGAAATTGCCA
GAAGACATCCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGCCAAAGCT
CGATGAACTTCGGGATGAAGGGAAGGCTTCGTCTGCCAAACAGAGACTCAAGTGTG
CCAGTCTCCAAAAATTTGGAGAAAGAGCTTTCAAAGCATGGGCAGTAGCTCGCCTG
AGCCAGAGATTTCCCAAAGCTGAGTTTGCAGAAGTTTCCAAGTTAGTGACAGATCTT
ACCAAAGTCCACACGGAATGCTGCCATGGAGATCTGCTTGAATGTGCTGATGACAG
GGCGGACCTTGCCAAGTATATCTGTGAAAATCAAGATTCGATCTCCAGTAAACTGAA
GGAATGCTGTGAAAAACCTCTGTTGGAAAAATCCCACTGCATTGCCGAAGTGGAAA
ATGATGAGATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGA
CATATGAAACCACTCTAGAGAAGTGCTGTGCCGCTGCAGATCCTCATGAATGCTATG
CCAAAGTGTTCGATGAATTTAAACCTCTTGTGGAAGAGCCTCAGAATTTAATCAAAC
AAAATTGTGAGCTTTTTGAGCAGCTTGGAGAGTACAAATTCCAGAATGCGCTATTAG
TTCGTTACACCAAGAAAGTACCCCAAGTGTCAACTCCAACTCTTGTAGAGGTCTCAA
GAAACCTAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTTGCATGAG
AAAACGCCAGTAAGTGACAGAGTCACCAAATGCTGCACAGAATCCTTGGTGAACAG
GCGACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAGACA
AATCAAGAAACAAACTGCACTTGTTGAGCTTGTGAAACACAAGCCCAAGGCAACAA
AAGAGCAACTGAAAGCTGTTATGGATGATTTCGCAGCTTTTGTAGAGAAGTGCTGCA
AGGCTGACGATAAGGAGACCTGCTTTGCCGAGGAGGGTAAAAAACTTGTTGCTGCA
AGTCAAGCTGCCTTAGGCTTAGGCTCTCATATAAAAGGGCATGAAGGAGACCCCTG
CCTACGATCATCAGACTGCATTGAAGGGTTTTGCTGTGCTCGTCATTTCTGGACCAA
AATCTGCAAACCAGTGCTCCATCAGGGGGAAGTCTGTACCAAACAACGCAAGAAGG
GTTCTCATGGGCTGGAAATTTTCCAGCGTTGCGACTGTGCGAAGGGCCTGTCTTGCA
AAGTATGGAAAGATGCCACCTACTCCTCCAAAGCCAGACTCCATGTGTGTCAGAAA
ATTTGA
d. pACE465 HSA-GS-huDKK2 C2 (K176-I259)
Amino Acid Sequence including signal peptide (underlined)
(SEQ ID NO: 20)
METDTLLLWVLLLWVPGAHASRGVFRRDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLGSKGHEGDPCLRSSDCIEGFCCARHFWTK
ICKPVLHQGEVCTKQRKKGSHGLEIFQRCDCAKGLSCKVWKDATYSSKARLHVCQKI
DNA sequence including DNA encoding signal peptide (underlined)
(SEQ ID NO: 21)
ATGGAGACAGACACACTCCTGCTATGGGTACTGCTGCTCTGGGTTCCAGGTGCTCAC
GCTTCCAGGGGTGTGTTTCGTCGAGATGCACACAAGAGTGAGGTTGCTCATCGGTTT
AAAGATTTGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAACTGAATTT
GCAAAAACATGTGTTGCTGATGAGTCAGCTGAAAATTGTGACAAATCACTTCATACC
CTTTTTGGAGACAAATTATGCACAGTTGCAACTCTTCGTGAAACCTATGGTGAAATG
GCTGACTGCTGTGCAAAACAAGAACCTGAGAGAAATGAATGCTTCTTGCAACACAA
AGATGACAACCCAAACCTCCCCCGATTGGTGAGACCAGAGGTTGATGTGATGTGCA
CTGCTTTTCATGACAATGAAGAGACATTTTTGAAAAAATACTTATATGAAATTGCCA
GAAGACATCCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGCCAAAGCT
CGATGAACTTCGGGATGAAGGGAAGGCTTCGTCTGCCAAACAGAGACTCAAGTGTG
CCAGTCTCCAAAAATTTGGAGAAAGAGCTTTCAAAGCATGGGCAGTAGCTCGCCTG
AGCCAGAGATTTCCCAAAGCTGAGTTTGCAGAAGTTTCCAAGTTAGTGACAGATCTT
ACCAAAGTCCACACGGAATGCTGCCATGGAGATCTGCTTGAATGTGCTGATGACAG
GGCGGACCTTGCCAAGTATATCTGTGAAAATCAAGATTCGATCTCCAGTAAACTGAA
GGAATGCTGTGAAAAACCTCTGTTGGAAAAATCCCACTGCATTGCCGAAGTGGAAA
ATGATGAGATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGA
CATATGAAACCACTCTAGAGAAGTGCTGTGCCGCTGCAGATCCTCATGAATGCTATG
CCAAAGTGTTCGATGAATTTAAACCTCTTGTGGAAGAGCCTCAGAATTTAATCAAAC
AAAATTGTGAGCTTTTTGAGCAGCTTGGAGAGTACAAATTCCAGAATGCGCTATTAG
TTCGTTACACCAAGAAAGTACCCCAAGTGTCAACTCCAACTCTTGTAGAGGTCTCAA
GAAACCTAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTTGCATGAG
AAAACGCCAGTAAGTGACAGAGTCACCAAATGCTGCACAGAATCCTTGGTGAACAG
GCGACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAGACA
AATCAAGAAACAAACTGCACTTGTTGAGCTTGTGAAACACAAGCCCAAGGCAACAA
AAGAGCAACTGAAAGCTGTTATGGATGATTTCGCAGCTTTTGTAGAGAAGTGCTGCA
AGGCTGACGATAAGGAGACCTGCTTTGCCGAGGAGGGTAAAAAACTTGTTGCTGCA
AGTCAAGCTGCCTTAGGCTTAGGCTCTAAAGGGCATGAAGGAGACCCCTGCCTACG
ATCATCAGACTGCATTGAAGGGTTTTGCTGTGCTCGTCATTTCTGGACCAAAATCTG
CAAACCAGTGCTCCATCAGGGGGAAGTCTGTACCAAACAACGCAAGAAGGGTTCTC
ATGGGCTGGAAATTTTCCAGCGTTGCGACTGTGCGAAGGGCCTGTCTTGCAAAGTAT
GGAAAGATGCCACCTACTCCTCCAAAGCCAGACTCCATGTGTGTCAGAAAATTTGA
e. pACE466 HSA-GS-huDKK2 C2 (H178-I259)
Amino Acid Sequence including signal peptide (underlined)
(SEQ ID NO: 22)
METDTLLLWVLLLWVPGAHASRGVFRRDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLGSHEGDPCLRSSDCIEGFCCARHFWTKICK
PVLHQGEVCTKQRKKGSHGLEIFQRCDCAKGLSCKVWKDATYSSKARLHVCQKI
DNA sequence including DNA encoding signal peptide (underlined)
(SEQ ID NO: 23)
ATGGAGACAGACACACTCCTGCTATGGGTACTGCTGCTCTGGGTTCCAGGTGCTCAC
GCTTCCAGGGGTGTGTTTCGTCGAGATGCACACAAGAGTGAGGTTGCTCATCGGTTT
AAAGATTTGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAACTGAATTT
GCAAAAACATGTGTTGCTGATGAGTCAGCTGAAAATTGTGACAAATCACTTCATACC
CTTTTTGGAGACAAATTATGCACAGTTGCAACTCTTCGTGAAACCTATGGTGAAATG
GCTGACTGCTGTGCAAAACAAGAACCTGAGAGAAATGAATGCTTCTTGCAACACAA
AGATGACAACCCAAACCTCCCCCGATTGGTGAGACCAGAGGTTGATGTGATGTGCA
CTGCTTTTCATGACAATGAAGAGACATTTTTGAAAAAATACTTATATGAAATTGCCA
GAAGACATCCTTACTTTTATGCCCCGGAACTCCTTTTCTTTGCTAAAAGGTATAAAGC
TGCTTTTACAGAATGTTGCCAAGCTGCTGATAAAGCTGCCTGCCTGTTGCCAAAGCT
CGATGAACTTCGGGATGAAGGGAAGGCTTCGTCTGCCAAACAGAGACTCAAGTGTG
CCAGTCTCCAAAAATTTGGAGAAAGAGCTTTCAAAGCATGGGCAGTAGCTCGCCTG
AGCCAGAGATTTCCCAAAGCTGAGTTTGCAGAAGTTTCCAAGTTAGTGACAGATCTT
ACCAAAGTCCACACGGAATGCTGCCATGGAGATCTGCTTGAATGTGCTGATGACAG
GGCGGACCTTGCCAAGTATATCTGTGAAAATCAAGATTCGATCTCCAGTAAACTGAA
GGAATGCTGTGAAAAACCTCTGTTGGAAAAATCCCACTGCATTGCCGAAGTGGAAA
ATGATGAGATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGA
CATATGAAACCACTCTAGAGAAGTGCTGTGCCGCTGCAGATCCTCATGAATGCTATG
CCAAAGTGTTCGATGAATTTAAACCTCTTGTGGAAGAGCCTCAGAATTTAATCAAAC
AAAATTGTGAGCTTTTTGAGCAGCTTGGAGAGTACAAATTCCAGAATGCGCTATTAG
TTCGTTACACCAAGAAAGTACCCCAAGTGTCAACTCCAACTCTTGTAGAGGTCTCAA
GAAACCTAGGAAAAGTGGGCAGCAAATGTTGTAAACATCCTGAAGCAAAAAGAATG
CCCTGTGCAGAAGACTATCTATCCGTGGTCCTGAACCAGTTATGTGTGTTGCATGAG
AAAACGCCAGTAAGTGACAGAGTCACCAAATGCTGCACAGAATCCTTGGTGAACAG
GCGACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAGACA
AATCAAGAAACAAACTGCACTTGTTGAGCTTGTGAAACACAAGCCCAAGGCAACAA
AAGAGCAACTGAAAGCTGTTATGGATGATTTCGCAGCTTTTGTAGAGAAGTGCTGCA
AGGCTGACGATAAGGAGACCTGCTTTGCCGAGGAGGGTAAAAAACTTGTTGCTGCA
AGTCAAGCTGCCTTAGGCTTAGGCTCTCATGAAGGAGACCCCTGCCTACGATCATCA
GACTGCATTGAAGGGTTTTGCTGTGCTCGTCATTTCTGGACCAAAATCTGCAAACCA
GTGCTCCATCAGGGGGAAGTCTGTACCAAACAACGCAAGAAGGGTTCTCATGGGCT
GGAAATTTTCCAGCGTTGCGACTGTGCGAAGGGCCTGTCTTGCAAAGTATGGAAAG
ATGCCACCTACTCCTCCAAAGCCAGACTCCATGTGTGTCAGAAAATTTGA
f. pACE486 HSA-GS-huDKK2 C2 (H174-I259)
Amino Acid Sequence including signal peptide (underlined)
(SEQ ID NO: 24)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKKGSHGLEIFQRCDCAKGLSCKVWKDATYSSKARLHVCQKI
DNA sequence including DNA encoding signal peptide (underlined)
(SEQ ID NO: 25)
ATGGAGACAGACACACTCCTGCTATGGGTACTGCTGCTCTGGGTTCCAGGTGCTCAC
GCTGATGCACACAAGAGTGAGGTTGCTCATCGGTTTAAAGATTTGGGAGAAGAAAA
TTTCAAAGCCTTGGTGTTGATTGCCTTTGCTCAGTATCTTCAGCAGTGTCCATTTGAA
GATCATGTAAAATTAGTGAATGAAGTAACTGAATTTGCAAAAACATGTGTTGCTGAT
GAGTCAGCTGAAAATTGTGACAAATCACTTCATACCCTTTTTGGAGACAAATTATGC
ACAGTTGCAACTCTTCGTGAAACCTATGGTGAAATGGCTGACTGCTGTGCAAAACAA
GAACCTGAGAGAAATGAATGCTTCTTGCAACACAAAGATGACAACCCAAACCTCCC
CCGATTGGTGAGACCAGAGGTTGATGTGATGTGCACTGCTTTTCATGACAATGAAGA
GACATTTTTGAAAAAATACTTATATGAAATTGCCAGAAGACATCCTTACTTTTATGC
CCCGGAACTCCTTTTCTTTGCTAAAAGGTATAAAGCTGCTTTTACAGAATGTTGCCA
AGCTGCTGATAAAGCTGCCTGCCTGTTGCCAAAGCTCGATGAACTTCGGGATGAAGG
GAAGGCTTCGTCTGCCAAACAGAGACTCAAGTGTGCCAGTCTCCAAAAATTTGGAG
AAAGAGCTTTCAAAGCATGGGCAGTAGCTCGCCTGAGCCAGAGATTTCCCAAAGCT
GAGTTTGCAGAAGTTTCCAAGTTAGTGACAGATCTTACCAAAGTCCACACGGAATGC
TGCCATGGAGATCTGCTTGAATGTGCTGATGACAGGGCGGACCTTGCCAAGTATATC
TGTGAAAATCAAGATTCGATCTCCAGTAAACTGAAGGAATGCTGTGAAAAACCTCT
GTTGGAAAAATCCCACTGCATTGCCGAAGTGGAAAATGATGAGATGCCTGCTGACTT
GCCTTCATTAGCTGCTGATTTTGTTGAAAGTAAGGATGTTTGCAAAAACTATGCTGA
GGCAAAGGATGTCTTCCTGGGCATGTTTTTGTATGAATATGCAAGAAGGCATCCTGA
TTACTCTGTCGTGCTGCTGCTGAGACTTGCCAAGACATATGAAACCACTCTAGAGAA
GTGCTGTGCCGCTGCAGATCCTCATGAATGCTATGCCAAAGTGTTCGATGAATTTAA
ACCTCTTGTGGAAGAGCCTCAGAATTTAATCAAACAAAATTGTGAGCTTTTTGAGCA
GCTTGGAGAGTACAAATTCCAGAATGCGCTATTAGTTCGTTACACCAAGAAAGTACC
CCAAGTGTCAACTCCAACTCTTGTAGAGGTCTCAAGAAACCTAGGAAAAGTGGGCA
GCAAATGTTGTAAACATCCTGAAGCAAAAAGAATGCCCTGTGCAGAAGACTATCTA
TCCGTGGTCCTGAACCAGTTATGTGTGTTGCATGAGAAAACGCCAGTAAGTGACAGA
GTCACCAAATGCTGCACAGAATCCTTGGTGAACAGGCGACCATGCTTTTCAGCTCTG
GAAGTCGATGAAACATACGTTCCCAAAGAGTTTAATGCTGAAACATTCACCTTCCAT
GCAGATATATGCACACTTTCTGAGAAGGAGAGACAAATCAAGAAACAAACTGCACT
TGTTGAGCTTGTGAAACACAAGCCCAAGGCAACAAAAGAGCAACTGAAAGCTGTTA
TGGATGATTTCGCAGCTTTTGTAGAGAAGTGCTGCAAGGCTGACGATAAGGAGACCT
GCTTTGCCGAGGAGGGTAAAAAACTTGTTGCTGCAAGTCAAGCTGCCTTAGGCTTAG
GCTCTCATATAAAAGGGCATGAAGGAGACCCCTGCCTACGATCATCAGACTGCATTG
AAGGGTTTTGCTGTGCTCGTCATTTCTGGACCAAAATCTGCAAACCAGTGCTCCATC
AGGGGGAAGTCTGTACCAAACAACGCAAGAAGGGTTCTCATGGGCTGGAAATTTTC
CAGCGTTGCGACTGTGCGAAGGGCCTGTCTTGCAAAGTATGGAAAGATGCCACCTA
CTCCTCCAAAGCCAGACTCCATGTGTGTCAGAAAATTTGA

### Example 8: Amino Acid and DNA Sequences for ACE 462 and 511

The amino acid and nucleic acid sequences of the above-noted WT DKK2C2-HSA fusions are presented below.

a. pACE462 huDKK2 C2 (M172-I259)-G2A-HSA
Amino Acid Sequence including signal peptide
(underlined)
(SEQ ID NO: 26)
METDTLLLWVLLLWVPGAHAMSHIKGHEGDPCLRSSDCIEGFCCARHFWT
KICKPVLHQGEVCTKQRKKGSHGLEIFQRCDCAKGLSCKVWKDATYSSKA
RLHVCQKIGGASRGVFRRDAHKSEVAHRFKDLGEENFKALVLIAFAQYLQ
QCPFEDHVKLVNEVTEFAKTCVADESAENCDKSLHTLFGDKLCTVATLRE
TYGEMADCCAKQEPERNECFLQHKDDNPNLPRLVRPEVDVMCTAFHDNEE
TFLKKYLYEIARRHPYFYAPELLFFAKRYKAAFTECCQAADKAACLLPKL
DELRDEGKASSAKQRLKCASLQKFGERAFKAWAVARLSQRFPKAEFAEVS
KLVTDLTKVHTECCHGDLLECADDRADLAKYICENQDSISSKLKECCEKP
LLEKSHCIAEVENDEMPADLPSLAADFVESKDVCKNYAEAKDVFLGMFLY
EYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGK
VGSKCCKHPEAKRMPCAEDYLSVVLNQLCVLHEKTPVSDRVTKCCTESLV
NRRPCFSALEVDETYVPKEFNAETFTFHADICTLSEKERQIKKQTALVEL
VKHKPKATKEQLKAVMDDFAAFVEKCCKADDKETCFAEEGKKLVAASQAA
LGL
DNA sequence of mature peptide
(SEQ ID NO: 27)
ATGTCACATATAAAAGGGCATGAAGGAGACCCCTGCCTACGATCATCAGA
CTGCATTGAAGGGTTTTGCTGTGCTCGTCATTTCTGGACCAAAATCTGCA
AACCAGTGCTCCATCAGGGGGAAGTCTGTACCAAACAACGCAAGAAGGGT
TCTCATGGGCTGGAAATTTTCCAGCGTTGCGACTGTGCGAAGGGCCTGTC
TTGCAAAGTATGGAAAGATGCCACCTACTCCTCCAAAGCCAGACTCCATG
TGTGTCAGAAAATTGGAGGTGCCAGCAGGGGTGTGTTTCGTCGAGATGCA
CACAAGAGTGAGGTTGCTCATCGGTTTAAAGATTTGGGAGAAGAAAATTT
CAAAGCCTTGGTGTTGATTGCCTTTGCTCAGTATCTTCAGCAGTGTCCAT
TTGAAGATCATGTAAAATTAGTGAATGAAGTAACTGAATTTGCAAAAACA
TGTGTTGCTGATGAGTCAGCTGAAAATTGTGACAAATCACTTCATACCCT
TTTTGGAGACAAATTATGCACAGTTGCAACTCTTCGTGAAACCTATGGTG
AAATGGCTGACTGCTGTGCAAAACAAGAACCTGAGAGAAATGAATGCTTC
TTGCAACACAAAGATGACAACCCAAACCTCCCCCGATTGGTGAGACCAGA
GGTTGATGTGATGTGCACTGCTTTTCATGACAATGAAGAGACATTTTTGA
AAAAATACTTATATGAAATTGCCAGAAGACATCCTTACTTTTATGCCCCG
GAACTCCTTTTCTTTGCTAAAAGGTATAAAGCTGCTTTTACAGAATGTTG
CCAAGCTGCTGATAAAGCTGCCTGCCTGTTGCCAAAGCTCGATGAACTTC
GGGATGAAGGGAAGGCTTCGTCTGCCAAACAGAGACTCAAGTGTGCCAGT
CTCCAAAAATTTGGAGAAAGAGCTTTCAAAGCATGGGCAGTAGCTCGCCT
GAGCCAGAGATTTCCCAAAGCTGAGTTTGCAGAAGTTTCCAAGTTAGTGA
CAGATCTTACCAAAGTCCACACGGAATGCTGCCATGGAGATCTGCTTGAA
TGTGCTGATGACAGGGCGGACCTTGCCAAGTATATCTGTGAAAATCAAGA
TTCGATCTCCAGTAAACTGAAGGAATGCTGTGAAAAACCTCTGTTGGAAA
AATCCCACTGCATTGCCGAAGTGGAAAATGATGAGATGCCTGCTGACTTG
CCTTCATTAGCTGCTGATTTTGTTGAAAGTAAGGATGTTTGCAAAAACTA
TGCTGAGGCAAAGGATGTCTTCCTGGGCATGTTTTTGTATGAATATGCAA
GAAGGCATCCTGATTACTCTGTCGTGCTGCTGCTGAGACTTGCCAAGACA
TATGAAACCACTCTAGAGAAGTGCTGTGCCGCTGCAGATCCTCATGAATG
CTATGCCAAAGTGTTCGATGAATTTAAACCTCTTGTGGAAGAGCCTCAGA
ATTTAATCAAACAAAATTGTGAGCTTTTTGAGCAGCTTGGAGAGTACAAA
TTCCAGAATGCGCTATTAGTTCGTTACACCAAGAAAGTACCCCAAGTGTC
AACTCCAACTCTTGTAGAGGTCTCAAGAAACCTAGGAAAAGTGGGCAGCA
AATGTTGTAAACATCCTGAAGCAAAAAGAATGCCCTGTGCAGAAGACTAT
CTATCCGTGGTCCTGAACCAGTTATGTGTGTTGCATGAGAAAACGCCAGT
AAGTGACAGAGTCACCAAATGCTGCACAGAATCCTTGGTGAACAGGCGAC
CATGCTTTTCAGCTCTGGAAGTCGATGAAACATACGTTCCCAAAGAGTTT
AATGCTGAAACATTCACCTTCCATGCAGATATATGCACACTTTCTGAGAA
GGAGAGACAAATCAAGAAACAAACTGCACTTGTTGAGCTTGTGAAACACA
AGCCCAAGGCAACAAAAGAGCAACTGAAAGCTGTTATGGATGATTTCGCA
GCTTTTGTAGAGAAGTGCTGCAAGGCTGACGATAAGGAGACCTGCTTTGC
CGAGGAGGGTAAAAAACTTGTTGCTGCAAGTCAAGCTGCCTTAGGCTTAT
GA
b. pACE511 huDKK2 C2-GS-HSA
Amino Acid Sequence including signal peptide
(underlined)
(SEQ ID NO: 28)
METDTLLLWVLLLWVPGAHAHIKGHEGDPCLRSSDCIEGFCCARHFWTKI
CKPVLHQGEVCTKQRKKGSHGLEIFQRCDCAKGLSCKVWKDATYSSKARL
HVCQKIGSDAHKSEVAHRFKDLGEENFKALVLIAFAQYLQQCPFEDHVKL
VNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEI
ARRHPYFYAPELLFFAKRYKAAFTECCQAADKAACLLPKLDELRDEGKAS
SAKQRLKCASLQKFGERAFKAWAVARLSQRFPKAEFAEVSKLVTDLTKVH
TECCHGDLLECADDRADLAKYICENQDSISSKLKECCEKPLLEKSHCIAE
VENDEMPADLPSLAADFVESKDVCKNYAEAKDVFLGMFLYEYARRHPDYS
VVLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHPE
AKRMPCAEDYLSVVLNQLCVLHEKTPVSDRVTKCCTESLVNRRPCFSALE
VDETYVPKEFNAETFTFHADICTLSEKERQIKKQTALVELVKHKPKATKE
QLKAVMDDFAAFVEKCCKADDKETCFAEEGKKLVAASQAALGL
DNA sequence including DNA encoding signal peptide
(underlined)
(SEQ ID NO: 29)
ATGGAGACAGACACACTCCTGCTATGGGTACTGCTGCTCTGGGTTCCAGG
TGCTCACGCTCATATAAAAGGGCATGAAGGAGACCCCTGCCTACGATCAT
CAGACTGCATTGAAGGGTTTTGCTGTGCTCGTCATTTCTGGACCAAAATC
TGCAAACCAGTGCTCCATCAGGGGGAAGTCTGTACCAAACAACGCAAGAA
GGGTTCTCATGGGCTGGAAATTTTCCAGCGTTGCGACTGTGCGAAGGGCC
TGTCTTGCAAAGTATGGAAAGATGCCACCTACTCCTCCAAAGCCAGACTC
CATGTGTGTCAGAAAATTGGATCCGATGCACACAAGAGTGAGGTTGCTCA
TCGGTTTAAAGATTTGGGAGAAGAAAATTTCAAAGCCTTGGTGTTGATTG
CCTTTGCTCAGTATCTTCAGCAGTGTCCATTTGAAGATCATGTAAAATTA
GTGAATGAAGTAACTGAATTTGCAAAAACATGTGTTGCTGATGAGTCAGC
TGAAAATTGTGACAAATCACTTCATACCCTTTTTGGAGACAAATTATGCA
CAGTTGCAACTCTTCGTGAAACCTATGGTGAAATGGCTGACTGCTGTGCA
AAACAAGAACCTGAGAGAAATGAATGCTTCTTGCAACACAAAGATGACAA
CCCAAACCTCCCCCGATTGGTGAGACCAGAGGTTGATGTGATGTGCACTG
CTTTTCATGACAATGAAGAGACATTTTTGAAAAAATACTTATATGAAATT
GCCAGAAGACATCCTTACTTTTATGCCCCGGAACTCCTTTTCTTTGCTAA
AAGGTATAAAGCTGCTTTTACAGAATGTTGCCAAGCTGCTGATAAAGCTG
CCTGCCTGTTGCCAAAGCTCGATGAACTTCGGGATGAAGGGAAGGCTTCG
TCTGCCAAACAGAGACTCAAGTGTGCCAGTCTCCAAAAATTTGGAGAAAG
AGCTTTCAAAGCATGGGCAGTAGCTCGCCTGAGCCAGAGATTTCCCAAAG
CTGAGTTTGCAGAAGTTTCCAAGTTAGTGACAGATCTTACCAAAGTCCAC
ACGGAATGCTGCCATGGAGATCTGCTTGAATGTGCTGATGACAGGGCGGA
CCTTGCCAAGTATATCTGTGAAAATCAAGATTCGATCTCCAGTAAACTGA
AGGAATGCTGTGAAAAACCTCTGTTGGAAAAATCCCACTGCATTGCCGAA
GTGGAAAATGATGAGATGCCTGCTGACTTGCCTTCATTAGCTGCTGATTT
TGTTGAAAGTAAGGATGTTTGCAAAAACTATGCTGAGGCAAAGGATGTCT
TCCTGGGCATGTTTTTGTATGAATATGCAAGAAGGCATCCTGATTACTCT
GTCGTGCTGCTGCTGAGACTTGCCAAGACATATGAAACCACTCTAGAGAA
GTGCTGTGCCGCTGCAGATCCTCATGAATGCTATGCCAAAGTGTTCGATG
AATTTAAACCTCTTGTGGAAGAGCCTCAGAATTTAATCAAACAAAATTGT
GAGCTTTTTGAGCAGCTTGGAGAGTACAAATTCCAGAATGCGCTATTAGT
TCGTTACACCAAGAAAGTACCCCAAGTGTCAACTCCAACTCTTGTAGAGG
TCTCAAGAAACCTAGGAAAAGTGGGCAGCAAATGTTGTAAACATCCTGAA
GCAAAAAGAATGCCCTGTGCAGAAGACTATCTATCCGTGGTCCTGAACCA
GTTATGTGTGTTGCATGAGAAAACGCCAGTAAGTGACAGAGTCACCAAAT
GCTGCACAGAATCCTTGGTGAACAGGCGACCATGCTTTTCAGCTCTGGAA
GTCGATGAAACATACGTTCCCAAAGAGTTTAATGCTGAAACATTCACCTT
CCATGCAGATATATGCACACTTTCTGAGAAGGAGAGACAAATCAAGAAAC
AAACTGCACTTGTTGAGCTTGTGAAACACAAGCCCAAGGCAACAAAAGAG
CAACTGAAAGCTGTTATGGATGATTTCGCAGCTTTTGTAGAGAAGTGCTG
CAAGGCTGACGATAAGGAGACCTGCTTTGCCGAGGAGGGTAAAAAACTTG
TTGCTGCAAGTCAAGCTGCCTTAGGCTTATGA

### Example 9: Design of Charge-Reversed Variants of DKK2-C2 to Reduce Heparin Binding and Drug Clearance

Heparan sulfate (HS) is a structurally varied family of sulfated glucosaminoglycans covalently attached to proteoglycans in close proximity to cell surface or extracellular matrix proteins, where HS mediates interactions between different proteins. Non-specific cell interactions through HS decrease serum exposure of proteins, resulting in reduced serum half-life. Heparin, a particular member of the HS family, is frequently used as a model compound in experimental and theoretical studies of protein-HS interactions (e.g., Mottarella et al., J. Chem. Inf. Model., 54:2068-2078 (2014)). Mutations in DKK2 C2 were created to eliminate heparin/HS binding and to decrease non-specific cell interactions through HS and thereby increase DKK2 C2 serum exposure.

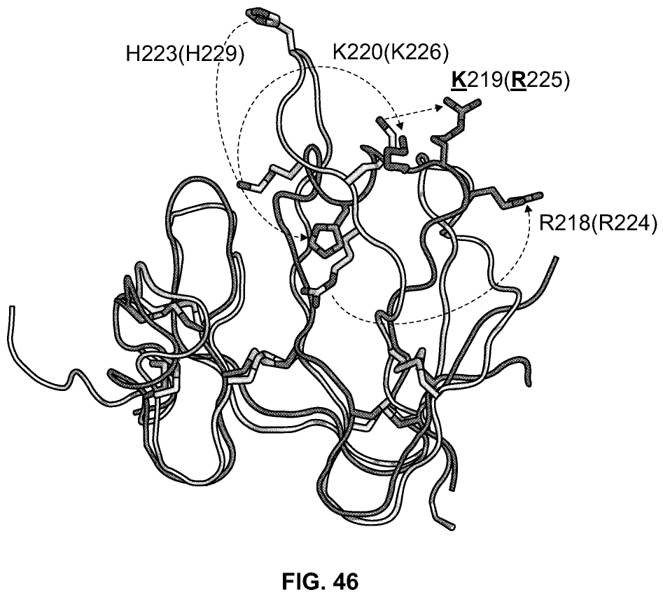

Heparin binding was reduced through charge reversal of basic residues (Lys, Arg, or His, which were changed into Glu or Asn) that constitute basic patches on the DKK2 surface distal to the binding interface between DKK2-C2 and receptor LRP6. Patch formation was estimated by structure inspection and computational analysis of the electrostatic surfaces. Crystal structures deposited in the Protein Data Bank (www.rcsb.org, Berman, J. et al., “The Protein Data Bank”; Nucleic Acids Research, 28: 235-242 (2000)) were analyzed. Considering the structures of human unbound DKK2-C2 (NMR, 2JTK.pdb) and human DKK1-C2, bound to human LRP6 (X-ray, 2 structures: 3S8V.pdb, and 3S2K.pdb), a significant conformational backbone shift between the two structures was noted (FIG. 46). This conformational shift was considered unlikely to be caused by the differences between the two DKK sequences: at 67% sequence identity, five preserved disulfide bonds, and largely preserved basic amino acid sequence patterns, it was assumed that DKK2-C2 and DKK1-C2 have similar conformational shifts under similar experimental conditions.

Rather, it was hypothesized that the different structures result either from conformational shifts upon binding, or from the different pH conditions at which data were collected: for the NMR structure (2JTK) at pH=5, and for the X-ray structures at 8.5(3S2K) and 8.8(3S8V). The conformational shift results in rearrangement of a number of basic residues, which in turn affects the location and shape of the basic patches observed in the electrostatic surfaces. Given two possible conformations that result in two different sets of charged patches, two sets of variants were designed to cover either conformation.

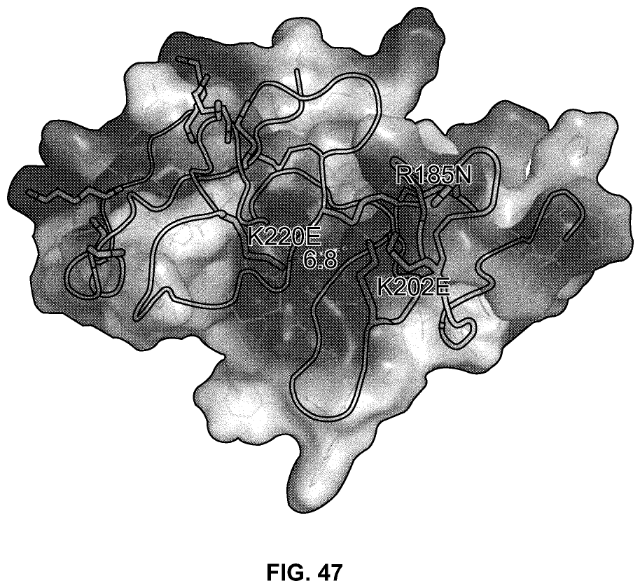

The first set of mutations was introduced based on the NMR structure of DKK2-C2 (2JTK). In this conformation, two basic patches were identified on the electrostatic surface of the protein. Patch #1 (FIG. 47) led to mutant R185N to generate a glycosylation motif 185-NSS (represented in SEQ ID NO 70, please see Table 6 for a conversion between numbering conventions between SEQ ID NO 2 and the crystal structures: in the crystals, sequence numbers are based on the full-length DKK1 and DKK2 sequences), and to the double charge reversal mutant K202E/K220E (see SEQ ID NO: 83).

TABLE 6
Sequence Number Conversion Between
SEQ ID NO: 2, and residue numbers in DKK2
(2JTK.pdb) and DKK1 (3S8V.pdb) Structures

| | Amino acid | Amino acid |
|---|---|---|
| Position # | and position | and position |
| in SEQ | in DKK2-C2 WT | in DKK1-C2 WT |
| ID NO: 2 | (2JTK.pdb) | (3S2K.pdb, 3S8V.pdb) |
| 14 | R185 | R191 |
| 26 | R197 | R203 |
| 31 | K202 | K208 |
| 45 | K216 | K222 |
| 47 | R218 | R224 |
| 48 | K219 | R225 |
| 49 | K220 | K226 |
| 69 | K240 | R246 |
| 72 | K243 | K249 |
| 79 | K250 | S257 |

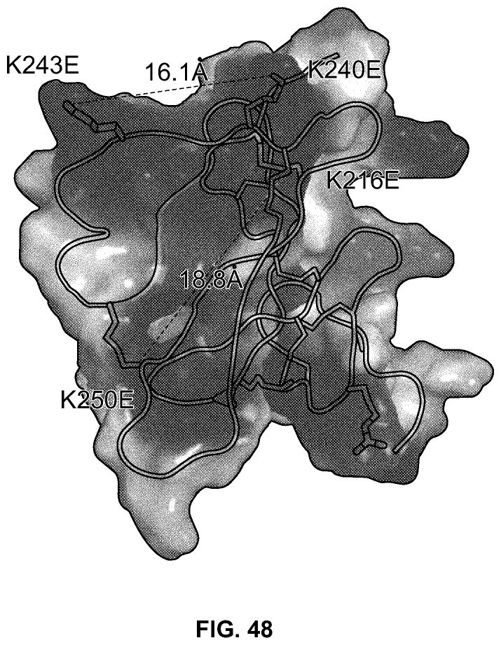

Patch #2 (FIG. 48) led to the introduction of double charge reversal mutants K240E/K243E (see SEQ ID NO: 88) and K216E/K250E (see SEQ ID NO: 85), the single charge reversal mutant K250E (SEQ ID NO 81), and the double mutant S248N/K250S (see SEQ ID NO: 90) to obtain the glycosylation motif 248-NSS.

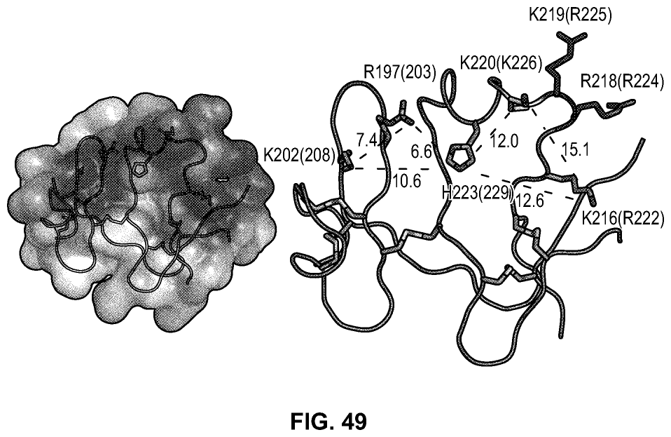

The second set of mutations was introduced on the basis of analyzing the X-ray structure of DKK1-C2, bound to LRP6 (3S8V, FIG. 49). This structure is missing coordinates for loop residues 249-KDHHQASNS, preventing observation of a basic patch in this region. On the other hand, this structure represents a conformation that is more consistent with mutational binding studies conducted to discern the correct binding interface between DKK1 and LRP6 (3S8V: Cheng Z, et al., Nat. Struct. Mol. Biol., 18: 1204-1210 (2011); 3 S2K: Ahn VE, et al., Dev. Cell, 21: 862-873 (2011)). It places residues implicated in binding into the binding site, and non-binding residues onto the distal side. In particular, residues K202, R197, H223, K220, and K216 form an extended basic patch that spans almost the entire distal side of DKK1, whereas this large patch is reduced in size in the DKK2-based structure. The placement of K220 on DKK1 is juxtaposed to K216, thus, allowing for the consideration of double mutant K216E/K220E (see, SEQ ID NO: 84). The other mutants obtained from inspection of the DKK1-C2 structure were the glycosylation mutant K220N (motif 220-NGS, see, SEQ ID NO: 76), the single charge reversal mutants K220E (see, SEQ ID NO:75), H223E (see, SEQ ID NO: 77), and R197E (see, SEQ ID NO: 71), and K202E (see, SEQ ID NO: 75), and the double charge reversal mutant K216E/H223E (see, SEQ ID NO: 86).

### Example 10: DKK2 C2 Domain Heparin Binding Mutants

Below are the amino acid sequences of examples of HSA-DKK2-C2 domain heparin binding mutants.

a. ACE 502 R185N 4D4915
Amino Acid Sequence including signal peptide (underlined)
(SEQ ID NO: 30)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLNSSDCIEGFCCARHFWTKICKPVLH
QGEVCTKQRKKGSHGLEIFQRCDCAKGLSCKVWKDATYSSKARLHVCQKI
b. ACE503 K202E/K220E
Amino Acid Sequence including signal peptide (underlined)
(SEQ ID NO: 31)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTEICKPVLH
QGEVCTKQRKEGSHGLEIFQRCDCAKGLSCKVWKDATYSSKARLHVCQKI
c. ACE 504 K240E/K243E
Amino Acid Sequence including signal peptide (underlined)
(SEQ ID NO: 32)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKKGSHGLEIFQRCDCAKGLSCEVWEDATYSSKARLHVCQKI
d. ACE505 K216E/K250E
Amino Acid Sequence including signal peptide (underlined)
(SEQ ID NO: 33)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTEQRKKGSHGLEIFQRCDCAKGLSCKVWKDATYSSEARLHVCQKI
e. ACE506 K250E
Amino Acid Sequence including signal peptide (underlined)
(SEQ ID NO: 34)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKKGSHGLEIFQRCDCAKGLSCKVWKDATYSSEARLHVCQKI
f. ACE507 S248N/K205S
Amino Acid Sequence including signal peptide (underlined)
(SEQ ID NO: 35)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKKGSHGLEIFQRCDCAKGLSCKVWKDATYNSSARLHVCQKI
g. pBKM233 HSA-DKK2 (H174-I259) K216S_K220S
Amino Acid Sequence including signal peptide (underlined)
(SEQ ID NO: 36)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTSQRKSGSHGLEIFQRCDCAKGLSCKVWKDATYSSKARLHVCQKI
h. pBKM232 HSA-DKK2 (H174-I259) K216S_H223T
Amino Acid Sequence including signal peptide (underlined)
(SEQ ID NO: 37)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTSQRKKGSTGLEIFQRCDCAKGLSCKVWKDATYSSKARLHVCQKI
i. pBKM231 HSA-DKK2 (H174-I259) K202E
Amino Acid Sequence including signal peptide (underlined)
(SEQ ID NO: 38)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTEICKPVLH
QGEVCTKQRKKGSHGLEIFQRCDCAKGLSCKVWKDATYSSKARLHVCQKI
j. pBKM230 HSA-DKK2 (H174-I259) R197E
Amino Acid Sequence including signal peptide (underlined)
(SEQ ID NO: 39)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCAEHFWTKICKPVLH
QGEVCTKQRKKGSHGLEIFQRCDCAKGLSCKVWKDATYSSKARLHVCQKI
k. pBKM229 HSA-DKK2 (H174-I259) K216E_K220E
Amino Acid Sequence including signal peptide (underlined)
(SEQ ID NO: 40)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTEQRKEGSHGLEIFQRCDCAKGLSCKVWKDATYSSKARLHVCQKI
l. pBKM228 HSA-DKK2 (H174-I259) K216E_H223E
Amino Acid Sequence including signal peptide (underlined)
(SEQ ID NO: 41)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTEQRKKGSEGLEIFQRCDCAKGLSCKVWKDATYSSKARLHVCQKI
m. pBKM227 HSA-DKK2 (H174-I259) H223E
Amino Acid Sequence including signal peptide (underlined)
(SEQ ID NO: 42)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKKGSEGLEIFQRCDCAKGLSCKVWKDATYSSKARLHVCQKI
n. pBKM226 HSA-DKK2 (H174-I259) K220E
Amino Acid Sequence including signal peptide (underlined)
(SEQ ID NO: 43)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKEGSHGLEIFQRCDCAKGLSCKVWKDATYSSKARLHVCQKI
o. pBKM225 HSA-DKK2 (H174-I259) K220N-gly
Amino Acid Sequence including signal peptide (underlined)
(SEQ ID NO: 44)
METDTLLLWVLLLWVPGAHADAHKSEVAHRFKDLGEENFKALVLIAFAQYLQQCPFED
HVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELL
FFAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMF
LYEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGLGSHIKGHEGDPCLRSSDCIEGFCCARHFWTKICKPVLH
QGEVCTKQRKNGSHGLEIFQRCDCAKGLSCKVWKDATYSSKARLHVCQKI

### Example 11: DKK2 C2 Domain Heparin Binding Mutants

Below are the nucleic acid sequences of examples of HSA-DKK2-C2 domain heparin binding mutants.

a. pBKM229
DNA sequence of mutant including the signal
sequence (which is underlined)
(SEQ ID NO: 45)
ATGGAGACAGACACACTCCTGCTATGGGTACTGCTGCTCTGGGTTCCAGG
TGCTCACGCTGATGCACACAAGAGTGAGGTTGCTCATCGGTTTAAAGATT
TGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAAC
TGAATTTGCAAAAACATGTGTTGCTGATGAGTCAGCTGAAAATTGTGACA
AATCACTTCATACCCTTTTTGGAGACAAATTATGCACAGTTGCAACTCTT
CGTGAAACCTATGGTGAAATGGCTGACTGCTGTGCAAAACAAGAACCTGA
GAGAAATGAATGCTTCTTGCAACACAAAGATGACAACCCAAACCTCCCCC
GATTGGTGAGACCAGAGGTTGATGTGATGTGCACTGCTTTTCATGACAAT
GAAGAGACATTTTTGAAAAAATACTTATATGAAATTGCCAGAAGACATCC
TTACTTTTATGCCCCGGAACTCCTTTTCTTTGCTAAAAGGTATAAAGCTG
CTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAG
ACTCAAGTGTGCCAGTCTCCAAAAATTTGGAGAAAGAGCTTTCAAAGCAT
GGGCAGTAGCTCGCCTGAGCCAGAGATTTCCCAAAGCTGAGTTTGCAGAA
GTTTCCAAGTTAGTGACAGATCTTACCAAAGTCCACACGGAATGCTGCCA
TGGAGATCTGCTTGAATGTGCTGATGACAGGGCGGACCTTGCCAAGTATA
TCTGTGAAAATCAAGATTCGATCTCCAGTAAACTGAAGGAATGCTGTGAA
AAACCTCTGTTGGAAAAATCCCACTGCATTGCCGAAGTGGAAAATGATGA
GATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTT
TTGTATGAATATGCAAGAAGGCATCCTGATTACTCTGTCGTGCTGCTGCT
GAGACTTGCCAAGACATATGAAACCACTCTAGAGAAGTGCTGTGCCGCTG
CAGATCCTCATGAATGCTATGCCAAAGTGTTCGATGAATTTAAACCTCTT
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAGA
AAGTACCCCAAGTGTCAACTCCAACTCTTGTAGAGGTCTCAAGAAACCTA
GGAAAAGTGGGCAGCAAATGTTGTAAACATCCTGAAGCAAAAAGAATGCC
CTGTGCAGAAGACTATCTATCCGTGGTCCTGAACCAGTTATGTGTGTTGC
ATGAGAAAACGCCAGTAAGTGACAGAGTCACCAAATGCTGCACAGAATCC
TTGGTGAACAGGCGACCATGCTTTTCAGCTCTGGAAGTCGATGAAACATA
CGTTCCCAAAGAGTTTAATGCTGAAACATTCACCTTCCATGCAGATATAT
GCACACTTTCTGAGAAGGAGAGACAAATCAAGAAACAAACTGCACTTGTT
GAGCTTGTGAAACACAAGCCCAAGGCAACAAAAGAGCAACTGAAAGCTGT
TATGGATGATTTCGCAGCTTTTGTAGAGAAGTGCTGCAAGGCTGACGATA
AGGAGACCTGCTTTGCCGAGGAGGGTAAAAAACTTGTTGCTGCAAGTCAA
GCTGCCTTAGGCTTAGGCTCTCATATAAAAGGGCATGAAGGAGACCCCTG
CCTACGATCATCAGACTGCATTGAAGGGTTTTGCTGTGCTCGTCATTTCT
GGACCAAAATCTGCAAACCAGTGCTCCATCAGGGGGAAGTCTGTACCGAA
CAACGCAAGGAGGGTTCTCATGGGCTGGAAATTTTCCAGCGTTGCGACTG
TGCGAAGGGCCTGTCTTGCAAAGTATGGAAAGATGCCACCTACTCCTCCA
AAGCCAGACTCCATGTGTGTCAGAAAATTTGA
b. pACE505 HSA-GS-huDKK2 C2 K216E/K250E
DATA sequence of mutant including the signal
sequence (which is underlined)
(SEQ ID NO: 46)
ATGGAGACAGACACACTCCTGCTATGGGTACTGCTGCTCTGGGTTCCAGG
TGCTCACGCTGATGCACACAAGAGTGAGGTTGCTCATCGGTTTAAAGATT
TGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAAC
TGAATTTGCAAAAACATGTGTTGCTGATGAGTCAGCTGAAAATTGTGACA
AATCACTTCATACCCTTTTTGGAGACAAATTATGCACAGTTGCAACTCTT
CGTGAAACCTATGGTGAAATGGCTGACTGCTGTGCAAAACAAGAACCTGA
GAGAAATGAATGCTTCTTGCAACACAAAGATGACAACCCAAACCTCCCCC
GATTGGTGAGACCAGAGGTTGATGTGATGTGCACTGCTTTTCATGACAAT
GAAGAGACATTTTTGAAAAAATACTTATATGAAATTGCCAGAAGACATCC
TTACTTTTATGCCCCGGAACTCCTTTTCTTTGCTAAAAGGTATAAAGCTG
CTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAG
ACTCAAGTGTGCCAGTCTCCAAAAATTTGGAGAAAGAGCTTTCAAAGCAT
GGGCAGTAGCTCGCCTGAGCCAGAGATTTCCCAAAGCTGAGTTTGCAGAA
GTTTCCAAGTTAGTGACAGATCTTACCAAAGTCCACACGGAATGCTGCCA
TGGAGATCTGCTTGAATGTGCTGATGACAGGGCGGACCTTGCCAAGTATA
TCTGTGAAAATCAAGATTCGATCTCCAGTAAACTGAAGGAATGCTGTGAA
AAACCTCTGTTGGAAAAATCCCACTGCATTGCCGAAGTGGAAAATGATGA
GATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTT
TTGTATGAATATGCAAGAAGGCATCCTGATTACTCTGTCGTGCTGCTGCT
GAGACTTGCCAAGACATATGAAACCACTCTAGAGAAGTGCTGTGCCGCTG
CAGATCCTCATGAATGCTATGCCAAAGTGTTCGATGAATTTAAACCTCTT
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAGA
AAGTACCCCAAGTGTCAACTCCAACTCTTGTAGAGGTCTCAAGAAACCTA
GGAAAAGTGGGCAGCAAATGTTGTAAACATCCTGAAGCAAAAAGAATGCC
CTGTGCAGAAGACTATCTATCCGTGGTCCTGAACCAGTTATGTGTGTTGC
ATGAGAAAACGCCAGTAAGTGACAGAGTCACCAAATGCTGCACAGAATCC
TTGGTGAACAGGCGACCATGCTTTTCAGCTCTGGAAGTCGATGAAACATA
CGTTCCCAAAGAGTTTAATGCTGAAACATTCACCTTCCATGCAGATATAT
GCACACTTTCTGAGAAGGAGAGACAAATCAAGAAACAAACTGCACTTGTT
GAGCTTGTGAAACACAAGCCCAAGGCAACAAAAGAGCAACTGAAAGCTGT
TATGGATGATTTCGCAGCTTTTGTAGAGAAGTGCTGCAAGGCTGACGATA
AGGAGACCTGCTTTGCCGAGGAGGGTAAAAAACTTGTTGCTGCAAGTCAA
GCTGCCTTAGGCTTAGGCTCTCATATAAAAGGGCATGAAGGAGACCCCTG
CCTACGATCATCAGACTGCATTGAAGGGTTTTGCTGTGCTCGTCATTTCT
GGACCAAAATCTGCAAACCAGTGCTCCATCAGGGGGAAGTCTGTACCGAA
CAACGCAAGAAGGGTTCTCATGGGCTGGAAATTTTCCAGCGTTGCGACTG
TGCGAAGGGCCTGTCTTGCAAAGTATGGAAAGATGCCACCTACTCCTCCG
AAGCCAGACTCCATGTGTGTCAGAAAATTTGA
c. pBKM228
DATA sequence of mutant including the signal
sequence (which is underlined)
(SEQ ID NO: 47)
ATGGAGACAGACACACTCCTGCTATGGGTACTGCTGCTCTGGGTTCCAGG
TGCTCACGCTGATGCACACAAGAGTGAGGTTGCTCATCGGTTTAAAGATT
TGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAAC
TGAATTTGCAAAAACATGTGTTGCTGATGAGTCAGCTGAAAATTGTGACA
AATCACTTCATACCCTTTTTGGAGACAAATTATGCACAGTTGCAACTCTT
CGTGAAACCTATGGTGAAATGGCTGACTGCTGTGCAAAACAAGAACCTGA
GAGAAATGAATGCTTCTTGCAACACAAAGATGACAACCCAAACCTCCCCC
GATTGGTGAGACCAGAGGTTGATGTGATGTGCACTGCTTTTCATGACAAT
GAAGAGACATTTTTGAAAAAATACTTATATGAAATTGCCAGAAGACATCC
TTACTTTTATGCCCCGGAACTCCTTTTCTTTGCTAAAAGGTATAAAGCTG
CTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAG
ACTCAAGTGTGCCAGTCTCCAAAAATTTGGAGAAAGAGCTTTCAAAGCAT
GGGCAGTAGCTCGCCTGAGCCAGAGATTTCCCAAAGCTGAGTTTGCAGAA
GTTTCCAAGTTAGTGACAGATCTTACCAAAGTCCACACGGAATGCTGCCA
TGGAGATCTGCTTGAATGTGCTGATGACAGGGCGGACCTTGCCAAGTATA
TCTGTGAAAATCAAGATTCGATCTCCAGTAAACTGAAGGAATGCTGTGAA
AAACCTCTGTTGGAAAAATCCCACTGCATTGCCGAAGTGGAAAATGATGA
GATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTT
TTGTATGAATATGCAAGAAGGCATCCTGATTACTCTGTCGTGCTGCTGCT
GAGACTTGCCAAGACATATGAAACCACTCTAGAGAAGTGCTGTGCCGCTG
CAGATCCTCATGAATGCTATGCCAAAGTGTTCGATGAATTTAAACCTCTT
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAGA
AAGTACCCCAAGTGTCAACTCCAACTCTTGTAGAGGTCTCAAGAAACCTA
GGAAAAGTGGGCAGCAAATGTTGTAAACATCCTGAAGCAAAAAGAATGCC
CTGTGCAGAAGACTATCTATCCGTGGTCCTGAACCAGTTATGTGTGTTGC
ATGAGAAAACGCCAGTAAGTGACAGAGTCACCAAATGCTGCACAGAATCC
TTGGTGAACAGGCGACCATGCTTTTCAGCTCTGGAAGTCGATGAAACATA
CGTTCCCAAAGAGTTTAATGCTGAAACATTCACCTTCCATGCAGATATAT
GCACACTTTCTGAGAAGGAGAGACAAATCAAGAAACAAACTGCACTTGTT
GAGCTTGTGAAACACAAGCCCAAGGCAACAAAAGAGCAACTGAAAGCTGT
TATGGATGATTTCGCAGCTTTTGTAGAGAAGTGCTGCAAGGCTGACGATA
AGGAGACCTGCTTTGCCGAGGAGGGTAAAAAACTTGTTGCTGCAAGTCAA
GCTGCCTTAGGCTTAGGCTCTCATATAAAAGGGCATGAAGGAGACCCCTG
CCTACGATCATCAGACTGCATTGAAGGGTTTTGCTGTGCTCGTCATTTCT
GGACCAAAATCTGCAAACCAGTGCTCCATCAGGGGGAAGTCTGTACCGAA
CAACGCAAGAAGGGTTCTGAAGGGCTGGAAATTTTCCAGCGTTGCGACTG
TGCGAAGGGCCTGTCTTGCAAAGTATGGAAAGATGCCACCTACTCCTCCA
AAGCCAGACTCCATGTGTGTCAGAAAATTTGA
d. pACE504 HSA-GS-huDKK2 C2 K240E/K243E
DATA sequence of mutant including the signal
sequence (which is underlined)
(SEQ ID NO: 48)
ATGGAGACAGACACACTCCTGCTATGGGTACTGCTGCTCTGGGTTCCAGG
TGCTCACGCTGATGCACACAAGAGTGAGGTTGCTCATCGGTTTAAAGATT
TGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAAC
TGAATTTGCAAAAACATGTGTTGCTGATGAGTCAGCTGAAAATTGTGACA
AATCACTTCATACCCTTTTTGGAGACAAATTATGCACAGTTGCAACTCTT
CGTGAAACCTATGGTGAAATGGCTGACTGCTGTGCAAAACAAGAACCTGA
GAGAAATGAATGCTTCTTGCAACACAAAGATGACAACCCAAACCTCCCCC
GATTGGTGAGACCAGAGGTTGATGTGATGTGCACTGCTTTTCATGACAAT
GAAGAGACATTTTTGAAAAAATACTTATATGAAATTGCCAGAAGACATCC
TTACTTTTATGCCCCGGAACTCCTTTTCTTTGCTAAAAGGTATAAAGCTG
CTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAG
ACTCAAGTGTGCCAGTCTCCAAAAATTTGGAGAAAGAGCTTTCAAAGCAT
GGGCAGTAGCTCGCCTGAGCCAGAGATTTCCCAAAGCTGAGTTTGCAGAA
GTTTCCAAGTTAGTGACAGATCTTACCAAAGTCCACACGGAATGCTGCCA
TGGAGATCTGCTTGAATGTGCTGATGACAGGGCGGACCTTGCCAAGTATA
TCTGTGAAAATCAAGATTCGATCTCCAGTAAACTGAAGGAATGCTGTGAA
AAACCTCTGTTGGAAAAATCCCACTGCATTGCCGAAGTGGAAAATGATGA
GATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTT
TTGTATGAATATGCAAGAAGGCATCCTGATTACTCTGTCGTGCTGCTGCT
GAGACTTGCCAAGACATATGAAACCACTCTAGAGAAGTGCTGTGCCGCTG
CAGATCCTCATGAATGCTATGCCAAAGTGTTCGATGAATTTAAACCTCTT
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAGA
AAGTACCCCAAGTGTCAACTCCAACTCTTGTAGAGGTCTCAAGAAACCTA
GGAAAAGTGGGCAGCAAATGTTGTAAACATCCTGAAGCAAAAAGAATGCC
CTGTGCAGAAGACTATCTATCCGTGGTCCTGAACCAGTTATGTGTGTTGC
ATGAGAAAACGCCAGTAAGTGACAGAGTCACCAAATGCTGCACAGAATCC
TTGGTGAACAGGCGACCATGCTTTTCAGCTCTGGAAGTCGATGAAACATA
CGTTCCCAAAGAGTTTAATGCTGAAACATTCACCTTCCATGCAGATATAT
GCACACTTTCTGAGAAGGAGAGACAAATCAAGAAACAAACTGCACTTGTT
GAGCTTGTGAAACACAAGCCCAAGGCAACAAAAGAGCAACTGAAAGCTGT
TATGGATGATTTCGCAGCTTTTGTAGAGAAGTGCTGCAAGGCTGACGATA
AGGAGACCTGCTTTGCCGAGGAGGGTAAAAAACTTGTTGCTGCAAGTCAA
GCTGCCTTAGGCTTAGGCTCTCATATAAAAGGGCATGAAGGAGACCCCTG
CCTACGATCATCAGACTGCATTGAAGGGTTTTGCTGTGCTCGTCATTTCT
GGACCAAAATCTGCAAACCAGTGCTCCATCAGGGGGAAGTCTGTACCAAA
CAACGCAAGAAGGGTTCTCATGGGCTGGAAATTTTCCAGCGTTGCGACTG
TGCGAAGGGCCTGTCTTGCGAAGTATGGGAAGATGCCACCTACTCCTCCA
AAGCCAGACTCCATGTGTGTCAGAAAATTTGA
e. pBKM233
DATA sequence of mutant including the signal
sequence (which is underlined)
(SEQ ID NO: 49)
ATGGAGACAGACACACTCCTGCTATGGGTACTGCTGCTCTGGGTTCCAGG
TGCTCACGCTGATGCACACAAGAGTGAGGTTGCTCATCGGTTTAAAGATT
TGGGAGAAGAAAATTTCAAAGCCTTGGTGTTGATTGCCTTTGCTCAGTAT
CTTCAGCAGTGTCCATTTGAAGATCATGTAAAATTAGTGAATGAAGTAAC
TGAATTTGCAAAAACATGTGTTGCTGATGAGTCAGCTGAAAATTGTGACA
AATCACTTCATACCCTTTTTGGAGACAAATTATGCACAGTTGCAACTCTT
CGTGAAACCTATGGTGAAATGGCTGACTGCTGTGCAAAACAAGAACCTGA
GAGAAATGAATGCTTCTTGCAACACAAAGATGACAACCCAAACCTCCCCC
GATTGGTGAGACCAGAGGTTGATGTGATGTGCACTGCTTTTCATGACAAT
GAAGAGACATTTTTGAAAAAATACTTATATGAAATTGCCAGAAGACATCC
TTACTTTTATGCCCCGGAACTCCTTTTCTTTGCTAAAAGGTATAAAGCTG
CTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAG
ACTCAAGTGTGCCAGTCTCCAAAAATTTGGAGAAAGAGCTTTCAAAGCAT
GGGCAGTAGCTCGCCTGAGCCAGAGATTTCCCAAAGCTGAGTTTGCAGAA
GTTTCCAAGTTAGTGACAGATCTTACCAAAGTCCACACGGAATGCTGCCA
TGGAGATCTGCTTGAATGTGCTGATGACAGGGCGGACCTTGCCAAGTATA
TCTGTGAAAATCAAGATTCGATCTCCAGTAAACTGAAGGAATGCTGTGAA
AAACCTCTGTTGGAAAAATCCCACTGCATTGCCGAAGTGGAAAATGATGA
GATGCCTGCTGACTTGCCTTCATTAGCTGCTGATTTTGTTGAAAGTAAGG
ATGTTTGCAAAAACTATGCTGAGGCAAAGGATGTCTTCCTGGGCATGTTT
TTGTATGAATATGCAAGAAGGCATCCTGATTACTCTGTCGTGCTGCTGCT
GAGACTTGCCAAGACATATGAAACCACTCTAGAGAAGTGCTGTGCCGCTG
CAGATCCTCATGAATGCTATGCCAAAGTGTTCGATGAATTTAAACCTCTT
GTGGAAGAGCCTCAGAATTTAATCAAACAAAATTGTGAGCTTTTTGAGCA
GCTTGGAGAGTACAAATTCCAGAATGCGCTATTAGTTCGTTACACCAAGA
AAGTACCCCAAGTGTCAACTCCAACTCTTGTAGAGGTCTCAAGAAACCTA
GGAAAAGTGGGCAGCAAATGTTGTAAACATCCTGAAGCAAAAAGAATGCC
CTGTGCAGAAGACTATCTATCCGTGGTCCTGAACCAGTTATGTGTGTTGC
ATGAGAAAACGCCAGTAAGTGACAGAGTCACCAAATGCTGCACAGAATCC
TTGGTGAACAGGCGACCATGCTTTTCAGCTCTGGAAGTCGATGAAACATA
CGTTCCCAAAGAGTTTAATGCTGAAACATTCACCTTCCATGCAGATATAT
GCACACTTTCTGAGAAGGAGAGACAAATCAAGAAACAAACTGCACTTGTT
GAGCTTGTGAAACACAAGCCCAAGGCAACAAAAGAGCAACTGAAAGCTGT
TATGGATGATTTCGCAGCTTTTGTAGAGAAGTGCTGCAAGGCTGACGATA
AGGAGACCTGCTTTGCCGAGGAGGGTAAAAAACTTGTTGCTGCAAGTCAA
GCTGCCTTAGGCTTAGGCTCTCATATAAAAGGGCATGAAGGAGACCCCTG
CCTACGATCATCAGACTGCATTGAAGGGTTTTGCTGTGCTCGTCATTTCT
GGACCAAAATCTGCAAACCAGTGCTCCATCAGGGGGAAGTCTGTACCAGC
CAACGCAAGAGCGGTTCTCATGGGCTGGAAATTTTCCAGCGTTGCGACTG
TGCGAAGGGCCTGTCTTGCAAAGTATGGAAAGATGCCACCTACTCCTCCA
AAGCCAGACTCCATGTGTGTCAGAAAATTTGA

### Example 12: Expression of HSA-huDKK2C2 Heparin Binding Mutants

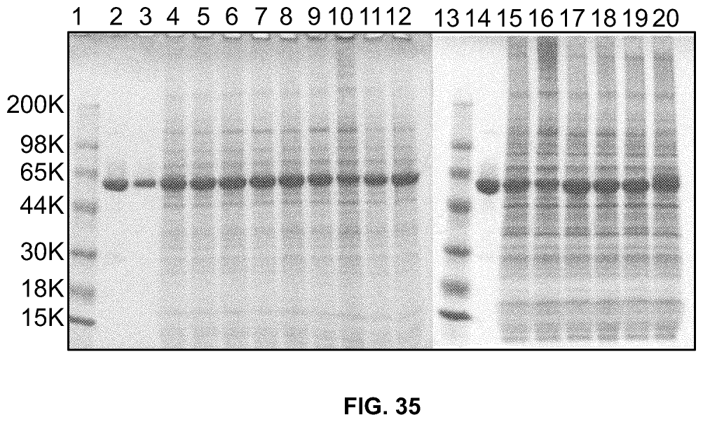

Fifteen variants of wild-type HSA-huDKK2-C2 (pACE502: HSA-huDKK2 C2 R185N; pACE503: HSA-huDKK2 C2 K202E/K220E; pACE504: HSA-huDKK2 C2 K240E/K243E; pACE505: HSA-huDKK2 C2 K216E/K250E; pACE506: HSA-huDKK2 C2 K250E; pACE507: HSA-huDKK2 C2 S248N/K205S; pBKM225: HSA-huDKK2 C2 K220N; pBKM226: HSA-huDKK2 C2 K220E; pBKM227: HSA-huDKK2 C2 H223E; pBKM228: HSA-huDKK2 C2 K216E/H223E; pBKM229: HSA-huDKK2 C2 K216E/K220E; pBKM230: HSA-huDKK2 C2 R197E; pBKM231: HSA-huDKK2 C2 K202E; pBKM232: HSA-huDKK2 C2 K216S/H223T; pBKM233: HSA-huDKK2 C2 K216S/K220S) were transiently transfected into CHO cells. For preparation of conditioned medium, cells were expanded in serum-free media, grown to high density, fed with supplements, and shifted to a reduced temperature. Cultures were held at the reduced temperature for up to 14 days or until cell viability started to drop and then harvested by centrifugation and clarified by 0.45-micron filtration. Five microliters of supernatant was examined by SDS-polyacrylamide gel electrophoresis under non-reducing conditions on a 4-20% Tris-glycine gradient gel (Invitrogen) and stained with SimplyBlue SafeStain (ThermoFisher Scientific) (FIG. 35). All of the test samples showed high level expression of HSA-DKK2 C2 as evidenced by the intensity of the prominent stained band of molecular weight of approximately 60 kDa, which migrates on SDS-PAGE at the same position as the purified wild type HSA-DKK2 C2 standard shown in lanes 2, 3 and 14. This band was not present in the conditioned medium from control cells that had not been transfected with HSA-DKK2 C2.

### Example 13: Purification of HSA-huDKK2C2 Heparin Binding Mutants

100-300 mls of transient culture was used to purify heparin-binding mutants using either SP Sepharose Fast Flow (GE Healthcare) at pH 5.0 (ACE504, BKM230, BKM231, BKM232, and BKM233), pH 5.5 (BKM225, BKM226, BKM227, ACE506 and ACE507) or pH 6.5 (ACE502, ACE506, ACE507), or using Fractogel TMAE (M) resin (Merck Millipore) at pH 7.0 (ACE503, ACE505, BKM228, and BKM229). For SP gravity purifications, 4 mls of resin was used per 300 ml of cell supernatant. Each supernatant was loaded onto a freshly poured column.

For SP-based purifications at pH 5.0, each column with resin was washed with pyrogen-free water and equilibrated with five volumes of 10 mM Citrate, 15 mM NaCl pH 5.0 (pyrogen-free) prior to the loading of cell supernatant by gravity. The columns were washed with eight column volumes of the equilibration buffer. The protein was eluted with steps containing increasing concentrations of NaCl up to 300 mM in 25 mM citrate pH 6.0, in eight fractions of 3 mls (2 fractions per concentration). Fractions were scanned for absorbance at 280 nm using a Nanodrop 2000c (ThermoFisher Scientific) and relevant fractions were pooled, filtered with a 0.2-micron filter device and dialyzed overnight into phosphate-buffered saline (PBS: 20 mM phosphate, 150 mM sodium chloride pH 7.04). Purification quality was examined by SDS-polyacrylamide gel electrophoresis and analytical size exclusion chromatography.

For SP-based purifications at pH5.5, each column with resin was washed with pyrogen-free water and equilibrated with five volumes of 10 mM Citrate, 15 mM NaCl pH5.5 (pyrogen-free) prior to the loading of supernatant by gravity. The column was washed with 2 column volumes of 15 mM citrate 50 mM NaCl pH 5.5, then 2 column volumes of PBS. The protein was eluted with 20 mM phosphate 300 mM NaCl in five fractions of 3 mls. Fractions were scanned for absorbance at 280 nm using a Nanodrop 2000c (ThermoFisher Scientific) and relevant fractions were pooled, filtered with a 0.2-micron filter device, and sodium chloride concentration adjusted to 150 mM. Purification quality was examined by SDS-polyacrylamide gel electrophoresis and analytical size exclusion chromatography.

For SP-based purifications at pH 6.5, each column with resin was washed with pyrogen-free water and equilibrated with five volumes of 10 mM Citrate, 15 mM NaCl pH 6.5 (pyrogen-free) prior to the loading of supernatant by gravity. The column was washed with 10 column volumes of equilibration buffer and protein eluted with 10 mM citrate, 1M NaCl pH6.5 in eight fractions of 2 mls. Fractions were scanned for absorbance at 280 nm using a Nanodrop 2000c (ThermoFisher Scientific) and relevant fractions were pooled, filtered with a 0.2-micron filter device, and dialyzed overnight into phosphate-buffered saline (PBS: 20 mM phosphate, 150 mM sodium chloride). Purification quality was examined by SDS-polyacrylamide gel electrophoresis and analytical size exclusion chromatography.

For TMAE gravity purifications, 10 mls of resin was used per 100 ml of supernatant. The column with resin was washed with pyrogen-free water and equilibrated with five volumes of 20 mM phosphate, 50 mM NaCl pH 7.0 (pyrogen-free) prior to the loading of supernatant by gravity. The column was washed with 2 column volumes of 20 mM phosphate, 50 mM NaCl pH 7.0. The protein was eluted with increasing concentrations of NaCl up to 450 mM in 20 mM phosphate, in ten fractions of 5 mls (2 fractions per concentration). Fractions were scanned for absorbance at 280 nm using a Nanodrop 2000c (ThermoFisher Scientific) and relevant fractions were pooled, filtered with a 0.2-micron filter device, and sodium chloride concentration adjusted to 150 mM. Purification quality was examined by SDS-polyacrylamide gel electrophoresis and analytical size exclusion chromatography.

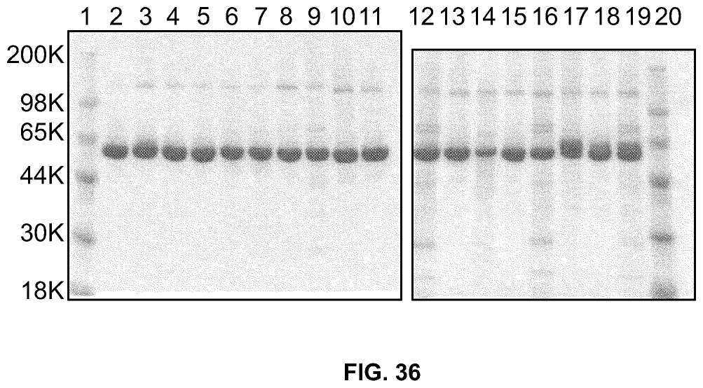

Five micrograms of all purified HSA-huDKK2 C2 mutants were examined by SDS-polyacrylamide gel electrophoresis under non-reducing conditions on a 4-20% Tris-glycine gradient gel (Invitrogen) and stained with SimplyBlue SafeStain (ThermoFisher Scientific) (FIG. 36). Average yield following ion exchange chromatography was greater than 100 mg/L. All of the purified mutants contained a single prominent HSA-DKK2 band of molecular weight of approximately 60 kDa, which migrates at the same position as the purified wild type HSA-DKK2 C2 standard shown in lane 2. The double mutate ACE507 was designed to engineer a glycosylation site into the protein. As seen in lanes 17-19, a doublet of bands at 60 kDa was observed consistent with partial glycosylation at this site. Preparations enriched in the glycosylated form (lane 17) and non-glycosylated form (lane 18) were generated during ion exchange chromatography.

For two of the constructs (BKM231 and ACE 503), there was extensive aggregation of the expressed HSA-DKK2 C2 that was evident in the SDS-PAGE analysis of conditioned medium (FIG. 35, lanes 10 and 16, respectively) as more extensive staining in the higher molecular weight region of the gel. The aggregated forms of BKM231 and ACE 503 were removed from monomer during ion exchange chromatography purification. Only the monomeric fraction was characterized in subsequent studies.

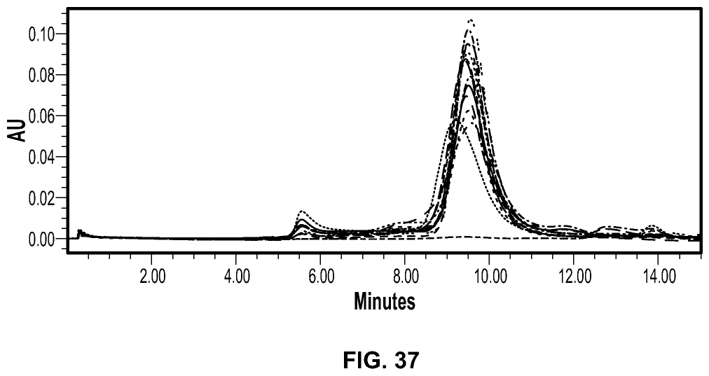

All mutants were subjected to analytical SEC on a Superdex 200 5/150 column at a flow rate of 0.2 ml/min with PBS (FIG. 37). The percent purity of all mutants was above 86% (see, Table 7 below). The SEC step revealed that all the mutants migrate as monomers, eluting from the column as a single prominent peak at 9.5 min with an apparent molecular weight of approximately 70 kDa. The broadening and shift of ACE507 S248N/K250S (pH 5.5 purification, 300 mM NaCl elution fractions 2 and 3) is consistent with glycosylation of this variant.

TABLE 7
Purity of wild-type and
mutant HSA-huDKK2 C2

| Construct | Mutations | % Purity |
|---|---|---|
| ACE464 | wild-type | 95 |
| BKM227 | H223E | 99 |
| BKM226 | K220E | 99 |
| ACE505 | K216E/K250E | 99 |
| ACE504 | K240E/K243E | 98 |
| BKM225 | K220N | 98 |
| ACE506 | K250E | 97 |
| ACE502 | R185N | 96 |
| BKM231 | K202E | 96 |
| BKM230 | R197E | 96 |
| BKM233 | K216S/K220S | 96 |
| ACE507 | S248N/K250S | 96 |
| BKM232 | K216S/H223T | 94 |
| BKM229 | K216E/K220E | 93 |
| BKM228 | K216E/H223E | 86 |

### Example 14: Mutant Characterization by Native Gel Analysis

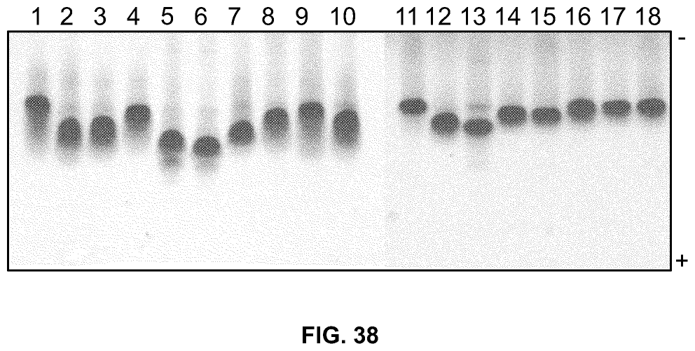

Native PAGE was used to assess the impact of changes in charge resulting from the targeted mutagenesis on the electrophoretic mobility of the HSA-DKK2 C2 constructs. Approximately 5 μg of wild-type HSA-huDKK2 C2 (ACE464) and each of the heparin binding variants was analyzed by native PAGE under non-reducing conditions on a 4-20% PAGE gradient gel (Invitrogen) and stained with SimplyBlue SafeStain (ThermoFisher Scientific) (FIG. 38). SDS was omitted from both the running buffer (50 mM acetic acid pH 5.0, adjusted with Tris base) and sample buffer (50 mM acetic acid pH 5.0, adjusted with Tris base, 25% glycerol). The gel was run at constant voltage (150V) for 4 hours. All of the samples migrated on native gels as discrete bands. The electrophoretic migration of the mutants was consistent with added negative charge resulting from the mutagenesis where the double glutamic acid mutants migrated the fastest.

### Example 15: Mutant Characterization by Heparin-Sepharose Chromatography

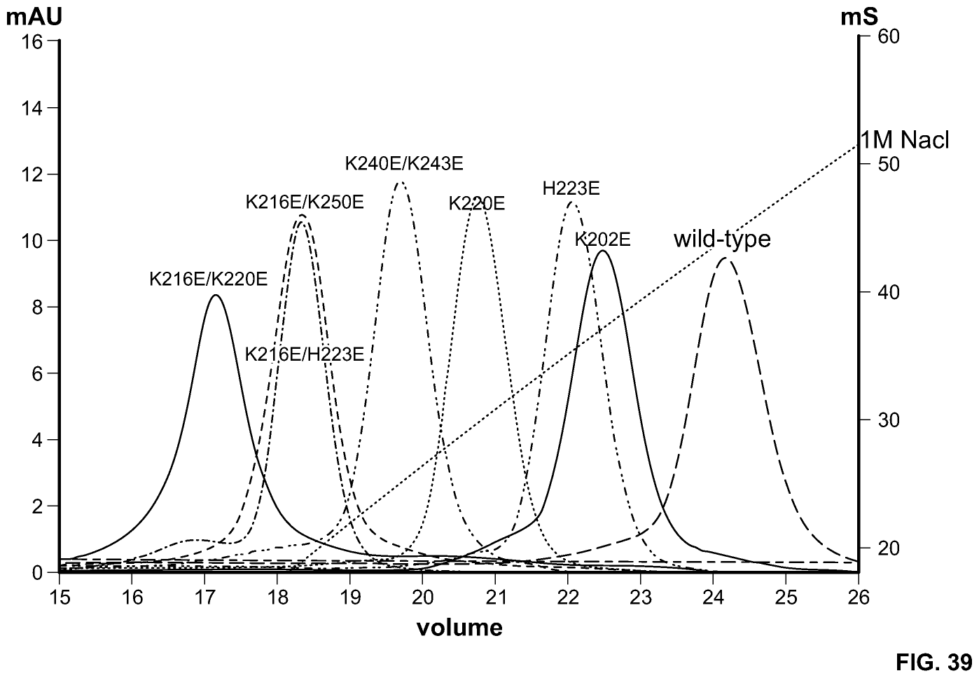

The variants were tested for their ability to bind heparin by measuring their ability to bind to a heparin-based resin and determining the salt concentration required for elution from the resin. Wild-type HSA-huDKK2 C2 (ACE464) and each of the heparin binding variants were individually subjected to heparin-sepharose chromatography under the same conditions: approximately 100 μg of material in PBS (diluted approximately 20-fold) was loaded onto a 1 ml HiTrap Heparin HP column (GE Healthcare) in binding buffer (5 mM phosphate pH 6.5). The resin was washed with 5 column volumes of binding buffer followed by elution over 20 column volumes using a linear salt gradient to 1M sodium chloride. Protein was monitored by absorbance at 280 nm and conductance in millisieverts (mS) (FIG. 39, Table 8). All mutants exhibited reduced heparin binding compared to wild type. Wildtype HSA-DKK2 C2 bound tightest to the heparin Sepharose column, where it eluted from the column with 650 mM NaCl. In contrast, the K216E/K220E mutant showed weakest binding and failed to bind the resin in the presence of 150 mM NaCl in the binding buffer. All of the mutants exhibited reduced heparin binding, eluting at lower salt with affinities that were dependent on the mutation. A detailed summary of the heparin binding results is shown in Table 8.

TABLE 8
Comparison of Elution Characteristics of Wild-type and
Mutant HSA-huDKK2 C2 from Heparin Sepharose.

| | | | Conductivity |
|---|---|---|---|
| Construct | Mutation(s) | NaCl (mM) | (mS/cm) |
| ACE503 | K202E/K220E | <150 | 13 |
| BKM229 | K216E/K220E | 300 | 16 |
| ACE505 | K216E/K250E | 370 | 22 |
| BKM228 | K216E/H223E | 370 | 22 |
| ACE504 | K240E/K243E | 440 | 29 |
| BKM233 | K216S/K220S | 450 | 31 |
| BKM226 | K220E | 490 | 33 |
| BKM225 | K220N | 490 | 34 |
| BKM232 | K216S/H223T | 490 | 34 |
| ACE506 | K250E | 540 | 38 |
| ACE507 | S248N/K250S | 560 | 40 |
| BKM227 | H223E | 560 | 40 |
| BKM230 | R197E | 560 | 40 |
| BKM231 | K202E | 580 | 42 |
| ACE502 | R185N | 590 | 42 |
| ACE464 | wild-type | 650 | 44 |

### Example 16: Mutant Characterization Examining Heparin-Biotin Binding by ELISA

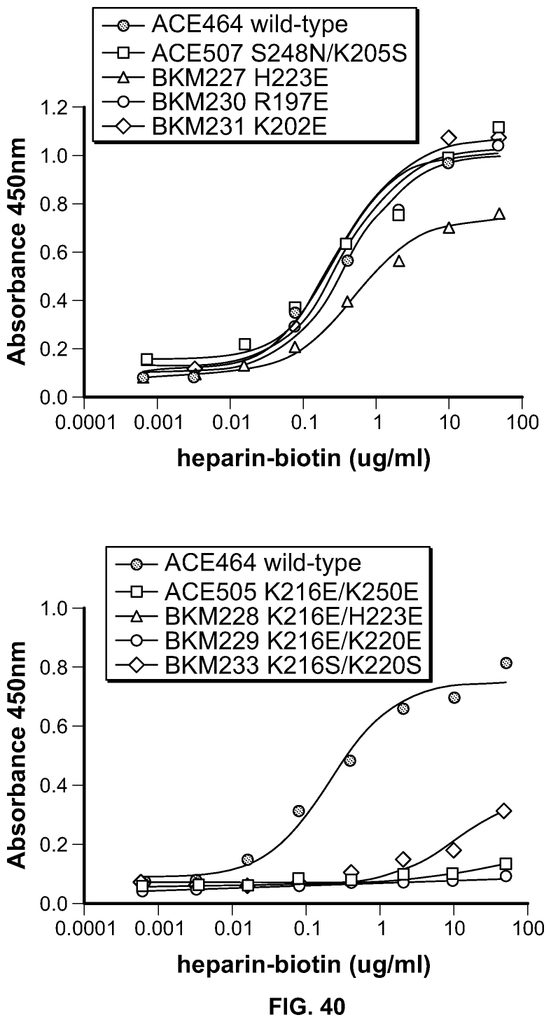

The reduced binding affinity of the mutants for heparin was confirmed using an ELISA based heparin binding assay. Wild-type HSA-huDKK2 C2 (ACE464) and each of the heparin binding variants were examined for binding to heparin-biotin using ELISA. Nunc clear flat-bottom immuno non-sterile 96-well plates (ThermoFisher Scientific) were coated with 15 μg/ml of each of the HSA-huDKK2 C2 variants and incubated overnight at 40° C. Following three washes with PBS-T (20 mM phosphate, 150 mM sodium chloride, 0.05% Tween-20), wells were incubated with ELISA blocking buffer (HBSS pH 7.0, 25 mM HEPES, 1% BSA, 0.1% ovalbumine, 0.1% NFDM, 0.001% sodium azide) at room temperature for 1 hour. Following three washes with PBS-T, wells were incubated at room temperature for 1 hour with heparin biotin sodium salt (Sigma-Aldrich) in a concentration series starting at 50 μg/ml (approximately 4 μm preceded by eight 5-fold dilutions in PBS-T, 0.05% BSA. Following three washes with PBS-T, wells were incubated at room temperature for 10 minutes with streptavidin-HRP (ThermoFisher Scientific) in a 1:8000 dilution in PBS-T, 0.05% BSA. Following two washes with PBS-T, wells were incubated at room temperature for 20 minutes with TMB substrate (0.1M NaAc citric acid pH 4.9, 0.42 mM TMB, 0.004% hydrogen peroxide). Developed ELISAs were stopped by the addition of 2N sulfuric acid and plates were scanned at 450 nm using a Molecular Devices SpectraMax M5 microplate reader (FIG. 40). IC50 values were calculated with Softmax Pro v5.4.4 software (see, Table 9 below). As observed on heparin Sepharose, the binding of the mutants to monomeric heparin was similarly impacted, where the mutants that exhibited the lowest affinity for heparin Sepharose showed the lowest affinity for monomeric heparin. From these studies five of the weakest heparin binders were selected to assess pharmacokinetics in mice. In addition to the heparin-binding mutants, two DKK variants BKM195 (H198A/K205A) and BKM199 (R230A), previously engineered to block LRP6 binding (Wang K, et al., J Biol Chem. 283:23371-5 (2008); Cheng Z, et al., Nat. Struct. Mol. Biol., 18: 1204-1210 (2011)), were tested for heparin-biotin binding. These constructs were produced as HSA-DKK2 C2 fusion proteins. Heparin binding was not impacted by these mutations.

TABLE 9
IC50 values for biotin-heparin of
HSA-huDKK2 C2 mutants binding

| | | IC50 |
|---|---|---|
| Construct | Mutation(s) | (μg/ml) |
| ACE464 | wild-type | 0.26 |
| ACE506 | K250E | 0.11 |
| ACE502 | R185N | 0.13 |
| BKM195 | H198A/K205A | 0.21 |
| BKM231 | K202E | 0.41 |
| BKM230 | R197E | 0.50 |
| BKM227 | H223E | 0.60 |
| BKM199 | R230A | 0.64 |
| ACE507 | S248N/K250S | 0.76 |
| BKM225 | K220N | >50 |
| BKM229 | K216E/K220E | >50 |
| ACE505 | K216E/K250E | >50 |
| BKM228 | K216E/H223E | >50 |
| BKM233 | K216S/K220S | >50 |
| ACE504 | K240E/K243E | >50 |
| BKM232 | K216S/H223T | >50 |
| BKM226 | K220E | >50 |
| ACE503 | K202E/K220E | >50 |

### Example 17: Mutant Characterization by Differential Scanning Fluorimetry

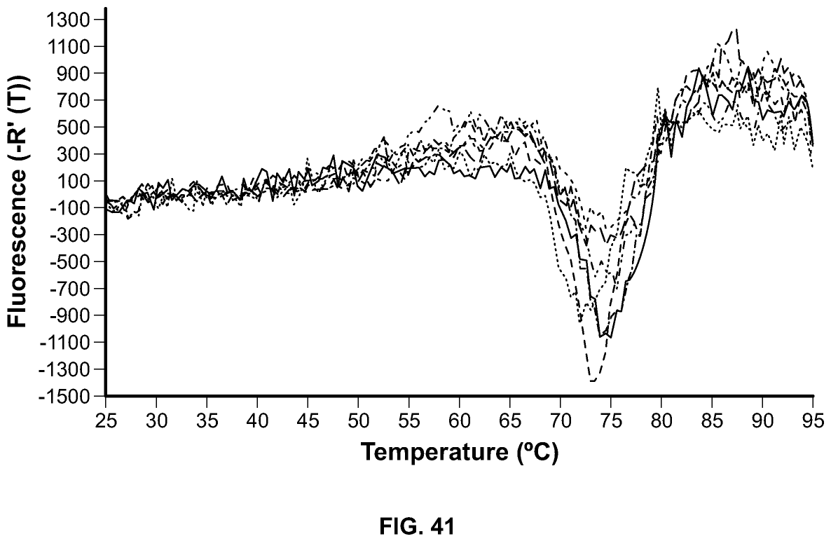

Thermal stability measurements can be used to assess product quality and solubility, where a change in the temperature at which a protein denatures is indicative of change in structure or associative forces. For these studies, approximately 100 μg of wild-type HSA-huDKK2 C2 (ACE464) and each of the heparin binding variants was diluted in 20 mM citrate-20 mM NaPi, pH 7.5, 0.1 M NaCl, to a final concentration of 2 mg/ml. SYPRO Orange (Invitrogen Molecular Probes) was added at a final 1:5000 dilution in an Applied Biosystems MicroAmp® Optical 96-Well Reaction Plate (ThermoFisher Scientific). Reactions were subjected to a method ramping temperature from 25-95° C. in 0.5° C. increments for 142 cycles on a Stratagene MX3005P (Agilent Technologies) (FIG. 41). All of the mutants were stable and similar to wildtype HSA-DKK2 C2 with observed Tm values of 72°-74° C.

### Example 18: Mutant Characterization Examining LRP6 Binding

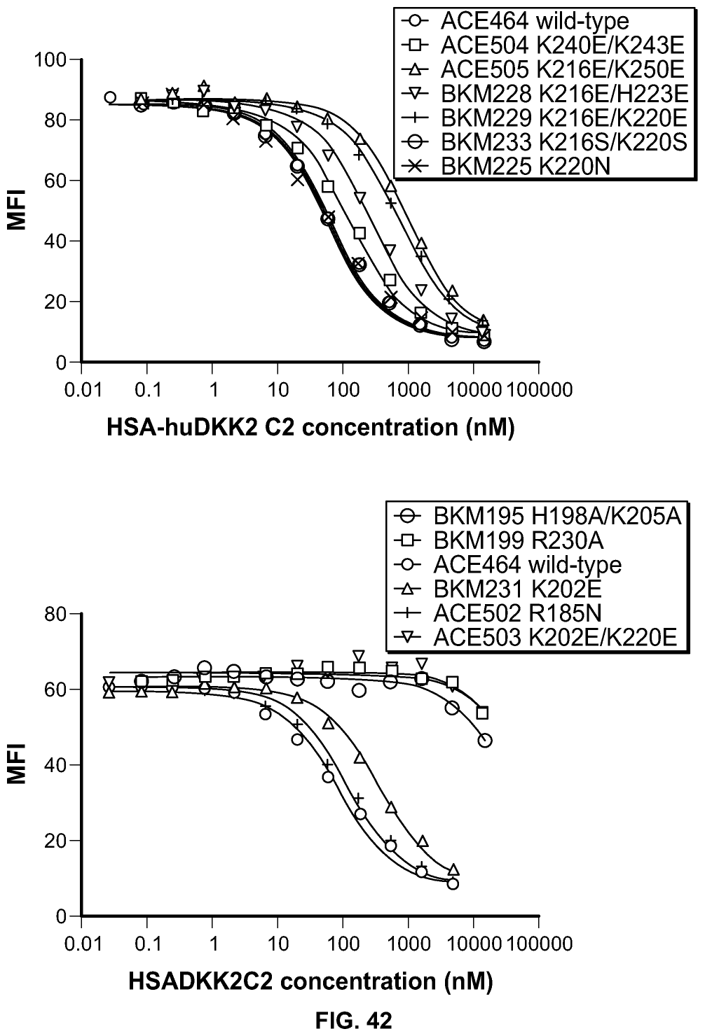

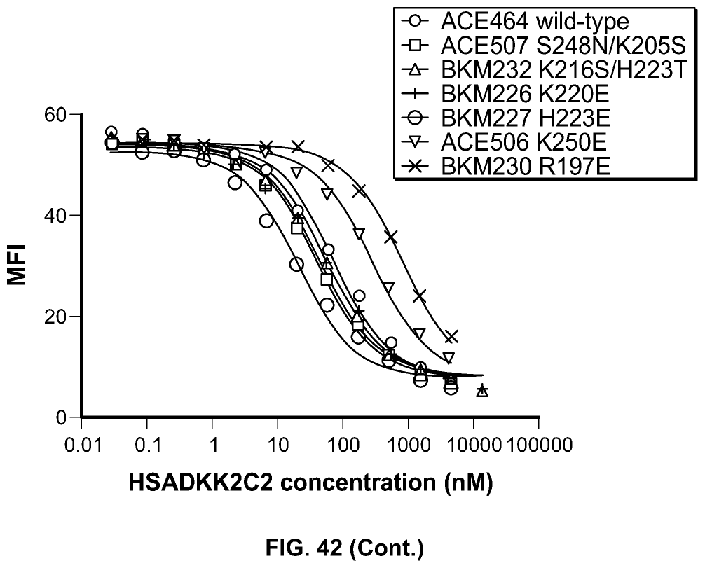

LRP6 is a cellular receptor for DKK2. To directly assess the affinity of the HSA-DKK2 C2 mutants for LRP6, we developed a FACS binding assay. Affinities were quantified by competition of binding of a high affinity antibody in a reporter format where cells were first incubated with the DKK2 variants and free LRP6 that was not bound to DKK2 was measured with the anti-LRP6 antibody. HSA-huDKK2C2 proteins were diluted at 2× concentration (final concentration ranging from 2.5-15 μM) in 100 μl cold FACS buffer (1% fetal calf serum, 20 mM phosphate, 150 mM sodium chloride, 0.05% sodium azide) in a Nunc 96-well conical bottom polypropylene plate (ThermoFisher Scientific). Eleven 3-fold serial dilutions were generated by moving 50 μl into 100 μl cold FACS buffer. huLRP6-expressing BaF3 cells (50,000/well) suspended in cold FACS buffer were distributed in 50 μl to each well and incubated at 4° C. for 1 hour. Fifty microliters of anti-LRP6 antibody (Genentech YW211.31.57 hu IgG1 agly) diluted at 4× concentration (final concentration of 0.75 nM) in cold FACS buffer was added to each well and incubated at 4° C. for 10 minutes. The plate was centrifuged at 1500 rpm for 2 minutes at 4° C. to pellet cells and supernatants were decanted. Cells were washed twice with 200 μl/well cold FACS buffer, centrifuging the plate at 1500 rpm for 2 minutes, followed by decanting. Cell pellets were re-suspended in 100 μl goat anti-human kappa-phycoerythrin (Southern Biotech) diluted 1:300 in cold FACS buffer and incubated at 4° C. for 1 hour. The plate was centrifuged at 1500 rpm for 2 minutes to pellet cells and cells were washed once with 200 μl cold FACS buffer. Cells were fixed with 150 μl/well fixation buffer (1% paraformaldehyde, 20 mM phosphate, 150 mM sodium chloride) for 10 minutes at room temperature. The plate was centrifuged at 1500 rpm for 2 minutes to pellet cells and supernatants were decanted. Cell pellets were re-suspended in 185 μl FACS buffer for analysis. In the FACS assay, wild type HSA-DKK2 C2 bound with an IC50 of 20 nM. The heparin binding mutants ranged in IC50 values of 20 nM to 1000 nM (FIG. 42). Published mutations H198A/K205A and R230A that were shown to result in loss of LRP6 binding had IC50 values of greater than 30,000 nM in this assay (Table 10).

TABLE 10
IC50 values for LRP6 binding by
HSA-huDKK2 C2 mutants

| Construct | Mutation(s) | IC50 (nM) |
|---|---|---|
| BKM227 | H223E | 21 |
| ACE507 | S248N/K250S | 42 |
| BKM226 | K220E | 51 |
| ACE486 | wild-type | 59 |
| BKM232 | K216S/H223T | 61 |
| BKM233 | K216S/K220S | 65 |
| ACE464 | wild-type | 72 |
| ACE502 | R185N | 120 |
| ACE504 | K240E/K243E | 140 |
| BKM228 | K216E/H223E | 300 |
| ACE506 | K250E | 300 |
| BKM231 | K202E | 400 |
| BKM229 | K216E/K220E | 800 |
| BKM230 | R197E | 900 |
| ACE505 | K216E/K250E | 1,000 |
| BKM195 | H198A/K205A | 35,000 |
| BKM199 | R230A | 75,000 |
| ACE503 | K202E/K220E | 100,000 |

### Example 19: Pharmacokinetics Measurements of HSA-DKK2 C2 Heparin Binding Mutants in Mice

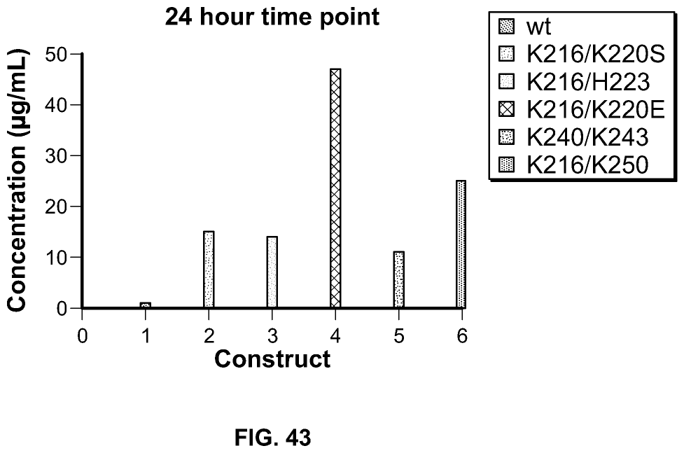

Mice (3/group) were injected intravenously with 10 mg/kg wild type and mutant forms of HSA-DKK2 C2. Blood was drawn and serum prepared after 24 hr. Levels of DKK2 in the serum were measured using a quantitative western blot protocol against a standard curve of HSA-DKK2 C2 in serum. Specifically, samples were diluted 1:10 in PBS and 7.5 ul was loaded onto a 4-12% Bis-Tris NuPAGE gel in MES buffer under non-reducing conditions. Gels were run at 200V for 35 minutes and then transferred to nitrocellulose for 7 minutes at 20V using a Life Technologies iBlot apparatus. Following 1 hour blocking in Protein-free T20 (PBS) blocking buffer (ThermoScientific Pierce) with 0.05% Tween, blots were incubated 1 hour in 1:1000 Abcam rabbit anti-DKK2 (ab95274) and 1:1000 Abcam mouse anti-HSA (ab10241) in Pierce protein-free T20 (PBS) blocking buffer with 0.2% Tween. After four washes for 5 minutes with PBSTween (0.1%), blots were incubated for 45 minutes with secondary antibodies: 1:5000 IRDye800CW donkey anti-rabbit IgG (LI-COR Biosciences) and 1:5000 IRDye680CW donkey anti-mouse (LI-COR Biosciences) in Pierce protein-free T20 (PBS) blocking buffer with 0.2% Tween. After four washes for 5 minutes with PBSTween (0.1%), blots were washed quickly four times with PBS and scanned on the Odyssey CLx reader (LI-COR Biosciences) at a PMT of 7 for both channels. Levels were quantified using Image Studio software (LI-COR Biosciences) and concentrations determined by interpolation against the standard curve. Antibodies against HSA and DKK2 C2 gave similar values. All five of the mutants tested showed much higher levels of DKK2 in the serum at the 24 hr time point (FIG. 43). For the wild type construct observed levels were less than the limit of quantitation of 1 μg/mL whereas with the mutants levels ranged from 8-47 μg/mL.

### Example 20: Assessment of Canonical Wnt Inhibition by HSA-DKK2C2 Heparin Binding Mutants

DKK2 is an inhibitor of the canonical Wnt signaling pathway. It is thought that by binding to LRP5/6, DKK2 molecules inhibit the formation of the LRP-Wnt-Frizzled ternary complex required for the activation of the canonical Wnt signaling pathway. HSA-DKK2C2 mutants were assessed for their ability to inhibit this pathway utilizing a published cell line, Super TopFlash, abbreviated as STF (Xu et al, 2004). STF is a HEK293 cell line stably transfected with a luciferase reporter under the control of 7 TCF/LEF binding sites. As the binding of TCF/LEF to its target genes is a hallmark of active canonical Wnt signaling, STF is a robust system to measure a transcriptional readout of canonical Wnt signaling.

In order to stimulate canonical Wnt signaling in this system, Wnt3a conditioned medium was generated using mouse L cells stably transfected with a full length mouse Wnt3a construct. Control conditioned medium was derived from wild type mouse L cells. All conditioned medium has a base of DMEM+10% fetal bovine serum (FBS, Hyclone). To generate conditioned medium, L cells were grown in 4 T75 flasks until 90% confluence. Medium was replenished with fresh DMEM+10% FBS and the cells incubated for an additional 48 hours. Media was collected, combined, and filter sterilized through a 0.2 μm filter. Filtered medium was then aliquoted and stored at −80° C.

STF cells were always maintained in DMEM+10% FBS. For each STF assay, STF cells were seeded into 96 well Purecoat amine plates (BD Biosciences) at a density of 4×10⁴ cells/well in a volume of 100 ul of DMEM+10% FBS. For any given experiment, 3 plates were seeded identically in order for each condition to be tested in triplicate per experiment. After 24 hours, the medium was aspirated and replaced with 100 μl of the following:

Control conditioned medium (−control)

Wnt3a conditioned medium (+control)

Wnt3a conditioned medium+HSA-DKK2C2 constructs varying from 0 nM to 1000 nM.

After 24 hours, luciferase activity was measured using the Luciferase Dual Glo kit (Promega). Dual Glo substrate was made up per the manufacturer's instructions. The medium was aspirated from the STF cells and 100 μl Dual Glo substrate was added to each well. Plates were shaken on an orbital plate shaker set to the highest setting for 2 minutes counterclockwise, and then 2 minutes clockwise. Luciferase activity was then measured on a Synergy H1 plate reader (BioTek) set to a gain of 130 with luminescence filter sets.

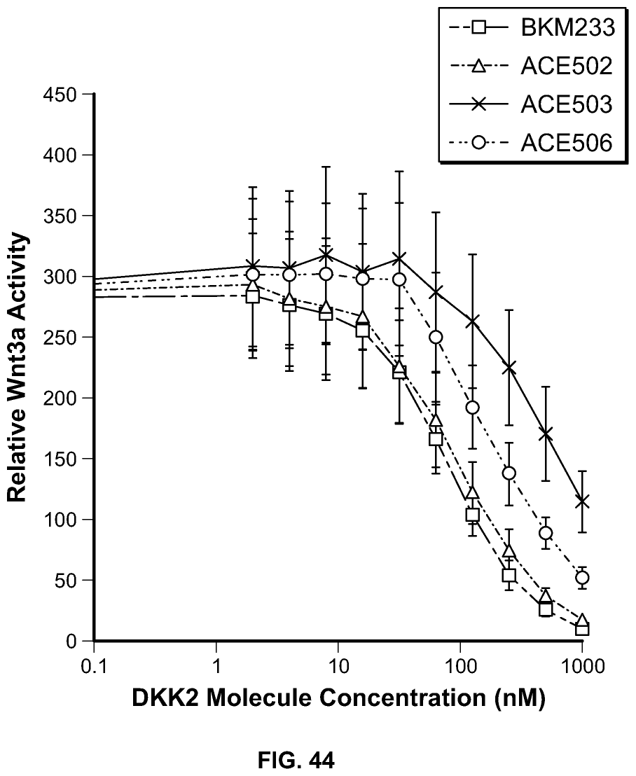

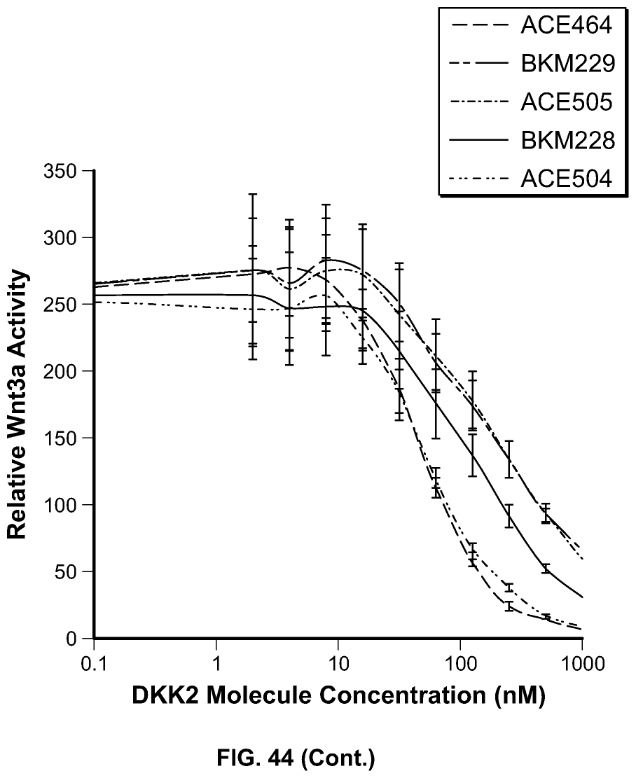

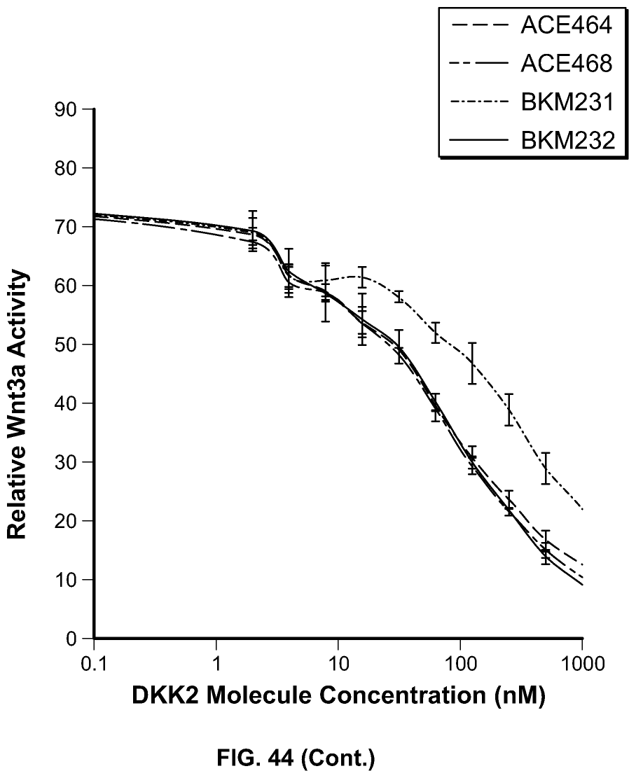

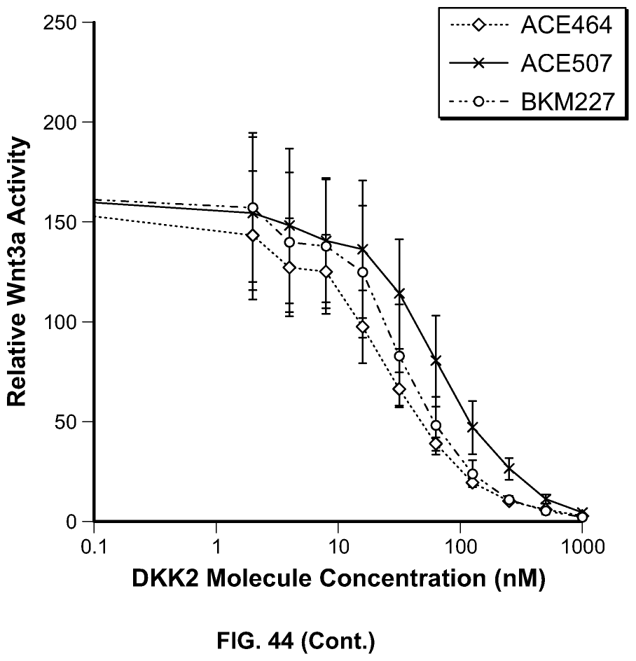

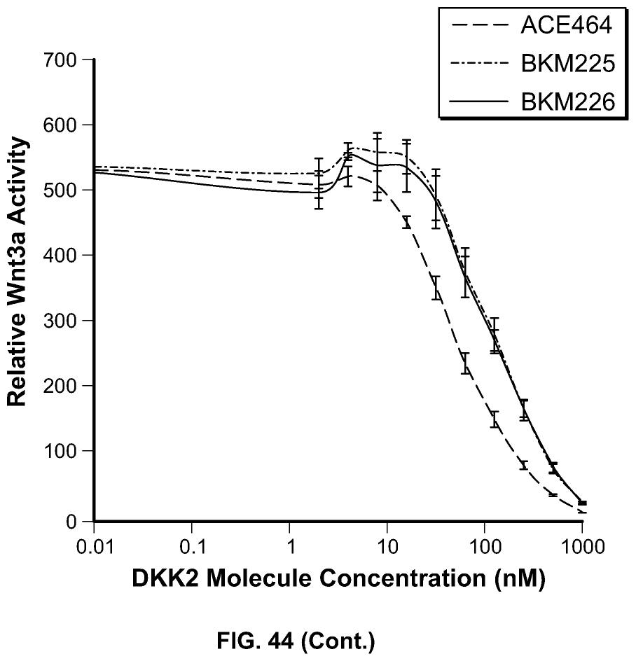

On a per plate basis, raw luminescence data was normalized to the no Wnt3a conditioned media control to derive Wnt3a induced fold changes. Plates were then averaged to generate curves of HSA-DKK2C2 induced inhibition of Wnt3a stimulated canonical Wnt signaling (FIG. 44). All of the HSA-DKK2 C2 samples tested showed dose dependent inhibition of Wnt3a stimulated signaling. All of the HSA-DKK2C2 samples tested showed dose dependent inhibition of Wnt3a stimulated canonical Wnt signaling. The constructs with wild type DKK2C2 had an apparent IC50 of 51 nM. IC50 values for the mutants are shown in Table 11.

TABLE 11
IC50 values for the Heparin-Binding Mutants

| Construct | Mutations | IC50 (nM) | St. Dev. |
|---|---|---|---|
| ACE464 | Wild Type | 51 | 22 |
| ACE486 | Wild Type | 92 | N/A |
| ACE502 | R185N | 96 | N/A |
| ACE503 | K202E/K220E | 410 | N/A |
| ACE504 | K240E/K243E | 57 | N/A |
| ACE505 | K216E/K250E | 190 | N/A |
| ACE506 | K250E | 170 | N/A |
| ACE507 | S248N/K2505 | 66 | N/A |
| BKM225 | K220N | 130 | N/A |
| BKM226 | K220E | 130 | N/A |
| BKNA227 | H223E | 34 | N/A |
| BKNA228 | K216E/H223E | 130 | N/A |
| BKNA229 | K216E/K220E | 150 | N/A |
| BKNA231 | K202E | 300 | N/A |
| BKNA232 | K216S/H223T | 110 | N/A |
| BKNA233 | K216S/K220S | 82 | N/A |

### Example 21: Assessment of Phospho-LRP6 Inhibition by HSA-DKK2C2 Heparin Binding Mutants

LRP6 phosphorylation (pLRP6) is a conserved mechanism that is required for activation of the canonical Wnt pathway. As part of its inhibitory function, DKK2 is known to prevent the phosphorylation of LRP6, thereby reducing overall pLRP6 levels.

The ability to block pLRP6 using HSA-DKK2C2 heparin binding mutants was assessed in STF cells. The same Wnt3a L cell conditioned medium and control L cell conditioned medium used in the canonical Wnt activity assay was also used in this assay. STF cells were seeded into standard 6 cm tissue culture plates at a density of 1×10⁶ cells per well in a total volume of 3 ml of DMEM+10% FBS. Once cells reached 90% confluence, the medium was aspirated and replaced with 3 ml of the following:

Control conditioned medium (−control)

Wnt3a conditioned medium (+control)

Wnt3a conditioned medium+HSA-DKK2C2 constructs varying from 0 nM to 1000 nM.

After 24 hours, cells were lysed with cold 400 μl of RIPA buffer (Millipore) containing a 1:100 dilution of HALT protease+phosphatase inhibitors (Thermo Fisher). Plates were shaken at 4° C. for 5 minutes, scraped, and the lysate transferred into pre-cooled 1.7 mL microcentrifuge tubes. An aliquot of the lysate was taken for a BCA assay (Peirce) and the remaining lysate was denatured and reduced using Bolt 10× Sample Reducing Agent (Thermo Fisher) and 4× Bolt LDS Sample Buffer (Thermo Fisher). Samples were placed at 95° C. for 5 minutes and then cooled to room temp. Reduced lysate was then passed through a QIAShredder column (Qiagen) and the resulting eluate stored at −80° C.

20 μg of total protein was loaded into each well of BOLT 4-12% Bis-Tris precast polyacrylamide gels (Thermo Fisher) and run in Bolt MES gel running buffer (Thermo Fisher). After running at 150V for 45 minutes, gels were removed, soaked in 20% ethanol on a shaker for 3 minutes, and transferred onto iBlot2 nitrocellulose (Thermo Fisher) using the iBlot2 system (Thermo Fisher) set at 20V for 13 minutes. Duplicate gels were run per sample set in order to measure pLRP6 on one gel and total LRP6 on another.

Transferred blots were blocked in a 1:1 mix of TBS and LiCor TBS blocking agent (LiCor) for 1 hr. The following antibodies were diluted 1:1000 in 1:1 mix of TBS and LiCor TBS blocking agent (LiCor):

rabbit anti-pLRP6 Ser1490 (Cell Signaling)+mouse anti-βactin (Cell Signaling, clone D6A8)

rabbit anti-LRP6 (Cell Signaling, clone C47E12)+mouse anti-βactin (Cell Signaling, clone D6A8)

All blots were incubated for 16 hours at 4° C. on a shaker.

Blots were then washed 4× in TBS+0.1% Tween20 (TBST), 5 minutes per wash on a shaker at room temperature. Blots were then incubated for 2 hours at room temperature in 1:10000 dilutions of LiCor anti-rabbit 800 (LiCor) and LiCor anti-mouse 680 (LiCor) in a 1:1 mix of TBS and LiCor TBS blocking agent (LiCor). Blots were then washed 4× in TBS+0.1% Tween20 (TB ST), 5 minutes per wash on a shaker at room temperature.

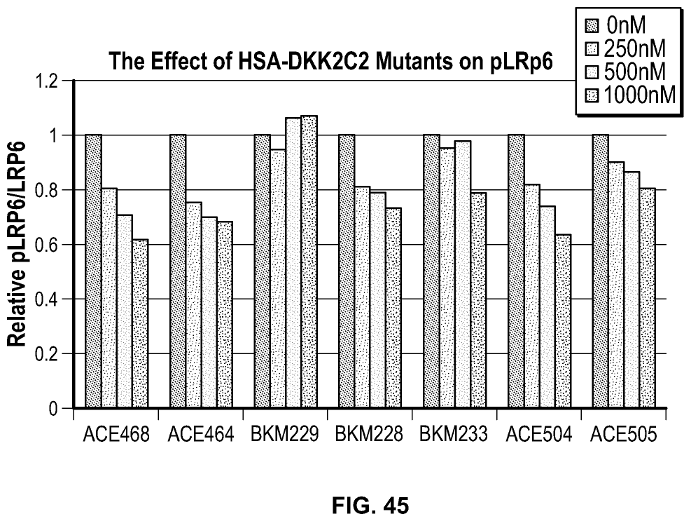

Blots were then imaged on a LiCor Odyssey CLx Imager (LiCor) and appropriate bands were then quantified using ImageStudio v 4.0 (LiCor). Per lane, pLRP6 and LRP6 values were normalized to their respective βactin values. Per condition, normalized pLRP6 values were then divided by total normalized LRP6 values to determine the proportion of pLRP6/LRP6 per condition. All values were then normalized to the pLRP6/LRP6 in control medium. Finally, all values were further normalized to the 0 nM DKK2 construct, such that for each construct series, 0 nM has a value of 1 (FIG. 45 and Table 12).

TABLE 12
%pLRP6/LRP6 at 1000 nM HSA-DKK2C2 treatment.

| | % pLRP6/LRP6 at |
|---|---|
| Construct | 1000 nM HSA-DKK2 |
| ACE468 | 62 |
| ACE464 | 68 |
| BKM229 | 110 |
| BKM228 | 73 |
| BKM233 | 79 |
| ACE504 | 64 |
| ACE505 | 81 |

The percent pLRP6/LRP6 is normalized to β-actin loading controls, no Wnt3a stimulation, and displayed as a percentage of Wnt3a alone.

HSA-DKK2C2 heparin mutants demonstrated a dose-dependent inhibition of pLRP6 that is consistent with their observed activities as canonical Wnt inhibitors in the Super TopFlash assay.

### Example 22: Mutant Characterization Examining Kremen-Biotin Binding by ELISA

To assess whether mutations designed to impact binding affinity of DKK2-C2 for heparin would impact Kremen binding, an ELISA assay monitoring Kremen-biotin binding was developed and used to measure affinities. Recombinant human Kremen-2 (R&D Systems) was dissolved to 0.2 mg/ml in PBS and incubated with EZ-Link NHS-PEG4-Biotin (ThermoScientific) to 0.08 mM final concentration at room temperature for 30 minutes. The reaction was stopped with ethanolamine and pH adjusted to pH 6.0 with 0.5 M MES pH 6.0 buffer.

Wild-type HSA-huDKK2-C2 (ACE464) and each of the heparin binding variants were examined for binding to Kremen-biotin using ELISA. Nunc clear flat-bottom immuno non-sterile 96-well plates (ThermoFisher Scientific) were coated with 30 μg/ml of each of the HSA-huDKK2-C2 variants and incubated overnight at 40° C. Following three washes with PBS-T (20 mM phosphate, 150 mM sodium chloride, 0.05% Tween-20), wells were incubated with fish gelatin blocking buffer (PBS, 0.5% fish gelatin, 0.1% Triton X-100 pH 7.4) at room temperature for 1 hour. Following three washes with PBS-T, wells were incubated at room temperature for 2 hours with Kremen-biotin in a concentration series starting at 20 μg/ml (0.4 preceded by eight 4-fold dilutions in blocking buffer. Following two washes with PBS-T, wells were incubated at room temperature for 10 minutes with streptavidin-HRP (ThermoFisher Scientific) in a 1:8000 dilution in blocking buffer. Following two washes with PBS-T, wells were incubated at room temperature for 20 minutes with TMB substrate (0.1M NaAc citric acid pH 4.9, 0.42 mM TMB, 0.004% hydrogen peroxide). Developed ELISAs were stopped by the addition of 2N sulfuric acid and plates were scanned at 450 nm using a Molecular Devices SpectraMax M5 microplate reader. Using Softmax Pro v5.4.4 software, affinities were calculated as the percentage of binding at 5 μg/ml Kremen-biotin, relative to wild type (Table 13). The binding to Kremen for the majority of mutants was affected. Mutations that included K220 had the greatest impact on Kremen binding. Mutations K250E and S248N/K250S may have potentiated Kremen binding.

TABLE 13
Relative Binding of HSA-huDKK2 C2 mutants to Kremen-Biotin
as Compared to Wild type HSA-DKK2 C2 (ACE464)
(using numbering observed in the context of
full length DKK2 for location of mutations)

| | | Percent of ACE464 |
|---|---|---|
| Construct | Mutations | binding @ 5 μg/ml |
| ACE506 | K250E | 122% |
| ACE507 | S248N/K250S | 117% |
| BKM231 | K202E | 87% |
| ACE505 | K216E/K250E | 80% |
| BKM230 | R197E | 73% |
| ACE502 | R185N | 64% |
| BKM232 | K216S/H223T | 64% |
| BKM227 | H223E | 63% |
| ACE504 | K240E/K243E | 62% |
| BKM225 | K220N | 53% |
| BKM228 | K216E/H223E | 52% |
| BKM229 | K216E/K220E | 49% |
| BKM226 | K220E | 44% |
| ACE503 | K202E/K220E | 40% |
| BKM233 | K216S/K220S | 34% |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide comprising a first amino acid sequence that is at least 90% identical to amino acids 21-605 of SEQ ID NO:24 that is directly linked or linked via a linker to a second amino acid sequence that is at least 90% identical to amino acids 3-88 of SEQ ID NO:2, wherein the polypeptide binds to LRP5/6.

2. The polypeptide of claim 1, wherein the first amino acid sequence is at least 95% identical to amino acids 21-605 of SEQ ID NO:24 and the second amino acid sequence is at least 95% identical to amino acids 3-88 of SEQ ID NO:2.

3. The polypeptide of claim 1, wherein the first amino acid sequence is identical to amino acids 21-605 of SEQ ID NO:24 and the second amino acid sequence is at least 90% identical to amino acids 3-88 of SEQ ID NO:2.

4. The polypeptide of claim 1, wherein the first amino acid sequence is identical to amino acids 21-605 of SEQ ID NO:24 and the second amino acid sequence is at least 95% identical to amino acids 3-88 of SEQ ID NO:2.

5. The polypeptide of claim 1, wherein the first amino acid sequence is identical to amino acids 21-605 of SEQ ID NO:24 and the second amino acid sequence is identical to amino acids 3-88 of SEQ ID NO:2.

6. The polypeptide of any one of claims 1 to 5, wherein the linker is a peptide linker.

7. A polypeptide comprising an amino acid sequence that is at least 90% identical to amino acids 3-88 of SEQ ID NO:2, wherein the amino acid sequence comprises at least one amino acid substitution, relative to SEQ ID NO:2, selected from the group consisting of:
(a) an amino acid other than arginine at the position corresponding to position 14 of SEQ ID NO:2;
(b) an amino acid other than arginine at the position corresponding to position 26 of SEQ ID NO:2;
(c) an amino acid other than lysine at the position corresponding to position 31 of SEQ ID NO:2;
(d) an amino acid other than lysine at the position corresponding to position 45 of SEQ ID NO:2;
(e) an amino acid other than lysine at the position corresponding to position 49 of SEQ ID NO:2;
(f) an amino acid other than histidine at the position corresponding to position 52 of SEQ ID NO:2;
(g) an amino acid other than lysine at the position corresponding to position 69 of SEQ ID NO:2;
(h) an amino acid other than lysine at the position corresponding to position 72 of SEQ ID NO:2;
(i) an amino acid other than serine at the position corresponding to position 77 of SEQ ID NO:2; and
(j) an amino acid other than lysine at the position corresponding to position 79 of SEQ ID NO:2, and
wherein the polypeptide binds to LRP5/6.

8. The polypeptide of claim 7, wherein the amino acid sequence is at least 95% identical to amino acids 3-88 of SEQ ID NO:2.

9. The polypeptide of claim 7, wherein the polypeptide comprises two amino acid substitutions selected from the group consisting of (a) through (j).

10. The polypeptide of claim 7, wherein the polypeptide comprises three amino acid substitutions selected from the group consisting of (a) through (j).

11. The polypeptide of claim 7, wherein the polypeptide comprises four amino acid substitutions selected from the group consisting of (a) through (j).

12. The polypeptide of claim 7, wherein the polypeptide contains an amino acid other than lysine at the position corresponding to position 45 of SEQ ID NO:2.

13. The polypeptide of claim 12, wherein the amino acid at the position corresponding to position 45 of SEQ ID NO:2 is glutamic acid or serine.

14. The polypeptide of any one of claims 7 to 13, wherein the polypeptide contains an amino acid other than lysine at the position corresponding to position 49 of SEQ ID NO:2.

15. The polypeptide of claim 14, wherein the amino acid at the position corresponding to position 49 of SEQ ID NO:2 is glutamic acid or asparagine.

16. The polypeptide of any one of claims 7 to 15, wherein the polypeptide contains an amino acid other than lysine at the position corresponding to position 79 of SEQ ID NO:2.

17. The polypeptide of claim 16, wherein the amino acid at the position corresponding to position 79 of SEQ ID NO:2 is glutamic acid or serine.

18. The polypeptide of any one of claims 7 to 17, wherein the polypeptide contains an amino acid other than histidine at the position corresponding to position 52 of SEQ ID NO:2.

19. The polypeptide of claim 18, wherein the amino acid at the position corresponding to position 52 of SEQ ID NO:2 is glutamic acid.

20. The polypeptide of any one of claims 7 to 19, wherein the polypeptide contains an amino acid other than lysine at the position corresponding to position 45 of SEQ ID NO:2 and an amino acid other than lysine at the position corresponding to position 49 of SEQ ID NO:2.

21. The polypeptide of claim 20, wherein the amino acids at the positions corresponding to positions 45 and 49 of SEQ ID NO:2 are glutamic acid.

22. The polypeptide of claim 20, wherein the amino acids at the positions corresponding to positions 45 and 49 of SEQ ID NO:2 are serine.

23. The polypeptide of any one of claims 7 to 22, wherein the polypeptide contains an amino acid other than lysine at the position corresponding to position 45 of SEQ ID NO:2 and an amino acid other than lysine at the position corresponding to position 79 of SEQ ID NO:2.

24. The polypeptide of claim 23, wherein the amino acids at the positions corresponding to positions 45 and 79 of SEQ ID NO:2 are glutamic acid.

25. The polypeptide of any one of claims 7 to 24, wherein the polypeptide contains an amino acid other than lysine at the position corresponding to position 45 of SEQ ID NO:2 and an amino acid other than histidine at the position corresponding to position 52 of SEQ ID NO:2.

26. The polypeptide of claim 25, wherein the amino acids at the positions corresponding to positions 45 and 52 of SEQ ID NO:2 are glutamic acid.

27. The polypeptide of claim 25, wherein the amino acid at the position corresponding to position 45 of SEQ ID NO:2 is serine and the amino acid at the position corresponding to position 52 of SEQ ID NO:2 is threonine.

28. The polypeptide of any one of claims 7 to 27, wherein the polypeptide contains an amino acid other than lysine at the position corresponding to position 69 of SEQ ID NO:2 and an amino acid other than lysine at the position corresponding to position 72 of SEQ ID NO:2.

29. The polypeptide of claim 28, wherein the amino acids at the positions corresponding to positions 69 and 72 of SEQ ID NO:2 are glutamic acid.

30. The polypeptide of any one of claims 7 to 29, wherein the polypeptide contains an amino acid other than serine at the position corresponding to position 77 of SEQ ID NO:2 and an amino acid other than lysine at the position corresponding to position 79 of SEQ ID NO:2.

31. The polypeptide of claim 30, wherein the amino acid at the position corresponding to position 77 of SEQ ID NO:2 is asparagine and the amino acid at the position corresponding to position 79 of SEQ ID NO:2 is serine.

32. The polypeptide of claim 7, wherein the amino acid sequence is identical to:
amino acids 608-693 of SEQ ID NO:32;
amino acids 608-693 of SEQ ID NO:33;
amino acids 608-693 of SEQ ID NO:36;
amino acids 608-693 of SEQ ID NO:40; or
amino acids 608-693 of SEQ ID NO:41.

33. The polypeptide of any one of claims 7 to 32, wherein the polypeptide is linked either directly or via a linker to the C-terminus of a second polypeptide comprising an amino acid sequence that is at least 90% identical to amino acids 21-605 of SEQ ID NO:24.

34. The polypeptide of any one of claims 7 to 32, wherein the polypeptide is linked either directly or via a linker to the C-terminus of a second polypeptide comprising amino acids 21-605 of SEQ ID NO:24.

35. The polypeptide of claim 34, wherein the polypeptide comprises:
amino acids 21-693 of SEQ ID NO:32;
amino acids 21-693 of SEQ ID NO:33;
amino acids 21-693 of SEQ ID NO:36;
amino acids 21-693 of SEQ ID NO:40; or
amino acids 21-693 of SEQ ID NO:41.

36. The polypeptide of any one of claims 7 to 32, wherein the polypeptide is linked either directly or via a linker to the N-terminus of a second polypeptide comprising an amino acid sequence that is at least 90% identical to amino acids 21-605 of SEQ ID NO:24.

37. The polypeptide of any one of claims 7 to 32, wherein the polypeptide is linked either directly or via a linker to the N-terminus of a second polypeptide comprising amino acids 21-605 of SEQ ID NO:24.

38. The polypeptide of any one of claim 33, 34, 36, or 37, wherein the polypeptide is linked to the second polypeptide via a linker.

39. The polypeptide of claim 38, wherein the linker is a peptide linker.

40. The polypeptide of claim 39, wherein the peptide linker is glycine-serine.

41. A pharmaceutical composition comprising the polypeptide of any one of claims 1 to 40.

42. A method of treating acute kidney injury or fibrosis, the method comprising administering to a human subject in need thereof a therapeutically effective amount of the polypeptide of any one of claims 1 to 40.

43. A nucleic acid that encodes the polypeptide of any one of claims 1 to 40.

44. A vector comprising the nucleic acid of claim 43.

45. A host cell comprising the nucleic acid of claim 43 or the vector of claim 44.

46. A method of making a polypeptide, the method comprising culturing the host cell of claim 45 under conditions that lead to the expression of the polypeptide.


SEQUENCE LISTING (102 sequences)

SEQ ID NO: 1 - protein, 107 aa, Homo sapiens
MGFLPKLLLLASFFPAGQAMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 2 - protein, 88 aa, Homo sapiens
MSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 3 - protein, 253 aa, Homo sapiens
MGFLPKLLLLASFFPAGQASQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 4 - protein, 234 aa, Homo sapiens
SQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 5 - protein, 107 aa, Mus sp.
MGFLPKLLLLASFFPAGQAMPHIKGHEGDPCLRSSDCIDGFCCARHFWTKICKPVLHQGEVCTKQRKKGSHGLEIFQRCDCAKGLSCKVWKDATYSSKARLHVCQKI

SEQ ID NO: 6 - protein, 88 aa, Mus sp.
MPHIKGHEGDPCLRSSDCIDGFCCARHFWTKICKPVLHQGEVCTKQRKKGSHGLEIFQRCDCAKGLSCKVWKDATYSSKARLHVCQKI

SEQ ID NO: 7 - protein, 253 aa, Mus sp.
MGFLPKLLLLASFFPAGQASQLGSSRAKLNSIKSSLGGETPAQSANRSAGMNQGLAFGGSKKGKSLGQAYPCSSDKECEVGRYCHSPHQGSSACMLCRRKKKRCHRDGMCCPGTRCNNGICIPVTESILTPHIPALDGTRHRDRNHGHYSNHDLGWQNLGRPHSKMPHIKGHEGDPCLRSSDCIDGFCCARHFWTKICKPVLHQGEVCTKQRKKGSHGLEIFQRCDCAKGLSCKVWKDATYSSKARLHVCQKI

SEQ ID NO: 8 - protein, 234 aa, Mus sp.
SQLGSSRAKLNSIKSSLGGETPAQSANRSAGMNQGLAFGGSKKGKSLGQAYPCSSDKECEVGRYCHSPHQGSSACMLCRRKKKRCHRDGMCCPGTRCNNGICIPVTESILTPHIPALDGTRHRDRNHGHYSNHDLGWQNLGRPHSKMPHIKGHEGDPCLRSSDCIDGFCCARHFWTKICKPVLHQGEVCTKQRKKGSHGLEIFQRCDCAKGLSCKVWKDATYSSKARLHVCQKI

SEQ ID NO: 9 - protein, 6 aa, Artificial Sequence
  source: /note=“Description of Artificial Sequence Synthetic 6xHis tag”
HHHHHH

SEQ ID NO: 10 - DNA, 771 bp, Artificial Sequence
  source: /note=“Description of Artificial Sequence Synthetic polynucleotide”
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccctggtgccgcgtggcagcatgccgcacattaaaggccatgaaggcgatccgtgcctgcgtagctctgattgcattgatggcttttgttgcgcgcgtcatttttggaccaaaatttgtaaaccggtgctgcatcagggcgaagtgtgcaccaaacagcgtaaaaaaggcagccatgggctggagatctttcagcgttgcgattgcgcgaaaggcctgagctgcaaagtgtggaaagatgcaacctatagcagcaaagcgcgtctgcatgtgtgccagaagatataatgaggatcc

SEQ ID NO: 11 - protein, 253 aa, Artificial Sequence
  source: /note=“Description of Artificial Sequence Synthetic polypeptide”
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKALVPRGSMPHIKGHEGDPCLRSSDCIDGFCCARHFWTKICKPVLHQGEVCTKQRKKGSHGLEIFQRCDCAKGLSCKVWKDATYSSKARLHVCQKI

SEQ ID NO: 12 - protein, 378 aa, Artificial Sequence
  source: /note=“Description of Artificial Sequence Synthetic polypeptide”
SPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGSQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 13 - protein, 230 aa, Artificial Sequence
  source: /note=“Description of Artificial Sequence Synthetic polypeptide”
SPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 14 - protein, 703 aa, Artificial Sequence
  source: /note=“Description of Artificial Sequence Synthetic polypeptide”
METDTLLLWVLLLWVPGAHASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAAMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 15 - DNA, 2112 bp, Artificial Sequence
  source: /note=“Description of Artificial Sequence Synthetic polynucleotide”
atggagacagacacactcctgctatgggtactgctgctctgggttccaggtgctcacgcttccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagctgccgcaatgtcacatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 16 - protein, 702 aa, Artificial Sequence
  source: /note=“Description of Artificial Sequence Synthetic polypeptide”
METDTLLLWVLLLWVPGAHASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSMPHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 17 - DNA, 2109 bp, Artificial Sequence
  source: /note=“Description of Artificial Sequence Synthetic polynucleotide”
atggagacagacacactcctgctatgggtactgctgctctgggttccaggtgctcacgcttccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctatgcctcatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 18 - protein, 700 aa, Artificial Sequence
  source: /note=“Description of Artificial Sequence Synthetic polypeptide”
METDTLLLWVLLLWVPGAHASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 19 - DNA, 2103 bp, Artificial Sequence
  source: /note=“Description of Artificial Sequence Synthetic polynucleotide”
atggagacagacacactcctgctatgggtactgctgctctgggttccaggtgctcacgcttccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 20 - protein, 698 aa, Artificial Sequence
  source: /note=“Description of Artificial Sequence Synthetic polypeptide”
METDTLLLWVLLLWVPGAHASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 21 - DNA, 2097 bp, Artificial Sequence
  source: /note=“Description of Artificial Sequence Synthetic polynucleotide”
atggagacagacacactcctgctatgggtactgctgctctgggttccaggtgctcacgcttccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 22 - protein, 696 aa, Artificial Sequence
  source: /note=“Description of Artificial Sequence Synthetic polypeptide”
METDTLLLWVLLLWVPGAHASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHEGDPCLRSSDCIEGFCCARHFWTKICKPVLHQGEVCTKQRKKGSHGLEIFQRCDCAKGLSCKVWKDATYSSKARLHVCQKI

SEQ ID NO: 23 - DNA, 2091 bp, Artificial Sequence
  source: /note=“Description of Artificial Sequence Synthetic polynucleotide”
atggagacagacacactcctgctatgggtactgctgctctgggttccaggtgctcacgcttccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 24 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 25 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 26 - protein, 703 aa, Artificial Sequence
  source: /note=“Description of Artificial Sequence Synthetic polypeptide”
METDTLLLWVLLLWVPGAHAMSHIKGHEGDPCLRSSDCIEGFCCARHFWTKICKPVLHQGEVCTKQRKKGSHGLEIFQRCDCAKGLSCKVWKDATYSSKARLHVCQKIGGASRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 27 - DNA, 2052 bp, Artificial Sequence
  source: /note=“Description of Artificial Sequence Synthetic polynucleotide”
atgtcacatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaattggaggtgccagcaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 28 - protein, 693 aa, Artificial Sequence
  source: /note=“Description of Artificial Sequence Synthetic polypeptide”
METDTLLLWVLLLWVPGAHAHIKGHEGDPCLRSSDCIEGFCCARHFWTKICKPVLHQGEVCTKQRKKGSHGLEIFQRCDCAKGLSCKVWKDATYSSKARLHVCQKIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 29 - DNA, 2082 bp, Artificial Sequence
  source: /note=“Description of Artificial Sequence Synthetic polynucleotide”
atggagacagacacactcctgctatgggtactgctgctctgggttccaggtgctcacgctcatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaattggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 30 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLNSSDCIEGFCCARHFWTKICKPVLHQGEVCTKQRKKGSHGLEIFQRCDCAKGLSCKVWKDATYSSKARLHVCQKI

SEQ ID NO: 31 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTEICKPVLHQGEVCTKQRKEGSHGLEIFQRCDCAKGLSCKVWKDATYSSKARLHVCQKI

SEQ ID NO: 32 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSHGLEIFQRCDCAKGLSCEVWEDATYSSKARLHVCQKI

SEQ ID NO: 33 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTEQRKKGSHGLEIFQRCDCAKGLSCKVWKDATYSSEARLHVCQKI

SEQ ID NO: 34 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSHGLEIFQRCDCAKGLSCKVWKDATYSSEARLHVCQKI

SEQ ID NO: 35 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSHGLEIFQRCDCAKGLSCKVWKDATYNSSARLHVCQKI

SEQ ID NO: 36 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTSQRKSGSHGLEIFQRCDCAKGLSCKVWKDATYSSKARLHVCQKI

SEQ ID NO: 37 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTSQRKKGSTGLEIFQRCDCAKGLSCKVWKDATYSSKARLHVCQKI

SEQ ID NO: 38 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTEICKPVLHQGEVCTKQRKKGSHGLEIFQRCDCAKGLSCKVWKDATYSSKARLHVCQKI

SEQ ID NO: 39 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCAEHFWTKICKPVLHQGEVCTKQRKKGSHGLEIFQRCDCAKGLSCKVWKDATYSSKARLHVCQKI

SEQ ID NO: 40 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTEQRKEGSHGLEIFQRCDCAKGLSCKVWKDATYSSKARLHVCQKI

SEQ ID NO: 41 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTEQRKKGSEGLEIFQRCDCAKGLSCKVWKDATYSSKARLHVCQKI

SEQ ID NO: 42 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKKGSEGLEIFQRCDCAKGLSCKVWKDATYSSKARLHVCQKI

SEQ ID NO: 43 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKEGSHGLEIFQRCDCAKGLSCKVWKDATYSSKARLHVCQKI

SEQ ID NO: 44 - protein, 693 aa, Artificial Sequence
  source: /note=“Description of Artificial Sequence Synthetic polypeptide”
METDTLLLWVLLLWVPGAHADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSHIKGHEGDPCLRSSDCIEGFCCARHFWTKICKPVLHQGEVCTKQRKNGSHGLEIFQRCDCAKGLSCKVWKDATYSSKARLHVCQKI

SEQ ID NO: 45 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccgaacaacgcaaggagggttctcatgggctggaaattttccagcgttgcgactgtgcgaagggcctgtcttgcaaagtatggaaagatgccacctactcctccaaagccagactccatgtgtgtcagaaaatttga

SEQ ID NO: 46 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccgaacaacgcaagaagggttctcatgggctggaaattttccagcgttgcgactgtgcgaagggcctgtcttgcaaagtatggaaagatgccacctactcctccgaagccagactccatgtgtgtcagaaaatttga

SEQ ID NO: 47 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccgaacaacgcaagaagggttctgaagggctggaaattttccagcgttgcgactgtgcgaagggcctgtcttgcaaagtatggaaagatgccacctactcctccaaagccagactccatgtgtgtcagaaaatttga

SEQ ID NO: 48 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcgaagtatgggaagatgccacctactcctccaaagccagactccatgtgtgtcagaaaatttga

SEQ ID NO: 49 - DNA, 2082 bp, Artificial Sequence
  source: /note=“Description of Artificial Sequence Synthetic polynucleotide”
atggagacagacacactcctgctatgggtactgctgctctgggttccaggtgctcacgctgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggctctcatataaaagggcatgaaggagacccctgcctacgatcatcagactgcattgaagggttttgctgtgctcgtcatttctggaccaaaatctgcaaaccagtgctccatcagggggaagtctgtaccagccaacgcaagagcggttctcatgggctggaaattttccagcgttgcgactgtgcgaagggcctgtcttgcaaagtatggaaagatgccacctactcctccaaagccagactccatgtgtgtcagaaaatttga

SEQ ID NO: 50 - protein, 585 aa, Homo sapiens [sequence omitted]

SEQ ID NO: 51 - protein, 86 aa, Homo sapiens
HIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 52 - protein, 4 aa, Artificial Sequence
  source: /note=“Description of Artificial Sequence Synthetic peptide”
GGGS

SEQ ID NO: 53 - protein, 4 aa, Artificial Sequence
  source: /note=“Description of Artificial Sequence Synthetic peptide”
SGGG

SEQ ID NO: 54 - protein, 5 aa, Artificial Sequence
  source: /note=“Description of Artificial Sequence Synthetic peptide”
GGGGS

SEQ ID NO: 55 - protein, 5 aa, Artificial Sequence
  source: /note=“Description of Artificial Sequence Synthetic peptide”
SGGGG

SEQ ID NO: 56 - protein, 6 aa, Artificial Sequence
  source: /note=“Description of Artificial Sequence Synthetic peptide”
GGGGGS

SEQ ID NO: 57 - protein, 6 aa, Artificial Sequence
  source: /note=“Description of Artificial Sequence Synthetic peptide”
SGGGGG

SEQ ID NO: 58 - protein, 7 aa, Artificial Sequence
  source: /note=“Description of Artificial Sequence Synthetic peptide”
GGGGGGS

SEQ ID NO: 59 - protein, 7 aa, Artificial Sequence
  source: /note=“Description of Artificial Sequence Synthetic peptide”
SGGGGGG

SEQ ID NO: 60 - protein, 25 aa, Artificial Sequence
  source: /note=“Description of Artificial Sequence Synthetic peptide”
  MISC_FEATURE (1)..(20): /note=“This region may encompass 0-4 ′Gly Gly Gly Xaa Xaa′ repeating units”
  MOD_RES (4)..(5): Any amino acid
  MOD_RES (9)..(10): Any amino acid
  MOD_RES (14)..(15): Any amino acid
  MOD_RES (19)..(20): Any amino acid
  source: /note=“See specification as filed for detailed description of substitutions and preferred embodiments”
GGGXXGGGXXGGGXXGGGXXGGGGS

SEQ ID NO: 61 - protein, 25 aa, Artificial Sequence
  source: /note=“Description of Artificial Sequence Synthetic peptide”
  MISC_FEATURE (6)..(25): /note=“This region may encompass 0-4 ′Xaa Gly Gly Gly Ser′ repeating units”
  MOD_RES (6)..(6): Any amino acid
  MOD_RES (11)..(11): Any amino acid
  MOD_RES (16)..(16): Any amino acid
  MOD_RES (21)..(21): Any amino acid
  source: /note=“See specification as filed for detailed description of substitutions and preferred embodiments”
GGGGSXGGGSXGGGSXGGGSXGGGS

SEQ ID NO: 62 - protein, 25 aa, Artificial Sequence
  source: /note=“Description of Artificial Sequence Synthetic peptide”
  MISC_FEATURE (1)..(20): /note=“This region may encompass 0-4 ′Gly Gly Gly Pro Ser′ repeating units”
GGGPSGGGPSGGGPSGGGPSGGGGS

SEQ ID NO: 63 - protein, 25 aa, Artificial Sequence
  source: /note=“Description of Artificial Sequence Synthetic peptide”
  MISC_FEATURE (1)..(20): /note=“This region may encompass 0-4 ′Gly Gly Gly Gly Gln′ repeating units”
GGGGQGGGGQGGGGQGGGGQGGGGS

SEQ ID NO: 64 - protein, 25 aa, Artificial Sequence
  source: /note=“Description of Artificial Sequence Synthetic peptide”
GGGGAGGGGAGGGGAGGGGAGGGGS

SEQ ID NO: 65 - protein, 25 aa, Artificial Sequence
  source: /note=“Description of Artificial Sequence Synthetic peptide”
GGGGSPGGGSPGGGSPGGGSPGGGS

SEQ ID NO: 66 - protein, 15 aa, Artificial Sequence
  source: /note=“Description of Artificial Sequence Synthetic peptide”
GGGGAGGGGAGGGGS

SEQ ID NO: 67 - protein, 15 aa, Artificial Sequence
  source: /note=“Description of Artificial Sequence Synthetic peptide”
GGGGQGGGGQGGGGS

SEQ ID NO: 68 - protein, 15 aa, Artificial Sequence
  source: /note=“Description of Artificial Sequence Synthetic peptide”
GGGPSGGGPSGGGGS

SEQ ID NO: 69 - protein, 15 aa, Artificial Sequence
  source: /note=“Description of Artificial Sequence Synthetic peptide”
GGGGSPGGGSPGGGS

SEQ ID NO: 70 - protein, 86 aa, Artificial Sequence
  source: /note=“Description of Artificial Sequence Synthetic polypeptide”
HIKGHEGDPCLNSSDCIEGFCCARHFWTKICKPVLHQGEVCTKQRKKGSHGLEIFQRCDCAKGLSCKVWKDATYSSKARLHVCQKI

SEQ ID NO: 71 - protein, 86 aa, Artificial Sequence
  source: /note=“Description of Artificial Sequence Synthetic polypeptide”
HIKGHEGDPCLRSSDCIEGFCCAEHFWTKICKPVLHQGEVCTKQRKKGSHGLEIFQRCDCAKGLSCKVWKDATYSSKARLHVCQKI

SEQ ID NO: 72 - protein, 86 aa, Artificial Sequence
  source: /note=“Description of Artificial Sequence Synthetic polypeptide”
HIKGHEGDPCLRSSDCIEGFCCARHFWTEICKPVLHQGEVCTKQRKKGSHGLEIFQRCDCAKGLSCKVWKDATYSSKARLHVCQKI

SEQ ID NO: 73 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTEQRKKGSHGLEIFQRCDCAKGLSCKVWKDATYSSKARLHVCQKI

SEQ ID NO: 74 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTSQRKKGSHGLEIFQRCDCAKGLSCKVWKDATYSSKARLHVCQKI

SEQ ID NO: 75 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKEGSHGLEIFQRCDCAKGLSCKVWKDATYSSKARLHVCQKI

SEQ ID NO: 76 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKNGSHGLEIFQRCDCAKGLSCKVWKDATYSSKARLHVCQKI

SEQ ID NO: 77 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSEGLEIFQRCDCAKGLSCKVWKDATYSSKARLHVCQKI

SEQ ID NO: 78 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCEVWKDATYSSKARLHVCQKI

SEQ ID NO: 79 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCKVWEDATYSSKARLHVCQKI

SEQ ID NO: 80 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCKVWKDATYNSKARLHVCQKI

SEQ ID NO: 81 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCKVWKDATYSSEARLHVCQKI

SEQ ID NO: 82 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCKVWKDATYSSSARLHVCQKI

SEQ ID NO: 83 - protein, 86 aa, Artificial Sequence
  source: /note=“Description of Artificial Sequence Synthetic polypeptide”
HIKGHEGDPCLRSSDCIEGFCCARHFWTEICKPVLHQGEVCTKQRKEGSHGLEIFQRCDCAKGLSCKVWKDATYSSKARLHVCQKI

SEQ ID NO: 84 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTEQRKEGSHGLEIFQRCDCAKGLSCKVWKDATYSSKARLHVCQKI

SEQ ID NO: 85 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTEQRKKGSHGLEIFQRCDCAKGLSCKVWKDATYSSEARLHVCQKI

SEQ ID NO: 86 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTEQRKKGSEGLEIFQRCDCAKGLSCKVWKDATYSSKARLHVCQKI

SEQ ID NO: 87 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTSQRKKGSTGLEIFQRCDCAKGLSCKVWKDATYSSKARLHVCQKI

SEQ ID NO: 88 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCEVWEDATYSSKARLHVCQKI

SEQ ID NO: 89 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTSQRKSGSHGLEIFQRCDCAKGLSCKVWKDATYSSKARLHVCQKI

SEQ ID NO: 90 - protein, 86 aa, Artificial Sequence
  source: /note=“Description of Artificial Sequence Synthetic polypeptide”
HIKGHEGDPCLRSSDCIEGFCCARHFWTKICKPVLHQGEVCTKQRKKGSHGLEIFQRCDCAKGLSCKVWKDATYNSSARLHVCQKI

SEQ ID NO: 91 - protein, 345 aa, Artificial Sequence
  source: /note=“Description of Artificial Sequence Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSENLYFQSMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 92 - protein, 339 aa, Artificial Sequence
  source: /note=“Description of Artificial Sequence Synthetic polypeptide”
MGFLPKLLLLASFFPAGQAMSHIKGHEGDPCLRSSDCIEGFCCARHFWTKICKPVLHQGEVCTKQRKKGSHGLEIFQRCDCAKGLSCKVWKDATYSSKARLHVCQKIENLYFQSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 5 aa, Unknown
  source: /note=“Description of Unknown mating factor alpha 1 promoter sequence”
SLDKR

SEQ ID NO: 94 - protein, 88 aa, Homo sapiens
MPHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 95 - protein, 88 aa, Xenopus sp.
IPHIKGHEGDPCLRSTDCIEGFCCARHFWTKICKPVLHQGEVCTKLRKKGSHGLEIFQRCDCAKGLSCKVWKDATYSSKSRLHICQKI

SEQ ID NO: 96 - protein, 89 aa, Mus sp.
IYHTKGQEGSVCLRSSDCAAGLCCARHFWSKICKPVLKEGQVCTKHKRKGSHGLEIFQRCYCGEGLACRIQKDHHQASNSSRLHTCQRH

SEQ ID NO: 97 - protein, 89 aa, Homo sapiens
MYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 98 - protein, 89 aa, Rattus sp.
MYHAKGQEGSVCLRSSDCATGLCCARHFWSKICKPVLKEGQVCTKHRRKGSHGLEIFQRCYCGDGLSCRLQNDQHEASNSSRLHTCQRH

SEQ ID NO: 99 - protein, 90 aa, Xenopus sp.
MQPFKGRDGDVCLRSTDCAPGLCCARHFWSKICKPVLDEGQVCTKHRRKGSHGLEIFQRCHCGAGLSCRLQKGEFTTVPKTSRLHTCQRH

SEQ ID NO: 100 - protein, 87 aa, Danio rerio
NQMLKGLEGENCLRSSDCAETLCCARHFWSKICKPVLKEGQVCTKHKRKGTHGLEIFQRCDCGEGLSCRTQRGDGGKASRSLHTCQR

SEQ ID NO: 101 - protein, 88 aa, Mus sp.
SQSSKGQEGESCLRTSDCGPGLCCARHFWTKICKPVLREGQVCSRRGHKDTAQAPEIFQRCDCGPGLTCRSQVTSNRQHSRLRVCQRI

SEQ ID NO: 102 - protein, 91 aa, Homo sapiens
SQGRKGQEGESCLRTFDCGPGLCCARHFWTKICKPVLLEGQVCSRRGHKDTAQAPEIFQRCDCGPGLLCRSQLTSNRQHARLRVCQKIEKL